(19) United States
(12) Patent Application Publication
(10) Pub. No.: US 2022/0024882 A1
(43) Pub. Date: Jan. 27, 2022

(54) NOVEL TUMOR ANTIGEN BINDING AGENTS AND USES THEREOF

(71) Applicants: ITM Isotopen Technologien München AG, Garching bei München (DE); Paul Scherrer Institut, Villigen PSI (CH)

(72) Inventors: Martina Benesova, Neckarsteinach (DE); Cristina Müller, Nussbaumen (CH); Christoph Umbricht, Baden (CH); Roger Schibli, Baden (CH); Konstantin Zhernosekov, München (DE); Luisa Maria Deberle, Zurich (CH)

(21) Appl. No.: 17/298,321

(22) PCT Filed: Nov. 28, 2019
(86) PCT No.: PCT/EP2019/083005

§ 371 Date: May 28, 2021

(30) Foreign Application Priority Data
Nov. 28, 2018 (EP) PCT/EP2018/082910

(51) Int. Cl. C07D 257/02; A61K 51/04; A61P 35/00; G01N 33/60

(52) U.S. Cl. CPC C07D 257/02; A61K 51/0402; G01N 33/60; A61P 35/00; A61K 51/0497

(57) ABSTRACT

The present invention provides compounds according to General Formula (1)(i) or (1)(ii): wherein A is a diagnostic or therapeutic agent comprising a binding site for a tumor antigen, and the spacer comprises at least one C—N bond.

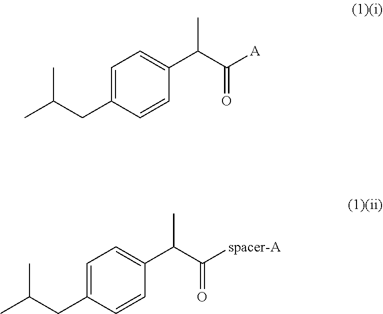

The present invention relates to novel compounds and radiolabeled complexes comprising a tumor-antigen binding site, in particular a PSMA-binding entity, and an albumin-binding entity connected via suitable linkers and spacers, which are envisaged for use as diagnostic and/or therapeutic radiopharmaceuticals. Specifically, the compounds and complexes according to the invention lend themselves as (theragnostic) tracers, imaging agents and therapeutic agents for detecting tumor antigen-expressing target cells and tissues and treating and diagnosing cancer, such as PSMA-expressing target cells and tissues in a PSMA-related cancer, e.g. prostate cancer.

Prostate cancer continues to be the most prevalent cancer type in men and the third leading cause of cancer deaths in the western world (Ferlay, J.; Steliarova-Foucher, E.; Lortet-Tieulent, J.; Rosso, S.; Coebergh, J. W.; Comber, H.; Forman, D.; Bray, F. Cancer incidence and mortality patterns in Europe: estimates for 40 countries in 2012. Eur J Cancer 2013, 49, (6), 1374-403; Miller, K. D.; Siegel, R. L.; Lin, C. C.; Mariotto, A. B.; Kramer, J. L.; Rowland, J. H.; Stein, K. D.; Alteri, R.; Jemal, A. Cancer treatment and survivorship statistics, 2016. CA Cancer J Clin 2016, 66, (4), 271-89). At least 1-2 million men in the western hemisphere suffer from prostate cancer and it is estimated that the disease will strike one in six men between the ages of 55 and 85. According to the American Cancer Society, approximately 161,000 new cases of prostate cancer are diagnosed each year in USA. The 5-year survival rate of patients with stage IV metastatic prostate cancers is only about 29%. The treatment of metastatic castration-resistant prostate cancer (mCRPC) remains difficult and options to cure patients that reached this stage of the disease do not exist. The development of new concepts for an effective therapy is, therefore, urgently needed.

Once a metastatic prostate cancer becomes hormone-refractory there are only a few therapy options left, often with rather poor clinical success. According to the current medical guidelines, antimitotic chemotherapy with docetaxel is typically recommended. However, treatment is often associated with severe side effects, and only marginally improved survival rates. Early diagnosis and close monitoring of potential relapses are therefore crucial. Prostate cancer diagnosis is based on examination of histopathological or cytological specimens from the gland. Existing imaging techniques for therapeutic monitoring of progressing or recurring prostate cancer, include computed tomography (CT), magnetic resonance (MR) imaging and ultrasound, but are often insufficient for effective monitoring and management of the disease. Consequently, there is a high clinical demand for more effective tools for both early diagnosis and treatment of prostate cancer.

It is well known that tumor cells may express unique proteins exhibiting a modified structure due to mutation, or may over-express normal (i.e. non-mutated) proteins that are normally produced in extremely small quantities in non-malignant cells. Tumor antigens may be broadly classified into two categories based on their expression pattern: Tumor-Specific Antigens (TSA), which are present only on tumor cells and not on non-malignant cells and Tumor-Associated Antigens (TAA), which are present on some tumor cells and also non-malignant cells. TSAs typically emerge as a result of the mutation of protooncogenes and tumor suppressors which lead to abnormal protein production, whereas TAA expression is generally caused by mutation of other genes unrelated to the tumor formation.

The expression of such proteins on the surface of tumor cells offers the opportunity to diagnose and characterize disease by detecting such tumor markers. Proteinaceous binding agents or small molecule drugs carrying visualizable labels and specifically recognizing such tumor markers are typically employed for diagnosing and imaging cancers under non-invasive conditions.

A promising new series of low molecular-weight imaging agents targets the prostate-specific membrane antigen (PSMA). PSMA, also known as folate hydrolase I (FOLH1), is a transmembrane, 750 amino acid type II glycoprotein. The PSMA gene is located on the short arm of chromosome 11 and functions both as a folate hydrolase and neuropeptidase. It has neuropeptidase function that is equivalent to glutamate carboxypeptidase II (GCPII), which is referred to as the “brain PSMA”, and may modulate glutamatergic transmission by cleaving N-acetyl-aspartyl-glutamate (NAAG) to N-acetylaspartate (NAA) and glutamate (Nan, F.; et al. J Med Chem 2000, 43, 772-774).

The prostate-specific membrane antigen (PSMA) is overexpressed in the majority of prostate cancer cases (Silver, D. A.; Pellicer, I.; Fair, W. R.; Heston, W. D.; Cordon-Cardo, C. Prostate-specific membrane antigen expression in normal and malignant human tissues. Clin Cancer Res 1997, 3, (1), 81-5; Cunha, A. C.; Weigle, B.; Kiessling, A.; Bachmann, M.; Roeber, E. P. Tissue-specificity of prostate specific antigens: comparative analysis of transcript levels in prostate and non-prostatic tissues. Cancer Lett 2006, 236, (2), 229-38). It emerged, therefore, as a promising target for nuclear imaging and radionuclide therapy of mCRPC (Bouchelouche, K.; Choyke, P. L. Prostate-specific membrane antigen positron emission tomography in prostate cancer: a step toward personalized medicine. Curr Opin Oncol 2016, 28, (3), 216-21; Haberkorn, U.; Eder, M.; Kopka, K.; Babich, J. W.; Eisenhut, M. New strategies in prostate cancer: prostate-specific membrane antigen (PSMA) ligands for diagnosis and therapy. Clin Cancer Res 2016, 22, (1), 9-15; Eiber, M.; Fendler, W. P.; Rowe, S. P.; Calais, J.; Hofman, M. S.; Maurer, T.; Schwarzenboeck, S. M.; Kratowchil, C.; Herrmann, K.; Giesel, F. L. Prostate-specific membrane antigen ligands for imaging and therapy. J Nucl Med 2017, 58, (Suppl 2), 67S-76S). PSMA is (i) mainly restricted to the prostate (although is also detected in lower amounts in the neovasculature of numerous other solid tumors, including bladder, pancreas, lung, and kidney cancers, but not in normal vasculature), (ii) abundantly expressed as protein at all stages of prostate cancer (in amounts of up to 10⁶ PSMA molecules per cancer cell) (iii) presented at the cell surface but not shed into the circulation, and (iv) associated with enzymatic or signaling activity. Moreover, PSMA expression is further up-regulated in poorly differentiated, androgen-insensitive or metastatic cancers and the expression usually correlateds with disease progression.

The unique expression of PSMA makes it an important marker of prostate cancer (and a few other cancers as well). Furthermore, PSMA represents a large extracellular target for imaging agents. PSMA is internalized after ligand binding and, thus, it is not only an excellent target for targeted radionuclide therapy (using particle-emitting radionuclides) but also for other therapeutic strategies including the tumor cell-specific delivery of immunotoxins, retargeting of immune cells, pro-drug activation, PSMA vaccines, and plasmid DNA and adenoviral immunizations. Because of low expression levels in healthy tissue, PSMA has additionally the potential for high-dose therapy, with minimized side effects.

In the past, several PSMA-targeting agents carrying therapeutic or diagnostic moieties were developed. The FDA-approved radio-immunoconjugate of the anti-PSMA monoclonal antibody (mAb) 7E11, known as PROSTASCINT®, has been used to diagnose prostate cancer metastasis and recurrence. The success of this radiopharmaceutical agent is limited due to the fact that this antibody binds to the intracellular domain of PSMA, hence, can target only dead cells. Moreover, the use of monoclonal antibodies and antibody fragments as imaging agents is often limited due to their slow renal clearance, heterogenous distribution, poor tumor penetration and immunogenic potential.

In order to overcome these problems, various small-molecule PSMA targeting agents capable of binding to the extracellular domain of PSMA were developed for PET/CT and SPECT/CT imaging, including radiolabeled N—[N—[(S)-1,3-dicarboxypropyl]carbamoyl]-S-[11C]methyl-l-cysteine (DCFBC) and several urea-based peptidomimetic PSMA-inhibitors (cf. Bouchelouche et al. Discov Med. 2010 January; 9(44): 55-61), including MIP-1095 (Hillier et al. Cancer Res. 2009 Sep. 1; 69(17):6932-40), a PSMA ligand currently in clinical evaluation, and DOTA-conjugated PSMA-inhibitor PSMA-617 developed by Benesova et al (JNM 2015, 56: 914-920 and EP 2862 857 A1), which distributes throughout the body and rapidly clears from the blood (J Nucl Med. 2015; 56(11):1697-705). However, although rapid and systemic access advantageously facilitates tumor targeting and—penetration, currently available PSMA-targeting agents bear the risk of mediating unspecific “off-target” interactions in normal tissues expressing the target, and of accumulation of the radiopharmaceuticals in excretory organs (such as the kidneys). Thereby, non-tumorous tissues may be exposed to radiation doses ultimately leading to irreversible tissue damage. It was demonstrated that different radiolabeled small-molecule PSMA-targeting agents (including PSMA-617) accumulate in patients' lacrimal and salivary glands and may cause damage to the glandular tissue, especially if used in combination with alpha-emitting radionuclides (Zechmann et al. Eur J Nucl Med Mol Imaging. 2014; 41(7):1280-92 and Kratochwil et al. J Nucl Med. 2017 Apr. 13. pii: jnumed.117.191395. doi: 10.2967/jnumed.117.191395 [Epub]). One possible solution to that problem involves the use of PSMA-binding agents with a high-affinity towards PSMA (Kratochwil et al. J Nucl Med. 2015; 293-298 and Chatalic et al. Theragnostics. 2016; 6: 849-861).

Recently, the concept of modifying radiopharmaceuticals with an albumin-binding entity was applied to PSMA-targeting radioligands by various groups (Choy, C. J.; Ling, X.; Geruntho, J. J.; Beyer, S. K.; Latoche, J. D.; Langton-Webster, B.; Anderson, C. J.; Berkman, C. E. ¹⁷⁷Lu-Labeled phosphoramidate-based PSMA inhibitors: the effect of an albumin binder on biodistribution and therapeutic efficacy in prostate tumor-bearing mice. Theranostics 2017, 7, (7), 1928-1939; Kelly, J. M.; Amor-Coarasa, A.; Nikolopoulou, A.; Wustemann, T.; Barelli, P.; Kim, D.; Williams, C., Jr.; Zheng, X.; Bi, C.; Hu, B.; Warren, J. D.; Hage, D. S.; DiMagno, S. G.; Babich, J. W. Double targeting ligands with modulated pharmacokinetics for endoradiotherapy of prostate cancer. J Nucl Med 2017; Benesova, M.; Umbricht, C. A.; Schibli, R.; Müller, C. Albumin-binding PSMA ligands: optimization of the tissue distribution profile. Mol Pharm 2018, 15, (3), 934-946; Umbricht, C. A.; Benesova, M.; Schibli, R.; Müller, C. Preclinical development of novel PSMA-targeting radioligands: modulation of albumin-binding properties to improve prostate cancer therapy. Mol Pharm 2018, Mol Pharm 2018, 15, (6), 2297-2306). Indeed, such radioligands showed enhanced blood circulation and, thus, increased accumulation in the tumor tissue and better retention as compared to PSMA-binding radioligands without albumin-binding entity, such as ¹⁷⁷Lu-PSMA-617 (Benesova, M.; Umbricht, C. A.; Schibli, R.; Müller, C. Albumin-binding PSMA ligands: optimization of the tissue distribution profile. Mol Pharm 2018, 15, (3), 934-946; Umbricht, C. A.; Benesova, M.; Schibli, R.; Müller, C. Preclinical development of novel PSMA-targeting radioligands: modulation of albumin-binding properties to improve prostate cancer therapy. Mol Pharm 2018, Mol Pharm 2018, 15, (6), 2297-2306). The retention of radioactivity in the blood was high and, therefore, uptake in other organs and tissues, including the kidneys, was higher than in the case of PSMA-binding radioligands without albumin-binding entity, such as ¹⁷⁷Lu-PSMA-617 (Benesova, M.; Umbricht, C. A.; Schibli, R.; Müller, C. Albumin-binding PSMA ligands: optimization of the tissue distribution profile. Mol Pharm 2018, 15, (3), 934-946).

For example, Choy et al. Theranostics 2017; 7(7):1928-1939, evaluated ¹⁷⁷Lu-labeled phosphoramidate-based PSMA inhibitor with an albumin-binding entity. A DOTA chelator complexing the ¹⁷⁷Lu radionuclide was ether-linked to the irreversible PSMA inhibitor CTT1298 (EP 2970345 A1). Phosphoramidate-based PSMA binding motive, however, exhibits only poor stability, especially at elevated temperatures (elevated temperatures under extended acidic conditions lead to hydrolysis of phosphoramidate P—N bond), which are required for the coordinative radiolabeling reaction via chelators such as DOTA. Therefore a direct radiolabeling reaction cannot be applied and a multi-step pre-labeling approach has to be used. Thus, ¹⁷⁷Lu-DOTA-azide as precursor should be prepared; subsequently the precursor has to be coupled to a dibenzocyclooctyne-derivatized PSMA motive. Finally, elaborate HPLC purification of the coupled compound must be undertaken; reformulation with evaporation (under N₂ atmosphere) of the HPLC-eluent and dissolving in a physiological medium need to be performed. This procedure is likely not possible for a clinical application when high activities are being produced. Pre-clinical biodistribution data demonstrate poor performance of the radiolabeled agent especially regarding tumour-to-kidney ratios which did not exceed far above 1.

Another approach was followed by Kelly et al. (Kelly, J. M.; Amor-Coarasa, A.; Nikolopoulou, A.; Wustemann, T.; Barelli, P.; Kim, D.; Williams, C., Jr.; Zheng, X.; Bi, C.; Hu, B.; Warren, J. D.; Hage, D. S.; DiMagno, S. G.; Babich, J. W. Double targeting ligands with modulated pharmacokinetics for endoradiotherapy of prostate cancer. J Nucl Med 2017), who evaluated agents exhibiting affinity for both PSMA and for human serum albumin (HSA). The ligands developed by Kelly et al. comprise a p-(iodophenyl)butyric acid entity for HSA binding and an urea-based PSMA binding entity. In the compounds developed by Kelly et al., radiotherapeutic iodine (¹³¹I) is covalently attached to the HSA binding moiety, which is in turn directly connected to the PSMA binding entity via a hydrocarbyl chain. However, the evaluated compounds are considerably limited in terms of the applied radionuclide which is limited to iodine. Further, no improved internalization/uptake in target cells was demonstrated for the evaluated compounds.

The structural entity, (p-iodophenyl)butyric acid, was previously discovered to bind with high affinity to serum albumin (Dumelin, C. E.; Trüssel, S.; Buller, F.; Trachsel, E.; Bootz, F.; Zhang, Y.; Mannocci, L.; Beck, S. C.; Drumea-Mirancea, M.; Seeliger, M. W.; Baltes, C.; Muggler, T.; Kranz, F.; Rudin, M.; Melkko, S.; Scheuermann, J.; Neri, D. A portable albumin binder from a DNA-encoded chemical library. Angew Chem Int Ed Engl 2008, 47, (17), 3196-201). It was used for the modification of fast-cleared antibody fragments to increase their blood circulation time and, hence, improve the pharmacokinetics (Trüssel, S.; Dumelin, C.; Frey, K.; Villa, A.; Buller, F.; Neri, D. New strategy for the extension of the serum half-life of antibody fragments. Bioconjug Chem 2009, 20, (12), 2286-92). In the case of folate radioconjugates, the modification with this same albumin binder led to a significantly increased tumor uptake and reduced retention of radioactivity in the kidneys dramatically (Müller, C.; Struthers, H.; Winiger, C.; Zhernosekov, K.; Schibli, R. DOTA conjugate with an albumin-binding entity enables the first folic acid-targeted ¹⁷⁷Lu-radionuclide tumor therapy in mice. J Nucl Med 2013, 54, (1), 124-31; Siwowska, K.; Haller, S.; Bortoli, F.; Benesova, M.; Groehn, V.; Bernhardt, P.; Schibli, R.; Müller, C. Preclinical comparison of albumin-binding radiofolates: impact of linker entities on the in vitro and in vivo properties. Mol Pharm 2017, 14, (2), 523-532).

The albumin-binding properties of these PSMA-radioligands were more pronounced than previously seen with folate radioconjugates comprising the same p-iodophenyl-based albumin-binding entity. It was, therefore, speculated that a weaker binding of PSMA-ligands to serum albumin would be beneficial. In terms of radioligand design, this was addressed by substitution of the strong albumin binder (p-iodophenyl)butyric acid with (p-tolyl)butyric acid that was previously shown to exhibit reduced albumin-binding affinity albumin (Dumelin, C. E.; Trüssel, S.; Buller, F.; Trachsel, E.; Bootz, F.; Zhang, Y.; Mannocci, L.; Beck, S. C.; Drumea-Mirancea, M.; Seeliger, M. W.; Baltes, C.; Muggler, T.; Kranz, F.; Rudin, M.; Melkko, S.; Scheuermann, J.; Neri, D. A portable albumin binder from a DNA-encoded chemical library. Angew Chem Int Ed Engl 2008, 47, (17), 3196-201). Accordingly, ¹⁷⁷Lu-PSMA-ALB-56, a PSMA-binding radioligand equipped with a p-tolyl-moiety as albumin-binding entity instead of a p-iodophenyl-based albumin-binding entity, demonstrated more favorable tumor-to-background ratios than ¹⁷⁷Lu-PSMA-ALB-53 which was equipped with a p-iodophenyl moiety (Umbricht, C. A.; Benesova, M.; Schibli, R.; Müller, C. Preclinical development of novel PSMA-targeting radioligands: modulation of albumin-binding properties to improve prostate cancer therapy. Mol Pharm 2018, Mol Pharm 2018, 15, (6), 2297-2306). The blood activity levels were, however, still relatively high in the case of ¹⁷⁷Lu-PSMA-ALB-56 which may be an indication that the albumin-binding affinity was still too strong.

This shows the necessity of balancing the binding of the PSMA-binding radioligand to albumin in order to achieve an optimal tissue distribution profile with high tumor uptake, but blood activity levels that are not extensively high as it would comprise a risk for undesired side effects to healthy tissue.

Despite advances over the years, diagnosis and management of prostate cancer still remains challenging. New diagnostic or imaging agents capable of targeting cancer tumor cells in a highly selective manner and exhibiting favorable pharmacokinetic properties for rapid and non-invasive tumor visualization and therapy are needed to enable early detection and treatment of cancer.

It is thus an object of the present invention to overcome the disadvantages in the prior art and comply with the need in the art.

That object is solved by the subject-matter disclosed herein, more specifically as set out by the appended claims.

The invention provides a new class of PSMA-binding radioligands, which comprise ibuprofen as an albumin binding entity, a PSMA-binding moiety and chelator moiety, thus forming a trifunctional compound.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

In the present invention, if not otherwise indicated, different features of alternatives and embodiments may be combined with each other.

For the sake of clarity and readability the following definitions are provided. Any technical feature mentioned for these definitions may be read on each and every embodiment of the invention. Additional definitions and explanations may be specifically provided in the context of these embodiments.

### Definitions

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

The term “hydrocarbyl” refers to residues of hydrocarbon groups, i.e., hydrocarbon chain radicals, preferably independently selected from the group alkyl, alkenyl, alkynyl, aryl and aralkyl.

The term “alkyl” comprises linear (“straight-chain”), branched and cyclic chain radicals having 1-30 carbon atoms, preferably 1-20, 1-15, 1-10, 1-8, 1-6, 1-4, 1-3 or 1-2 carbon atoms. For instance, the term “C₁₋₁₂ alkyl” refers to a hydrocarbon radical whose carbon chain is straight-chain or branched or cyclic and comprises 1 to 12 carbon atoms. Specific examples for alkyl residues are methyl, ethyl, propyl, isopropyl, butyl, pentyl, hexyl, octyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, pentacosyl, hexacosyl, heptacosyl, octacosyl, nonacosyl or triacosyl, including the various branched-chain and/or cyclic isomers thereof, e.g. isobutyl, tert.-butyl or isopentyl. Cyclic alkyl isomers are also referred to as “cycloalkyl” herein to refer to saturated alicyclic hydrocarbons comprising 3 ring carbon atoms. “Substituted” linear, branched and cyclic alkyl groups are generally also encompassed by the term. The term further includes “heteroalkyl”, referring to alkyl groups wherein one or more C-atoms of the carbon chain are replaced with a heteroatom such as, but not limited to, N, O, and S. Accordingly, the term further includes “heterocyclyl” or “heterocycloalkyl”, referring to non-aromatic ring compounds containing 3 or more ring members, of which one or more ring carbon atoms are replaced with a heteroatom such as, but not limited to, N, O, and S. Heterocyclyl groups encompass unsaturated, partially saturated and saturated ring systems, such as, for example, imidazolyl, imidazolinyl and imidazolidinyl groups. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Heterocyclyl groups may be substituted or unsubstituted. Representative substituted heterocyclyl groups may be monosubstituted or substituted more than once, such as, but not limited to, pyridyl or morpholinyl groups, which are 2-, 3-, 4-, 5-, or 6-substituted, or disubstituted with various substituents such as those listed above.

The term “cyclic” includes the term “polycyclic”, referring to structures having more than one ring structure. In particular, the term “cyclic” also refers to spirocyclic structures, wherein two or more rings have one atom in common, and 5 fused polycyclic structures, wherein two or more rings have at least two atoms in common.

The term “alkenyl” as used herein comprises linear, branched and cyclic chain 10 radicals having 2-30 carbon atoms, preferably 2-20, 2-15, 2-10, 2-8, 2-6, 2-4, or 2-3 carbon atoms, including at least one carbon-to-carbon double bond. Specific examples of “alkenyl” groups are the various alkenic unsaturated equivalents of those given with respect to alkyl groups, named after the conventions known to the person skilled in the art, depending on the number and location of carbon-to-carbon double bond or bonds, e.g. butanediylidene, 1-propanyl-3-ylidene. “Alkenyl” groups preferably contain at least 1, more preferably at least 2, 3, 4, 6, 7, 8, 9, 10, 11, 12, 13, 14, 15, or 16 double bonds, wherein a double bond is preferably located at position 1, 2, 3, 4, 5, 6, 7, 8, 9, 10, 12, 13, 14, 15, 16, 17, 18, 19, 20, 21, 22, 23, 24, 25, 26, 27, 28 or 29 of the hydrocarbyl chain. Alkenyl groups may be substituted or unsubstituted.

The term “alkynyl” as employed herein comprises straight, branched and cyclic chain radicals having 2-30 carbon atoms, preferably 2-20, 2-15, 2-10, 2-8, 2-6, 2-4, or 2-3 carbon atoms, including at least one carbon-to-carbon triple bond. Specific examples of “alkynyl” groups are the various alkynic unsaturated equivalents of those given with respect to alkyl and alkenyl groups, named after the conventions known to the person skilled in the art, depending on the number and location of carbon-to-carbon triple bond or bonds. “Alkynyl” groups preferably contain at least 1, more preferably at least 2, 3, 4, 6, 7, 8, 9, 10, 11, 12, 13, 14, 15, or 16 triple bonds, wherein a double triple bond is preferably located at position 1, 2, 3, 4, 5, 6, 7, 8, 9, 10, 12, 13, 14, 15, 16, 17, 30 18, 19, 20, 21, 22, 23, 24, 25, 26, 27, 28 or 29 of the hydrocarbyl chain. Alkynyl groups may be substituted or unsubstituted.

The term “aryl” refers to monocyclic or polycyclic or fused polycyclic aromatic ring systems.

The term includes monocyclic or polycyclic or fused polycyclic aromatic “heteroaryl” ring systems wherein at least one carbon atom of the ring system is substituted by a heteroatom. Typically, the terms “aryl” and “heteroaryl” refers to groups having 3-30 carbon atoms., such as 3-10, in particular 2-6 carbon atoms.

The terms “arylalkyl” or “aralkyl” are used interchangeably herein to refer to groups comprising at least one alkyl group and at least one aryl group as defined herein. In an aralkyl group as defined herein, the aralkyl group is bonded to another moiety of the compounds or conjugates of the invention via the alkyl group as exemplified by a benzyl group.

The term “halogen” or “halo” as used herein includes fluoro (F), chloro (Cl), bromo (Br), iodo (I).

The term “heteroatom” includes N, O, S and P, preferably N and O.

The term “substituted” refers to a hydrocarbyl group, as defined herein (e.g., an alkyl or alkenyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a “substituted” group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN), haloalkyl; aminoalkyl; hydroxyalkyl; and cycloalkyl.

### Compounds

In a first aspect the present invention provides a compound according to General Formula (1)(i) or (1)(ii):

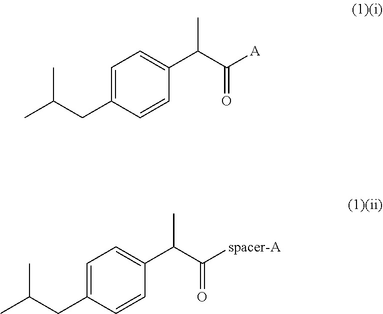

wherein A is a diagnostic or therapeutic agent comprising a binding site for a tumor antigen, and the spacer comprises at least one C—N bond.

Accordingly, the present invention provides plasma protein-binding tumor antigen ligands (in particular plasma protein-binding PSMA ligands) with favorable pharmacokinetic profiles. As used herein, the term “pharmacokinetics” preferably includes the stability, bioavailability, absorption, biodistribution, biological half-life and/or clearance of a therapeutic or diagnostic agent in a subject.

In the prior art, albumin binding entities were employed in order to extend circulation half-life of conjugates, to effect compartmentalization of conjugates in the blood and to improve delivery to the tumor antigen-expressing (tumor) target cells or tissues, resulting in increased tumor:non-target ratios for tumor antigen expressing normal (non-tumorous) organs. Accordingly, without being bound to any theory, it is assumed that the albumin binding entity confers improved pharmacokinetic properties to conjugate. However, prior art albumin binding entities useful in conjugates may result in a pronounced background signal (and, thus, unfavorable tumor-to-background ratios).

The aim of this study was, therefore, to replace the previously-used albumin binders by a different albumin binding entity to find an optimum between albumin-binding properties and clearance of the conjugate (and, e.g., its radioactivity) from background tissues and organs. The present inventors surprisingly found that ibuprofen as albumin-binding entity in tumor-antigen-binding radioligands achieved such a desired balance between plasma protein-binding properties and clearance of radioactivity from background tissues and organs. This was in particular surprising, as it was previously assumed that ibuprofen loses its albumin-binding affinity as soon as one attempts to modify the carboxylic acid group of the molecule in order to couple ibuprofen to other moieties of biopharmaceutical interest (WO 2008/053360 A2; US 2010/172844; Dumelin, C. E.; Trüssel, S.; Buller, F.; Trachsel, E.; Bootz, F.; Zhang, Y.; Mannocci, L.; Beck, S. C.; Drumea-Mirancea, M.; Seeliger, M. W.; Baltes, C.; Muggler, T.; Kranz, F.; Rudin, M.; Melkko, S.; Scheuermann, J.; Neri, D. A portable albumin binder from a DNA-encoded chemical library. Angew Chem Int Ed Engl 2008, 47, (17), 3196-201). Despite this technical prejudice, the present inventors found surprisingly that a balanced binding to albumin can be achieved by coupling ibuprofen via its carboxylic acid group to a diagnostic or therapeutic agent comprising a binding site for a tumor antigen.

Albumin, in particular human serum albumin (HSA), is the most abundant protein in (human) plasma and constitutes about half of serum protein. The term “human serum albumin” or “HSA” as used herein preferably refers to the serum albumin protein encoded by the human ALB gene. More preferably, the term refers to the protein as characterized under UniProt Acc. No. P02768 (entry version 240, last modified May 10, 2017, or functional variants, isoforms, fragments or (post-translationally or otherwise modified) derivatives thereof.

The diagnostic or therapeutic agent A, as used herein, may be any agent useful in diagnosis, prevention or therapy of a disease (in particular cancer) as long as it comprises a binding site for a tumor antigen.

Tumor antigens are proteins expressed by tumor cells, which may exhibit a modified structure due to mutation, or which may over-express in comparison to normal (i.e. non-mutated) proteins that are normally produced in extremely small quantities in non-malignant cells. Tumor antigens may be broadly classified into two categories based on their expression pattern: Tumor-Specific Antigens (TSA), which are present only on tumor cells and not on non-malignant cells and Tumor-Associated Antigens (TAA), which are present on some tumor cells and also non-malignant cells. TSAs typically emerge as a result of the mutation of protooncogenes and tumor suppressors which lead to abnormal protein production, whereas TAA expression is generally caused by mutation of other genes unrelated to the tumor formation. Preferably, the tumor antigen is prostate-specific membrane antigen (PSMA). Accordingly, it is preferred that the diagnostic or therapeutic agent A comprises a binding site for PSMA.

In addition to the binding site for a tumor antigen, the diagnostic or therapeutic agent A may comprise further components, such as a (further) active component (for diagnosis, prevention or therapy of a disease such as cancer) and/or one or more linker(s). One or more “linker” or “spacer” may be used to combine various components, such as the tumor-antigen binding entity, one or more further active component(s) and, optionally, the ibuprofen as albumin-binding entity in one single molecule. For example, the tumor antigen binding entity, such as a PSMA binding entity (e.g., as described herein), may coupled to a linker as described herein. For example, ibuprofen may be coupled to a spacer as described herein.

Preferably, the diagnostic or therapeutic agent A comprises a radiolabel. As used herein, the term “radiolabel” (or radioactive tracer) refers to a radioactive label, such as a radioactive substance or a radioactive atom (e.g., a radionuclide). For example, the radiolabel may be a non-metallic radionuclide or a radiometal. While non-metallic radionuclides such as ¹⁸F, ¹¹C, ¹³N, ¹⁵O, or ¹²⁴I can be linked covalently to an organic molecule, radiometals such as ^{99m}Tc, ^{67/68}Ga, ¹¹¹In, or ¹⁷⁷Lu usually need to be coordinated via a so-called “chelator”. Accordingly, in particular if the diagnostic or therapeutic agent A comprises a radiometal as radiolabel, it is preferred that the diagnostic or therapeutic agent A comprises a chelator. The chelator may be conjugated to the other components of the diagnostic or therapeutic agent A (such as to the tumor-antigen binding site and/or to the ibuprofen) via a linker. For example, diagnostic or therapeutic agent A may comprise a radiometal coordinated via the chelator. Preferably, the chelator is conjugated to the other components of the diagnostic or therapeutic agent A (such as to the tumor-antigen binding site and/or to the ibuprofen) via a linker.

As used herein, the terms “tumor antigen ligand” (e.g., “PSMA ligand”), “compound” and “conjugate” are used interchangeably and refer to the complete molecule (including at least a tumor antigen binding site and ibuprofen and, optionally, further components).

In particular, the tumor antigen ligands (such as PSMA ligands) according to the invention (also referred to as “conjugates” or “compounds” herein) may include:
a first terminal group (a chelator, e.g. for coordination with a radiometal or coordinated with a radiometal),
a second terminal group (ibuprofen as albumin binding entity), and
a third terminal group (a tumor antigen binding entity, such as a PSMA binding entity) that are covalently connected or linked to each other via appropriate linkers or spacers.

Accordingly, the present invention provides a compound of General Formula (1)(a):

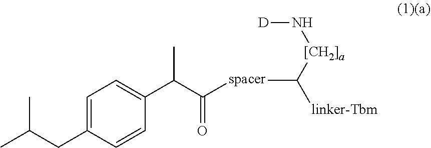

wherein D is a chelator;
   Tbm is a tumor-antigen binding moiety (also referred to as tumor-antigen binding entity);
   linker is a linker, preferably comprising a cyclic group or an aromatic group;
   spacer is a spacer comprising a C—N bond; and
   a is an integer selected from 0, 1, 2, 3, 4, 5, 6, 7, 8, 9, or 10, preferably 0 or 1;
      or a pharmaceutically acceptable salt, ester, solvate or radiolabeled complex thereof.

In General Formula (1)(a), the three terminal groups (ibuprofen, the chelator (D) and the tumor-antigen binding moiety (Tbm) are connected via a linker and a spacer as shown in General Formula (1)(a) in a “branching point” (a CH-group):

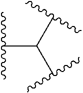

The position of the “branching point” (CH-group) in Formula (1)(a) is indicated below by the arrow:

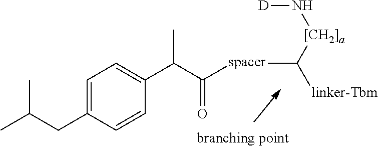

The chelator D, the tumor-antigen binding moiety Tbm, the linker and the spacer are preferably defined as described herein.

The tumor-antigen binding moiety (Tbm) is in particular a PSMA-binding moiety (Pbm).

Preferably, a is selected from 0, 1, 2, 3, 4, or 5; more preferably from 0, 1, or 2; and most preferably a is 0.

It is particularly envisaged that the structure included in the dashed line in Formula (1)(a) below comprises at least one peptide bond:

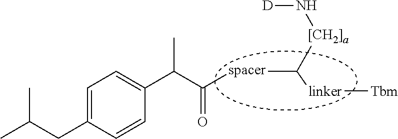

The inventive conjugates are ligands exhibiting affinity towards both, a tumor antigen (such as PSMA) and HSA. The term “ligand” as used herein refers to a compound capable of interacting with (targeting, binding to) a target (here: HSA or a tumor antigen, e.g. PSMA). The inventive conjugates may also be defined functionally as “tumor antigen targeting agents” (such as “PSMA targeting agents”). Preferably, “ligands” are capable of selectively binding to their target. The term “selectively binding” means that a compound binds with a greater affinity to its intended target than it binds to another, non-target entity.

“Binding affinity” is the strength of the binding interaction between a ligand (e.g. a small organic molecule, protein or nucleic acid) to its target/binding partner. Binding affinity is typically measured and reported by the equilibrium dissociation constant (K_{D}), a ratio of the “off-rate” (k_{off}) and the “on-rate” (kₒₙ), which is used to evaluate and rank order strengths of bimolecular interactions. The “on-rate” (Kₒₙ) characterizes how quickly a ligand binds to its target, the “off-rate” (K_{off}) characterizes how quickly a ligand dissociates from its target. K_{D} (K_{off}/Kₒₙ) and binding affinity are inversely related. Thus, the term “selectively binding” preferably means that a ligand binds to its intended target with a K_{D} that is lower than the K_{D} of its binding to another, non-target entity. There are many ways to measure binding affinity and dissociation constants, such as ELISA, gel-shift assays, pull-down assays, equilibrium dialysis, analytical ultracentrifugation, surface plasmon resonance, and spectroscopic assays.

In the context of the present invention, the K_{D} for binding of the tumor antigen binding entity, such as a PSMA binding entity, to a non-target entity may be at least 1.5-fold, preferably at least 2-, 3-, 5-, 10-, 15-, 20-, 25-, 30-, 35-, 40-, 45-, 50-, 60-, 70-, 80-, 90-, 100-200-, 300-, 400-, 500-, 750-, or 1000-fold the K_{D} for binding of said conjugate or moiety to a tumor antigen, e.g. human PSMA. Similarly, the K_{D} for binding of the HSA binding entity to a non-target entity may be at least 1.5-fold, preferably at least 2-, 3-, 5-, 10-, 15-, 20-, 25-, 30-, 35-, 40-, 45-, 50-, 60-, 70-, 80-, 90-, 100-200-, 300-, 400-, 500-, 750-, or 1000-fold the K_{D} for binding of said conjugate or moiety to HSA.

In the context of the present invention, the conjugates may bind to the tumor antigen (e.g. PSMA) with higher binding affinity than to albumin (HSA). For example, the conjugates may bind to PSMA with high binding affinity with K_{D} values in the nanomolar (nM) range and with moderate affinity to HSA in the micromolar range (μM (micromolar)).

Specifically, it may be preferred to balance the PSMA and HSA-binding affinities so as to increase tumor uptake, while reducing potentially damaging off-target effects. In particular, the inventive conjugates may exhibit a higher binding affinity towards PSMA than towards HSA.

### PSMA Binding Moiety

The inventive conjugates comprise a tumor antigen binding site (tumor antigen binding moiety, Tbm), which is preferably a PSMA binding moiety (also referred to as “PSMA binding entity”). The PSMA binding moiety is preferably capable of selectively binding to human PSMA. The term “selectively binding” is defined above.

The PSMA binding entity may bind reversibly or irreversibly to PSMA, typically with a binding affinity less than about 100 μM (micromolar).

Human Prostate-specific membrane antigen (PSMA) (also referred to as glutamate carboxypeptidase II (GCPII), folate hydrolase 1, folypoly-gamma-glutamate carboxypeptidase (FGCP), and N-acetylated-alpha-linked acidic dipeptidase I (NAALADase I)) is a type II transmembrane zinc metallopeptidase that is most highly expressed in the nervous system, prostate, kidney, and small intestine. It is considered a tumor marker in prostate cancer. The term “Human Prostate-specific membrane antigen” or “PSMA” as used herein preferably refers to the protein encoded by the human FOLH1 gene. More preferably, the term refers to the protein as characterized under UniProt Acc. No. Q04609 (entry version 186, last modified May 10, 2017, or functional variants, isoforms, fragments or (post-translationally or otherwise modified) derivatives thereof.

The PSMA-binding entity may generally be a binding entity capable of selectively (and optionally irreversibly) binding to (human) Prostate-Specific Membrane Antigen (cf. Chang Rev Urol. 2004; 6(Suppl 10): S13-S18).

The PSMA binding entity is preferably chosen by its ability to confer selective affinity towards PSMA. Preferred PSMA binding moieties are described in WO 2013/022797 A1, WO 2015/055318 A1 and EP 2862857 A1, which are incorporated by reference in their entirety herein.

Accordingly, in the conjugate of the present invention, the PSMA-binding moiety may be characterized by General Formula (3), (3)′, (3)″ or (3)′″:

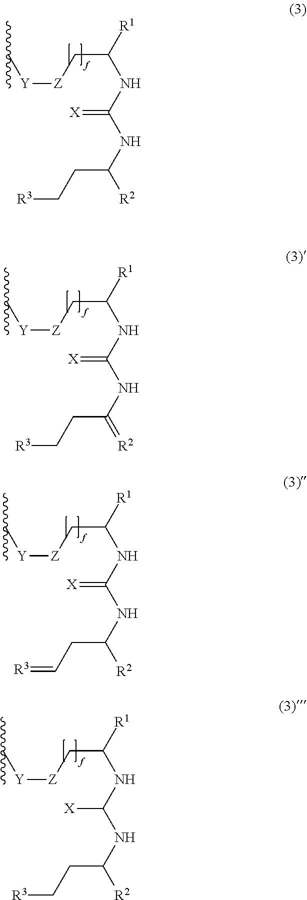

wherein
X and Y are each independently selected from O, N or NH or NH₂, S or P,
Z is selected from substituted or non-substituted CH₂,
R¹, R² and R³ are each independently selected from —COH, —CO₂H, —SO₂H, —SO₃H, —SO₄H, —PO₂H, —PO₃H, —PO₄H₂, —C(O)—(C₁-C₁₀)alkyl, —C(O)—O(C₁-C₁₀)alkyl, —C(O)—NHR⁴, or —C(O)—NR⁴R⁵, wherein R⁴ and R⁵ are each independently selected from H, bond, (C1-C10)alkylene, F, Cl, Br, I, C(O) or —CH(O), C(S) or —CH(S), —C(S)—NH-benzyl-, —C(O)—NH-benzyl, —C(O)—(C₁-C₁₀)alkylene, —(CH₂)ₚ—NH, —(CH₂)ₚ—(C₁-C₁₀)alkyene, —(CH₂)ₚ—NH—C(O)—(CH₂)_{q}, —(CHᵣCH₂)ₜNH—C(O)—(CH₂)ₚ, —(CH₂)ₚ—CO—COH, —(CH₂)ₚ—CO—CO₂H, —(CH2)ₚ—C(O)NH—C[(CH₂)_{q}—COH]₃, —C[(CH₂)ₚ—COH]₃, —(CH2)ₚ—C(O)NH—C[(CH₂)_{q}—CO₂H]₃, —C[(CH₂)ₚ—CO₂H]₃ or —(CH₂)ₚ—(C₅-C₁₄)heteroaryl, and
f, p, q, r and t are each independently an integer selected from 0, 1, 2, 3, 4, 5, 6, 7, 8, 9, or 10.

For the above referenced General Formula: (3)(i)′, (3)(ii)″ or (3)(iii)′″: R²-((3)′) or R³ ((3)″) are linked via double bonds. In Formula (3)′″ X is linked via a single bond.

With regard to X and Y it is understood that O, N, S or P may include hydrogen atoms, if appropriate. For example, Y may be O or NH.

Preferably, f is an integer selected from 1, 2, 3, 4, or 5; more preferably f is 2 or 3.

As outlined above, Z is selected from substituted or non-substituted CH₂. In other words, Z is selected from CH₂ or substituted CH₂, wherein one or both of the hydrogen atoms may be substituted. For example, Z is CH₂ or C═O.

Preferably, Y is NH and Z is CH₂. Accordingly, the PSMA-binding moiety may be characterized by General Formula (3)(ii):

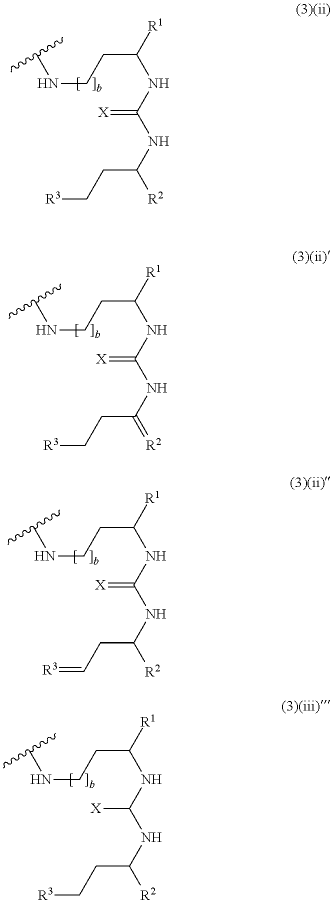

wherein
X is selected from O, N or NH or NH₂, S or P,
R¹, R² and R³ are each independently selected from —COH, —CO₂H, —SO₂H, —SO₃H, —SO₄H, —PO₂H, —PO₃H, —PO₄H₂, —C(O)—(C₁-C₁₀)alkyl, —C(O)—O(C₁-C₁₀)alkyl, —C(O)—NHR⁴, or —C(O)—NR⁴R⁵, wherein R⁴ and R⁵ are each independently selected from H, bond, (C1-C10)alkylene, F, Cl, Br, I, C(O) or —CH(O), C(S) or —CH(S), —C(S)—NH-benzyl-, —C(O)—NH-benzyl, —C(O)—(C₁-C₁₀)alkylene, —(CH₂)ₚ—NH, —(CH₂)ₚ—(C₁-C₁₀)alkyene, —(CH₂)ₚ—NH—C(O)—(CH₂)_{q}—(CHᵣCH₂)ₜ—NH—C(O)—(CH₂)ₚ, —(CH₂)ₚ—CO—COH, —(CH₂)ₚ—CO—CO₂H, —(CH2)ₚ—C(O)NH—C[(CH₂)_{q}—COH]₃, —C[(CH₂)ₚ—COH]₃, —(CH2)ₚ—C(O)NH—C[(CH₂)_{q}—CO₂H]₃, —C[(CH₂)ₚ—CO₂H]₃ or —(CH₂)ₚ—(C₅-C₁₄)heteroaryl, and
b, p, q, r and t are each independently an integer selected from 0, 1, 2, 3, 4, 5, 6, 7, 8, 9, or 10.

For the above referenced General Formula: (3)(i)′, (3)(ii)″ or (3)(iii)′″: R²-((3)′) or R³ ((3)″) are linked via double bonds. In Formula (3)′″ X is linked via a single bond.

In General Formulas (3) and (3)(ii) X is preferably O.

Moreover, it is preferred in General Formulas (3) and (3)(ii) that R¹, R² and R³ are each independently selected from —COH, —CO₂H, —SO₂H, —SO₃H, —SO₄H, —PO₂H, —PO₃H, —PO₄H₂. More preferably, in General Formulas (3) and (3)(ii) each of R¹, R² and R³ is —COOH.

In General Formula (3)(ii), b is preferably an integer selected from 1, 2, 3, 4 or 5, more preferably b is 2, 3 or 4, and most preferably b is 3.

It is also preferred in General Formula (3)(ii) that R¹, R² and R³ are each COOH, X is O, and b is 3.

Accordingly, the PSMA-binding moiety is most preferably characterized by Formula (3)(a):

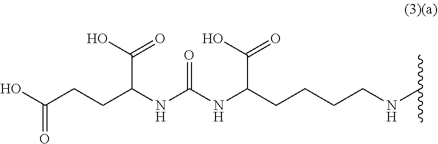 [SMILES: *NCCCCC(NC(=O)NC(CCC(=O)O)C(=O)O)C(=O)O]

As another specific example, the PSMA-binding moiety may also be characterized by Formula (3)(b):

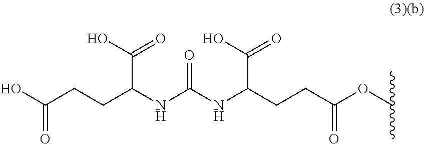 [SMILES: *OC(=O)CCC(NC(=O)NC(CCC(=O)O)C(=O)O)C(=O)O]

In view of the above, the present invention also provides a compound characterized by General Formula (1)(d):

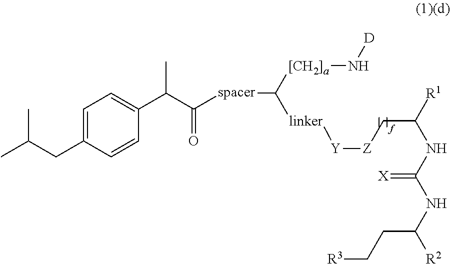

wherein D, the spacer, the linker and a are as defined herein for General Formula (1)(a) (and, preferably, its embodiments) and X, Y, Z, R¹, R², R³ and f are as defined herein for General Formula (3) (and, preferably, its embodiments),
   or a pharmaceutically acceptable salt, ester, solvate or radiolabeled complex thereof.

In particular, the present invention also provides a compound characterized by General Formula (1)(e):

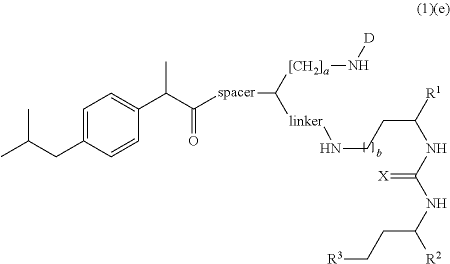

wherein D, the spacer, the linker and a are as defined herein for General Formula (1)(a) (and, preferably, its embodiments) and X, R¹, R², R³ and b are as defined herein for General Formula (3)(ii) (and, preferably, its embodiments),
   or a pharmaceutically acceptable salt, ester, solvate or radiolabeled complex thereof.

For example, the present invention also provides a compound characterized by General Formula (1)(f):

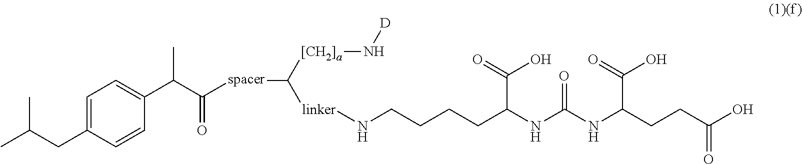

wherein D, the spacer, the linker and a are as defined herein for General Formula (1)(a) (and, preferably, its embodiments),
   or a pharmaceutically acceptable salt, ester, solvate or radiolabeled complex thereof.

### Linker

In the inventive conjugates, the tumor antigen binding moiety (e.g., PSMA binding entity) may be attached/connected to the “branching point” via a suitable linker. In the following, the term “linker” is used herein to specifically refer to the group connecting or linking and thus spanning the distance between the tumor antigen binding moiety (e.g., PSMA binding entity) and the —CH— “branching point”, and/or “spacing” the tumor antigen binding moiety (e.g., PSMA binding entity) apart from the remaining conjugate.

The linker may preferably avoid sterical hindrance between the tumor antigen binding moiety (e.g., PSMA binding entity) and the other groups or entities of the inventive conjugate and ensure sufficient mobility and flexibility. Further, the linker may preferably be designed so as to confer, support and/or allow sufficient HSA binding, high affinity tumor antigen (e.g., PSMA) binding, and rapid and optionally selective penetration of tumor antigen- (e.g., PSMA-) positive cells through internalization of the compound of the invention.

In particular PSMA binding entities, such as PSMA binding entities of General Formula (3) or (3)(ii), may preferably be linked to the inventive conjugate via a suitable linker as described, e.g. in EP 2 862 857 A1. Said linker may preferably confer optimized lipophilic properties to the inventive conjugate to increase PSMA binding and cellular uptake and internalization. The linker may preferably comprise at least one cyclic group and/or at least one aromatic group (in particular in group Q and W in General Formula (4) below).

Accordingly, in the inventive conjugates, a preferred linker may be characterized by General Formula (4):

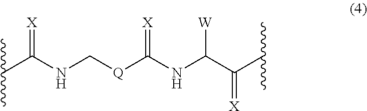

wherein
X is each independently selected from O, N, S or P,
Q is selected from substituted or unsubstituted alkyl, alkylaryl and cycloalkyl, preferably from substituted or unsubstituted C₅-C₁₄ aryl, C₅-C₄ alkylaryl or C₅-C₁₄ cycloalkyl, and
W is selected from —(CH₂)_{c}-aryl or —(CH₂)_{c}-heteroaryl, wherein c is an integer selected from 0, 1, 2, 3, 4, 5, 6, 7, 8, 9 or 10.

Without wishing to being bound to any theory, it is thought that hydrophilic or polar functional groups within or pendant from the linker (in particular Q, W) may advantageously enhance the PSMA-binding properties of the inventive conjugate.

Where Q is a substituted aryl, alkylaryl or cycloalkyl, exemplary substituents are listed in the “Definitions” section above and include, without limitation, halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN), haloalkyl, aminoalkyl, hydroxyalkyl, cycloalkyl.

Preferably, Q may be selected from substituted or unsubstituted C₅-C₇ cycloalkyl, more preferably, Q is cyclohexyl.

Preferably, W may be selected from —(CH₂)_{c}-naphthyl, —(CH₂)_{c}-phenyl, —(CH₂)_{c}-biphenyl, —(CH₂)_{c}-indolyl, —(CH₂)_{c}-benzothiazolyl, wherein c is an integer selected from 0, 1, 2, 3, 4, 5, 6, 7, 8, 9 or 10. More preferably, W may be selected from —(CH₂)-naphthyl, —(CH₂)-phenyl, —(CH₂)-biphenyl, —(CH₂)-indolyl or —(CH₂)-benzothiazolyl. Most preferably, W is —(CH₂)-naphthyl.

Preferably, each X may be O.

Accordingly, a particularly preferred linker connecting the tumor antigen binding moiety, in particular the PSMA binding entity, to the inventive conjugate may be characterized by the following Structural Formula (4)(a):

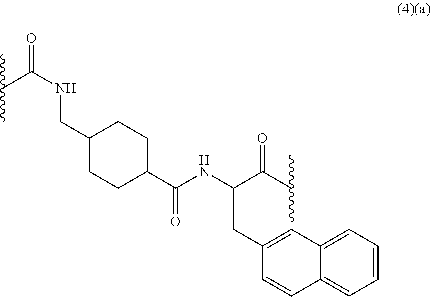 [SMILES: *C(=O)NCC1CCC(C(=O)NC(Cc2ccc3ccccc3c2)C(*)=O)CC1]

In view of the above, the present invention also provides a compound characterized by General Formula (1)(g):

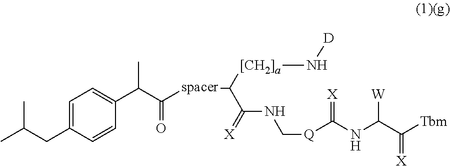

wherein D, Tbm, the spacer, and a are as defined herein for General Formula (1)(a) (and, preferably, its embodiments) and X, Q and W are as defined herein for General Formula (4) (and, preferably, its embodiments),
   or a pharmaceutically acceptable salt, ester, solvate or radiolabeled complex thereof.

For example, the present invention also provides a compound characterized by General Formula (1)(h):

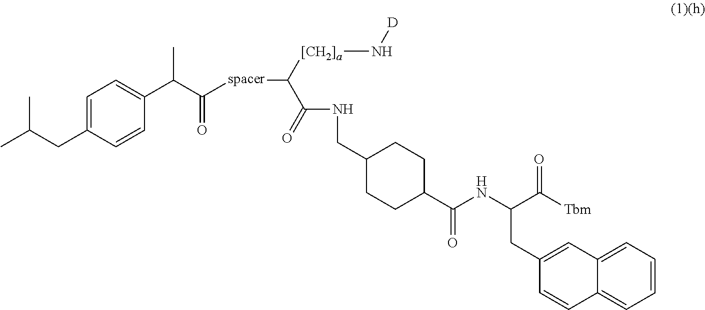

wherein D, Tbm, the spacer, and a are as defined herein for General Formula (1)(a) (and, preferably, its embodiments),
   or a pharmaceutically acceptable salt, ester, solvate or radiolabeled complex thereof.

In view of the above described embodiments for the specific tumor antigen binding moiety, namely the PSMA-binding moiety, and in view of the above described embodiments for the linker, the present invention also provides a compound characterized by General Formula (1)(k):

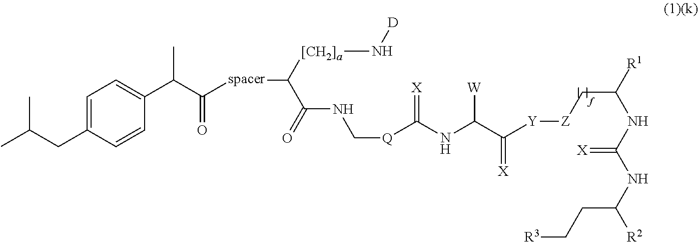

wherein D, the spacer, and a are as defined herein for General Formula (1)(a) (and, preferably, its embodiments); Y, Z, R¹, R², R³ and f are as defined herein for General Formula (3) (and, preferably, its embodiments), and X, Q and W are as defined herein for General Formula (4) (and, preferably, its embodiments),
   or a pharmaceutically acceptable salt, ester, solvate or radiolabeled complex thereof.

In particular, the present invention also provides a compound characterized by General Formula (1)(l):

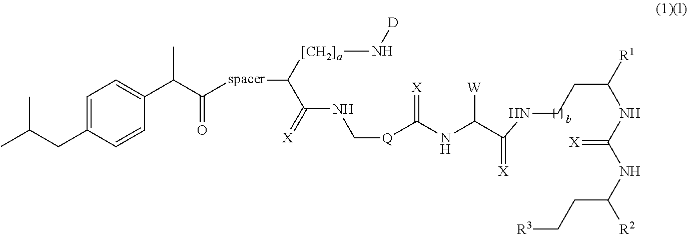

wherein D, the spacer, and a are as defined herein for General Formula (1)(a) (and, preferably, its embodiments); R¹, R², R³ and b are as defined herein for General Formula (3)(ii) (and, preferably, its embodiments), and X, Q and W are as defined herein for General Formula (4) (and, preferably, its embodiments),
   or a pharmaceutically acceptable salt, ester, solvate or radiolabeled complex thereof.

In particular, the present invention also provides a compound characterized by General Formula (1)(m):

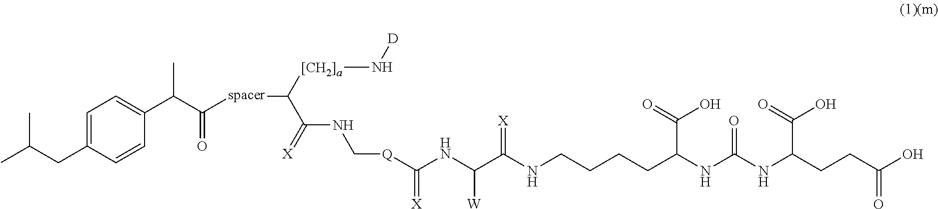

wherein D, the spacer, and a are as defined herein for General Formula (1)(a) (and, preferably, its embodiments); R¹, R², R³ and b are as defined herein for General Formula (3)(ii) (and, preferably, its embodiments), and X, Q and W are as defined herein for General Formula (4) (and, preferably, its embodiments),
   or a pharmaceutically acceptable salt, ester, solvate or radiolabeled complex thereof.

For example, the present invention also provides a compound characterized by General Formula (1)(b):

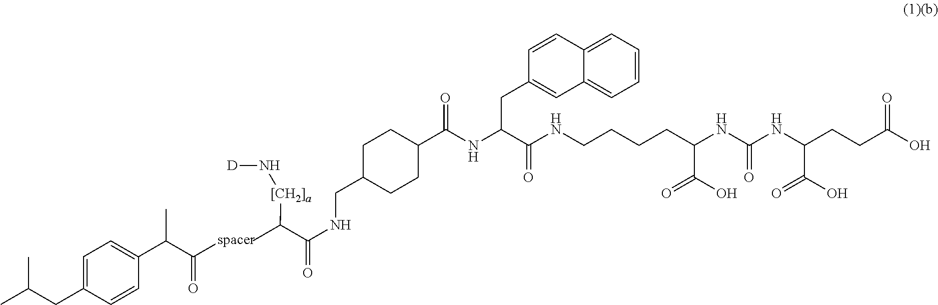

wherein D, the spacer, and a are as defined herein for General Formula (1)(a) (and, preferably, its embodiments),
   or a pharmaceutically acceptable salt, ester, solvate or radiolabeled complex thereof.

Even more specifically, the present invention also provides a compound characterized by General Formula (1)(c):

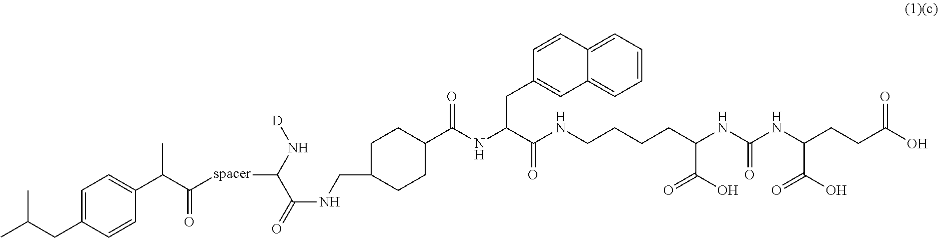

wherein D and the spacer are as defined herein for General Formula (1)(a) (and, preferably, its embodiments),
   or a pharmaceutically acceptable salt, ester, solvate or radiolabeled complex thereof.

### Spacer

In the inventive conjugates, ibuprofen (as albumin binding entity) is conjugated (i.e. covalently linked or attached to) to the —CH— “branching point” via a “spacer”. In the following, the term “spacer” is used herein to specifically refer to the group connecting and spanning the distance between the albumin binding entity and the —CH— “branching point”, and/or “spacing” these groups apart from the remaining groups/entities of the conjugate.

The spacer may preferably avoid sterical hindrance between the ibuprofen (as albumin binding entity) and the other groups or entities of the inventive conjugate and ensure sufficient mobility and flexibility. Further, the spacer may preferably be designed so as to confer, support and/or allow sufficient HSA binding, high affinity tumor antigen (e.g., PSMA) binding, and rapid and optionally selective penetration of tumor antigen- (e.g., PSMA-) positive cells through internalization of the compound of the invention.

The present inventors determined that the spacer should preferably comprise at least one C—N bond. Suitable spacers should preferably be stable in vivo. Spacer design may typically depend on the overall conjugate and may preferably be chosen to promote the functionality of the remaining conjugate (e.g. tumor antigen binding (such as PSMA binding), HSA binding, internalization etc.). Accordingly, spacers may be for instance be rigid or flexible, influencing either lipophilicity or hydrophilicity of the overall conjugate, and the like.

The spacer may comprise a linear or branched, optionally substituted C₁-C₂₀ hydrocarbyl, e.g. comprising up to 5 heteroatoms, more preferably C₁-C₁₂ hydrocarbyl, even more preferably C₂-C₆ hydrocarbyl, even more C₂-C₄ hydrocarbyl. The hydrocarbyl may preferably comprise at least one, optionally up to 4 or 5 heteroatoms preferably selected from N. It contains preferably one or two, more preferably one C—N bond.

Preferably, the spacer may be —[CHR⁶]ᵤ—NR⁷—, wherein R⁶ and R⁷ may each be independently selected from H and branched, unbranched or cyclic C₁-C₁₂ hydrocarbyl and wherein u may be an integer selected from 1, 2, 3, 4, 5, 6, 7, 8, 9 or 10. More preferably, R⁶ and R⁷ may be H, and u may be an integer selected from 2, 3 or 4, more preferably 2 or 4. Most preferably, R⁶ and R⁷ may be H and u may be 2 or 4. The spacer may preferably be —[CH₂]₂—NH— or —[CH₂]₄—NH—.

Accordingly, the spacer of the inventive conjugates may comprise or consist of Formula (2)(a) or (2)(a)′ or (2)(a)″:

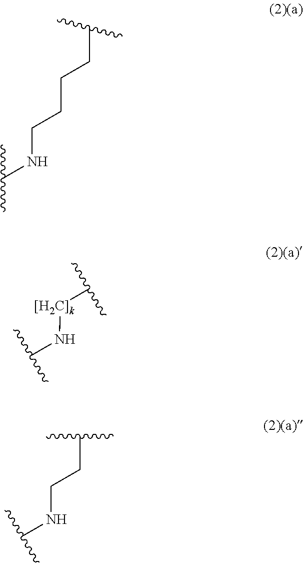

Formula (2)(a) is also referred to herein as “lysine spacer” or “Lys spacer”, as it reflects a lysine side chain spacer. For Formula (2)(a)′ k is an integer from 0 to 8, preferably 2 to 4.

Exemplified conjugates according to the invention (e.g. Ibu-PSMA, Ibu-Dα-PSMA, Ibu-Dβ-PSMA, Ibu-N-PSMA and Ibu-DAB-PSMA evaluated in the appended examples) comprise ibuprofen connected to the “branching point” via a spacer comprising or consisting of Formula (2)(a).

Accordingly, the spacer may comprise at least one amino acid residue or at least one side chain of an amino acid residue. As used herein, the term “amino acid residue” refers to a specific amino acid monomer as a moiety within the spacer.

An “amino acid” is any organic molecule comprising both an acidic (typically carboxy (—COOH)) and an amine (—NH₂) functional group. One or both of said groups may optionally be derivatized. The amino and the acidic group may be in any position relative to each other, but amino acids typically comprise 2-amino carboxylic acids, 3-amino carboxylic acids, 4-amino carboxylic acids, etc. The amine group may be attached to the 1^{st}, 2^{nd}, 3^{rd}, 4^{th}, 5^{th}, 6^{th}, 7^{th}, 8^{th}, 9^{th}, 10^{th} (etc.) up to the 20^{th} carbon atom of the amino acid(s). In other words, the amino acid(s) may be (an) alpha-, beta-, gamma-, delta-, epsilon- (etc.) up to an omega-amino acid(s). Preferably, the acidic group is a carboxy (—COOH) group. However, other acidic groups selected from —OPO₃H, —PO₃H, —OSO₃H or —SO₃H are also conceivable.

The amino acid may be a proteinogenic or a non-proteinogenic amino acid.

Proteinogenic amino acids are those twenty-two amino acids which are naturally incorporated into polypeptides. Except for selenocysteine and pyrrolysine, all proteinogenic amino acids (i.e., the twenty remaining proteinogenic amino acids) are encoded by the universal genetic code. The twenty-two proteinogenic amino acids are: arginine, histidine, lysine, aspartic acid, glutamic acid, serine, threonine, asparagine, glutamine, cysteine, glycine, proline, alanine, valine, isoleucine, leucine, methionine, phenylalanine, tyrosine, tryptophan, selenocysteine and pyrrolysine.

However, any organic compound with an amine (—NH₂) and a carboxylic acid (—COOH) functional group is an amino acid. In view thereof, any amino acid other than the twenty-two proteinogenic amino acids is referred to as “non-proteinogenic” amino acids. For example, non-proteinogenic amino acids may not be found in proteins (for example carnitine, GABA, levothyroxine, 2-aminoisobutyric acid and the neurotransmitter gamma-aminobutyric acid) or may not be produced directly and in isolation by standard cellular machinery (for example, hydroxyproline and selenomethionine). Non-proteinogenic amino acids may, for example, occur as intermediates in the metabolic pathways for standard amino acids—for example, ornithine and citrulline occur in the urea cycle. Examples include carnitine, GABA, levothyroxine, 2-aminoisobutyric acid, gamma-aminobutyric acid, hydroxyproline, selenomethionine, ornithine, citrulline, diaminobutyric acid, δ-Aminolevulinic acid, aminoisobutyric acid, diaminopimelic acid, cystathionine, lanthionine and Djenkolic acid. In the context of the present invention, for example diaminobutyric acid (DAB) is a particularly preferred non-proteinogenic amino acid.

The amino acid residue(s) may be derived from naturally occurring amino acid(s), or derivatives thereof. In particular, the amino acid residues(s) may be derived from alpha (α-) amino acid(s). The amino acid(s) may be (a) D- or L-amino acid(s).

For example, the amino acid(s) may be the D- or the L-enantiomer of an amino acid selected from the group arginine, asparagine, aspartate, cysteine, glutamate, glutamine, glycine, histidine, leucine, lysine, methionine, phenylalanine, proline, serine, threonine, tryptophan, tyrosine and/or valine.

Preferably, the amino acid is selected from lysine, aspartate, asparagine, diaminobutyric acid, phenylalanine, tyrosine, threonine, serine, proline, leucine, isoleucine, valine, arginine, histidine, glutamate, glutamine, and alanine. For example, the amino acid(s) may be the D- or the L-enantiomer of an amino acid selected from lysine, aspartate, asparagine, diaminobutyric acid, phenylalanine, tyrosine, threonine, serine, proline, leucine, isoleucine, valine, arginine, histidine, glutamate, glutamine, and alanine. For example, the amino acid(s) is/are (D-/L-) aspartate, glutamate or lysine, such as D-aspartate, D-glutamate or L-Lysine. For example, the amino acid(s) is/are (D-/L-) aspartate, asparagine, lysine or diaminobutyric acid.

For example, the further amino acid residue may be aspartate, asparagine or diaminobutyric acid.

The spacer may comprise 1, 2, 3, 4 or 5 amino acid residue(s), such as one or more D-aspartate, one or more D-glutamate and/or one or more L-Lysine residue. In conjugates comprising the D-enantiomer, the use of the D-enantiomer may provide the beneficial effect of further reducing the rate of metabolisation and thus clearance from the bloodstream. Preferably, the spacer may comprise 1 to 3 (preferably 1 or 2) of such amino acid residues, such as D-aspartate or D-glutamate residues or a (L-)lysine residue in combination with another amino acid residue (e.g., aspartate, asparagine or diaminobutyric acid). In other words, the spacer may comprise a peptide, which preferably consists of 1 to 5 amino acids, more preferably of 1 to 3 amino acids, even more preferably of 1 or 2 amino acids.

Accordingly, the inventive conjugates may comprise a spacer of Formula (2)(b):

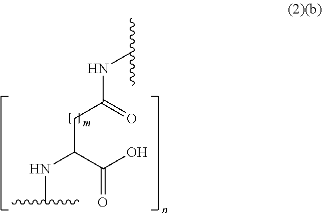

wherein
m is an integer selected from 1 or 2, and
n is an integer selected from 1, 2, 3, 4 or 5, preferably from 2 or 3.

Alternatively, the spacer may comprise an amino acid residue connected to the “branching point” via a linear or branched, optionally substituted, C₁-C₂₀ hydrocarbyl group comprising at least one N heteroatom.

Accordingly, the inventive conjugates may comprise a spacer of Formula (2)(c) or Formula (2)(c)′:

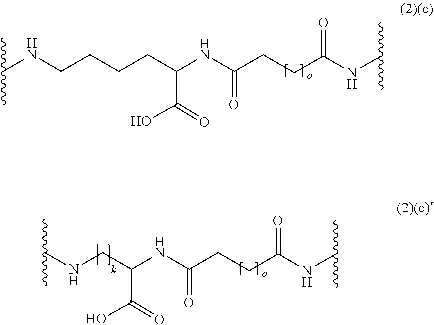

wherein o is an integer selected from 0, 1, 2, 3, 4, 5, 6, 7, 8, 9 or 10. Preferably, o may be 5.

For Formula (2)(c)′ k is an integer selected from 0 to 8, preferably 2, 3 or 4.

As described above, the spacer may comprise or consist of a (L-)lysine residue (e.g., as shown in Formula (2)(a)). In this context, the spacer may additionally comprise a further amino acid residue. In particular, the spacer may comprise or consist of Formula (2)(d) or (2)(d) or (2)(d)″:

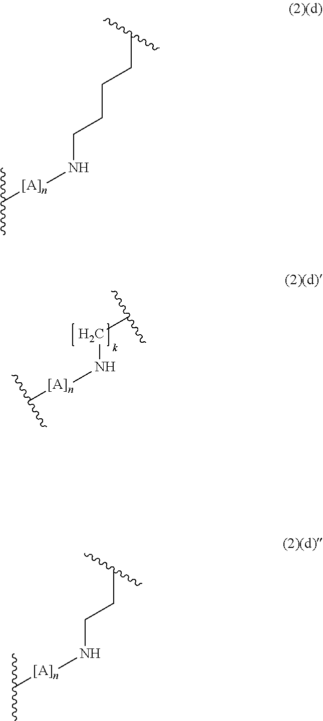

wherein A is an amino acid residue and n is an integer selected from 0, 1, 2, 3, 4, or 5, preferably from 0 or 1 and wherein k is an integer selected from 0 to 8, preferably 2 to 4.

In Formula (2)(d), A may be any amino acid residue as described above, in particular regarding the various preferred amino acids. For example, the further amino acid residue may be aspartate, asparagine or diaminobutyric acid.

For example, the spacer may comprise or consist of Formula (2)(d)(i) or Formula (2)(d)(i)′:

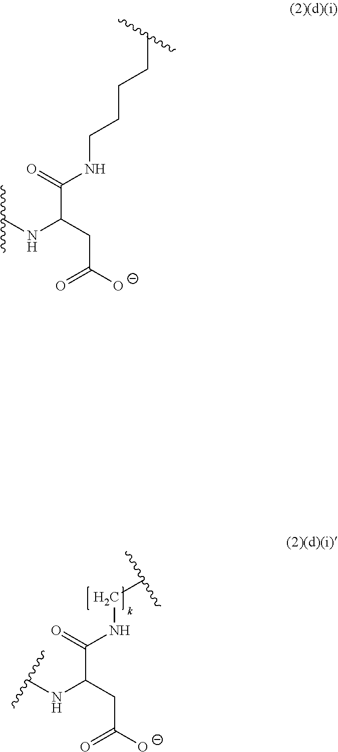

and wherein k is an integer selected from 0 to 8, preferably 2 to 4.

For example, the spacer may comprise or consist of Formula (2)(d)(ii) or Formula (2)(d)(ii)′:

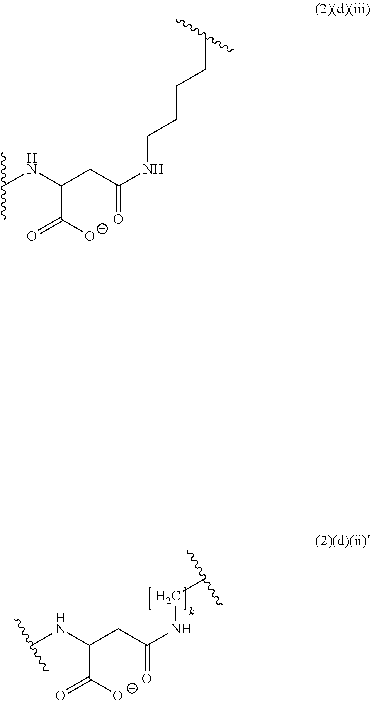

and wherein k is an integer selected from 0 to 8, preferably 2 to 4.

For example, the spacer may comprise or consist of Formula (2)(d)(iii) or Formula (2)(d)(iii)′:

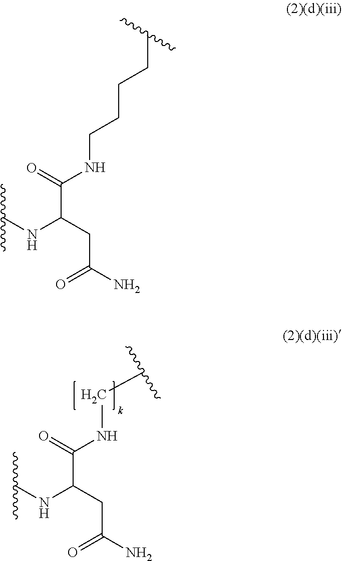

and wherein k is an integer selected from 0 to 8, preferably 2 to 4.

For example, the spacer may comprise or consist of Formula (2)(d)(iv) or Formula (2)(d)(iv)′:

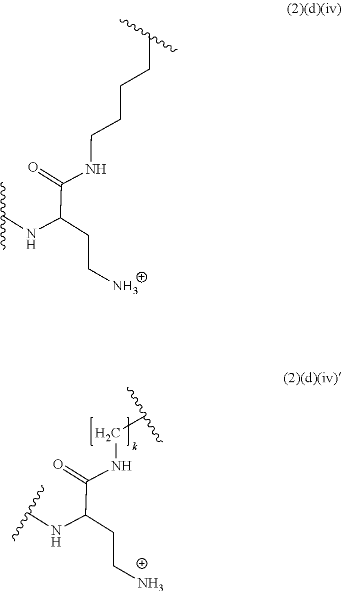

and wherein k is an integer selected from 0 to 8, preferably 2 to 4.

In view of the above, the present invention also provides a compound characterized by General Formula (1)(n):

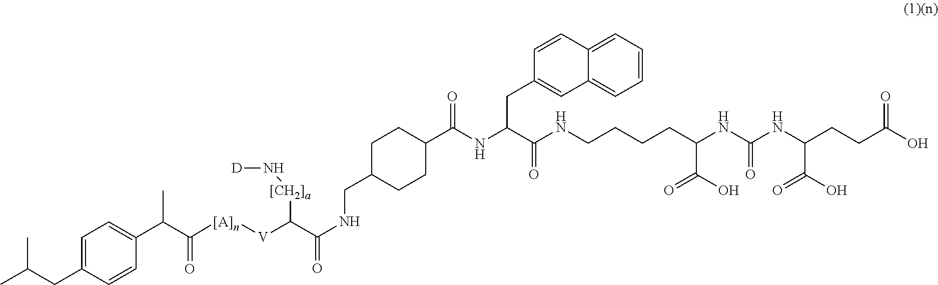

wherein D is a chelator (e.g., as described herein);
   A is an amino acid residue (e.g., as described herein) or an amino acid residue side chain thereof;
   V is selected from a single bond, N or NH, or an optionally substituted C₁-C₁₂ hydrocarbyl comprising up to 3 heteroatoms, wherein said heteroatom is preferably selected from N;
   a is an integer selected from 0, 1, 2, 3, 4, 5, 6, 7, 8, 9, or 10 (e.g., as described herein); and
   n is an integer selected from 0, 1, 2, 3, 4, or 5, preferably from 0 or 1;
      or a pharmaceutically acceptable salt, ester, solvate or radiolabeled complex thereof.

Accordingly, the present invention also provides a compound characterized by General Formula (1)(o):

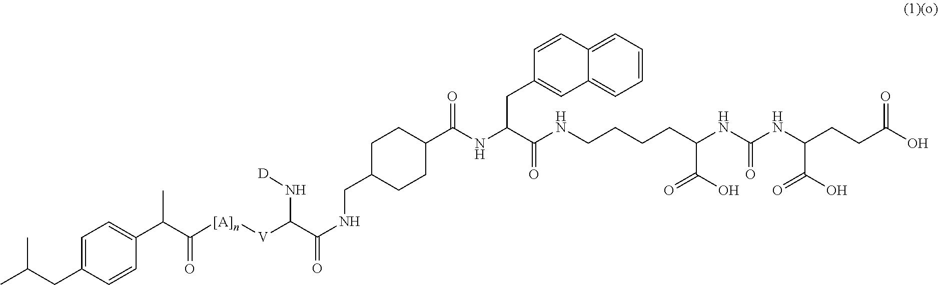

wherein D is a chelator (e.g., as described herein);
   A is an amino acid residue (e.g., as described herein) or an amino acid residue side chain thereof;
   V is selected from a single bond, N or NH, or an optionally substituted C₁-C₁₂ hydrocarbyl comprising up to 3 heteroatoms, wherein said heteroatom is preferably selected from N;
   a is an integer selected from 0, 1, 2, 3, 4, 5, 6, 7, 8, 9, or 10 (e.g., as described herein); and
   n is an integer selected from 0, 1, 2, 3, 4, or 5, preferably from 0 or 1;
   k is an integer selected from 0, 1, 2, 3, 4, or 5, 6, 7 or 8, preferably from 2, 3, or 4,
      or a pharmaceutically acceptable salt, ester, solvate or radiolabeled complex thereof.

V in formula (1)(n) or 1(o) may contain 1 or 2 C—N-bond(s), preferably 1 C—N bond.

V may represent an NH group in both Formula (1)(n) or (1)(o).

In particular, the present invention also provides a compound characterized by Formula (6)(a) or Formula (6)(a)′:

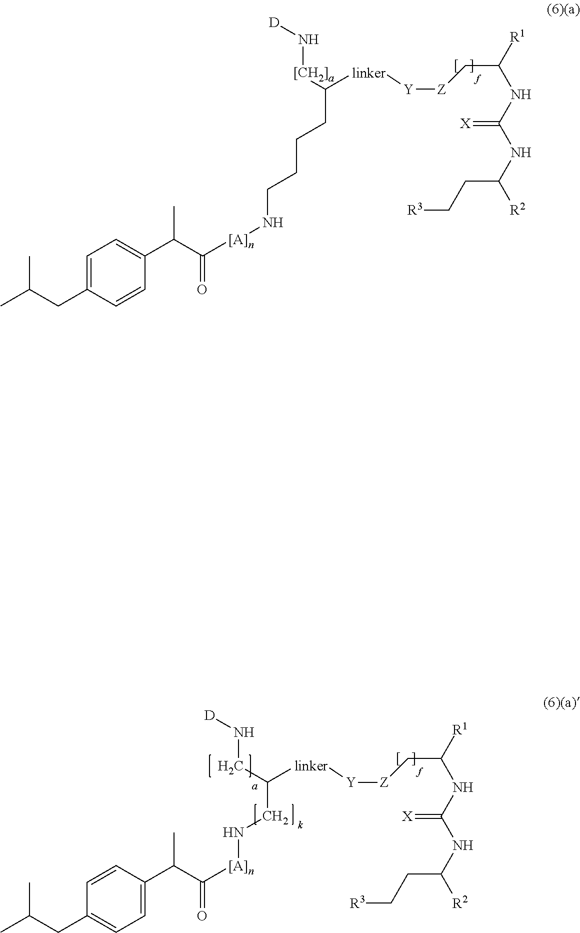

wherein D, the linker and a are as defined herein for General Formula (1)(a) (and, preferably, its embodiments);
   X, Y, Z, R¹, R², R³ and f are as defined herein for General Formula (3) (and, preferably, its embodiments);
   A is an amino acid residue (e.g., as described herein);
   n is an integer selected from 0, 1, 2, 3, 4, or 5, preferably from 0 or 1; and
   k is an integer selected from 0, 1, 2, 3, 4, 5, 6, 7 or 8, preferably from 2, 3, or 4,
      or a pharmaceutically acceptable salt, ester, solvate or radiolabeled complex thereof.

In particular, the present invention also provides a compound characterized by Formula (6)(b) or (6)(b)′:

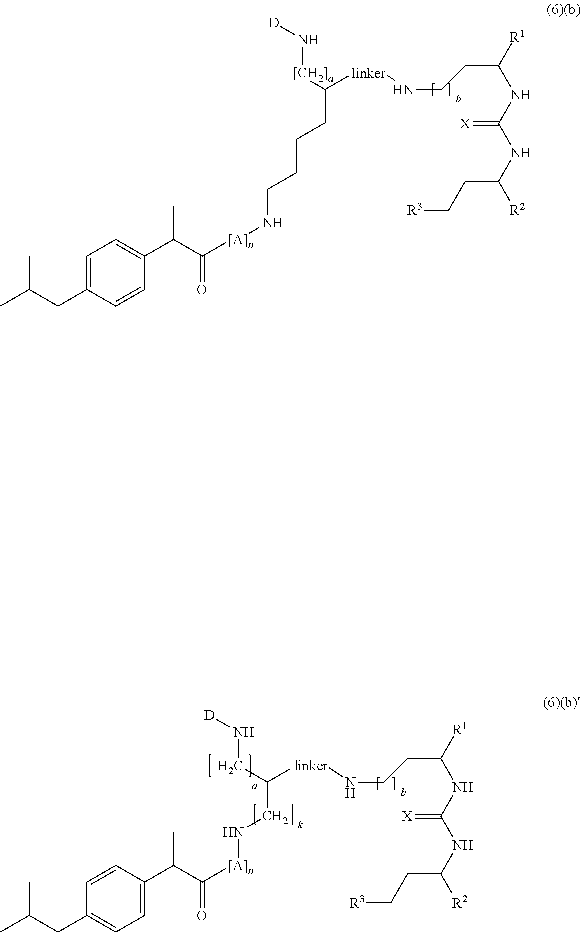

wherein D, the linker and a are as defined herein for General Formula (1)(a) (and, preferably, its embodiments);
   X, R¹, R², R³ and b are as defined herein for General Formula (3)(ii) (and, preferably, its embodiments);
   A is an amino acid residue (e.g., as described herein);
   n is an integer selected from 0, 1, 2, 3, 4, or 5, preferably from 0 or 1; and
   k is an integer selected from 0, 1, 2, 3, 4, 5, 6, 7 or 8, preferably from 2, 3, or 4,
      or a pharmaceutically acceptable salt, ester, solvate or radiolabeled complex thereof.

In particular, the present invention also provides a compound characterized by Formula (6)(c) or (6)(c)′:

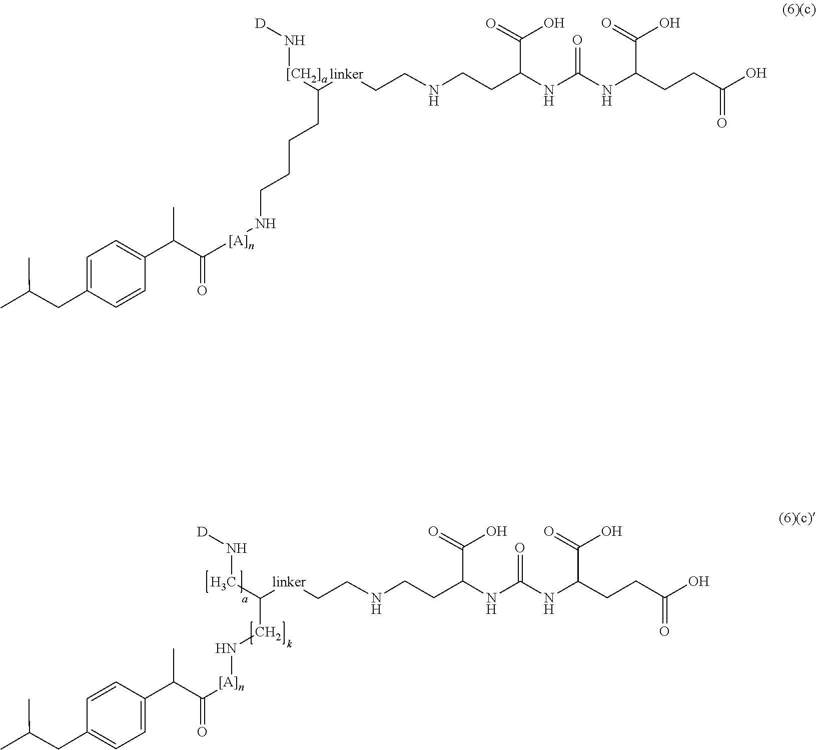

wherein D, the linker and a are as defined herein for General Formula (1)(a) (and, preferably, its embodiments);
   A is an amino acid residue (e.g., as described herein);
   n is an integer selected from 0, 1, 2, 3, 4, or 5, preferably from 0 or 1; and
   k is an integer selected from 0, 1, 2, 3, 4, 5, 6, 7 or 8, preferably from 2, 3, or 4,
      or a pharmaceutically acceptable salt, ester, solvate or radiolabeled complex thereof.

In particular, the present invention also provides a compound characterized by Formula (6)(d) or (6)(d)′:

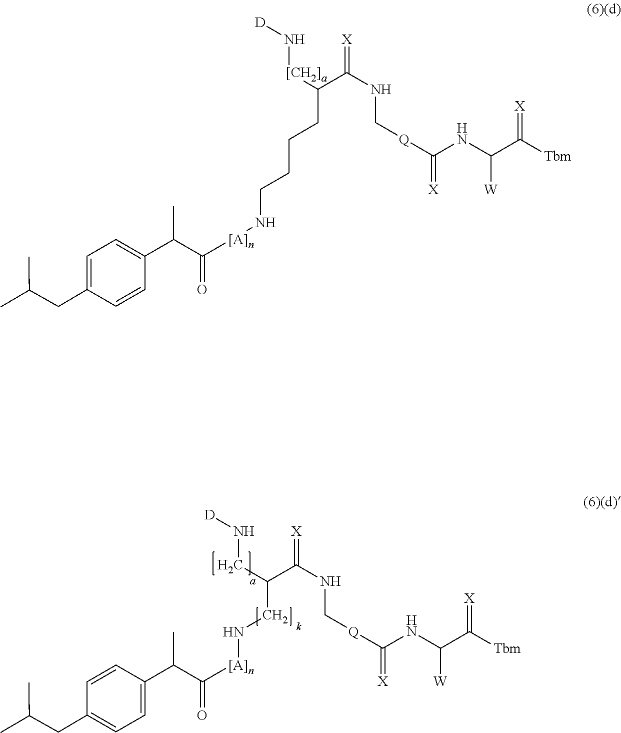

wherein D, Tbm and a are as defined herein for General Formula (1)(a) (and, preferably, its embodiments);
   X, Q and W are as defined herein for General Formula (4) (and, preferably, its embodiments);
   A is an amino acid residue (e.g., as described herein);
   n is an integer selected from 0, 1, 2, 3, 4, or 5, preferably from 0 or 1; and
   k is an integer selected from 0, 1, 2, 3, 4, 5, 6, 7 or 8, preferably from 2, 3, or 4,
      or a pharmaceutically acceptable salt, ester, solvate or radiolabeled complex thereof.

In particular, the present invention also provides a compound characterized by Formula (6)(e) or (6)(e)′:

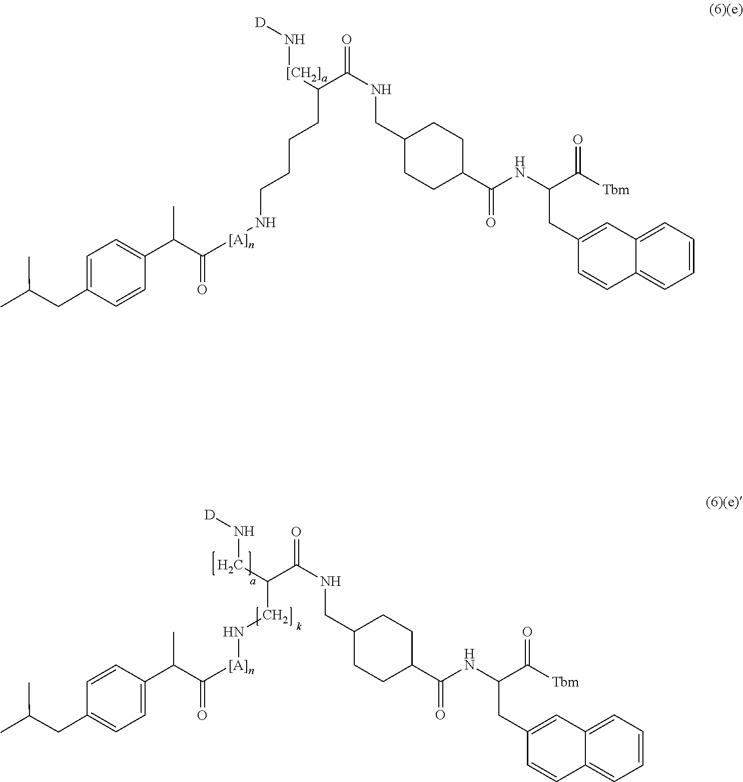

wherein D, Tbm and a are as defined herein for General Formula (1)(a) (and, preferably, its embodiments);
   A is an amino acid residue (e.g., as described herein);
   n is an integer selected from 0, 1, 2, 3, 4, or 5, preferably from 0 or 1; and
   k is an integer selected from 0, 1, 2, 3, 4, 5, 6, 7 or 8, preferably from 2, 3, or 4,
      or a pharmaceutically acceptable salt, ester, solvate or radiolabeled complex thereof.

In particular, the present invention also provides a compound characterized by Formula (6)(f) or (6)(f)′:

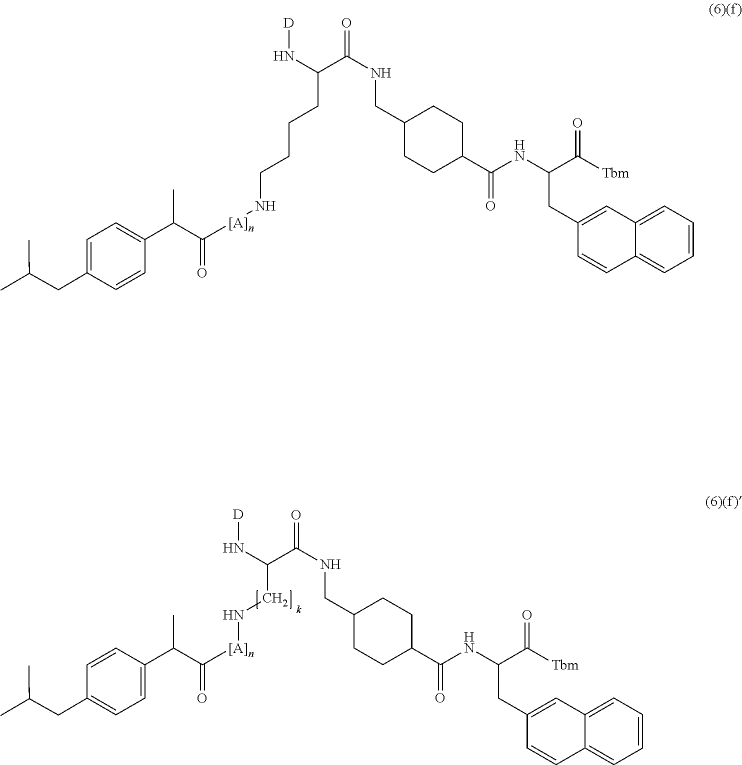

wherein D and Tbm are as defined herein for General Formula (1)(a) (and, preferably, its embodiments);
   A is an amino acid residue (e.g., as described herein);
   n is an integer selected from 0, 1, 2, 3, 4, or 5, preferably from 0 or 1; and
   k is an integer selected from 0, 1, 2, 3, 4, 5, 6, 7 or 8, preferably from 2, 3, or 4,
      or a pharmaceutically acceptable salt, ester, solvate or radiolabeled complex thereof.

In particular, the present invention also provides a compound characterized by Formula (6)(g) or (6)(g)′:

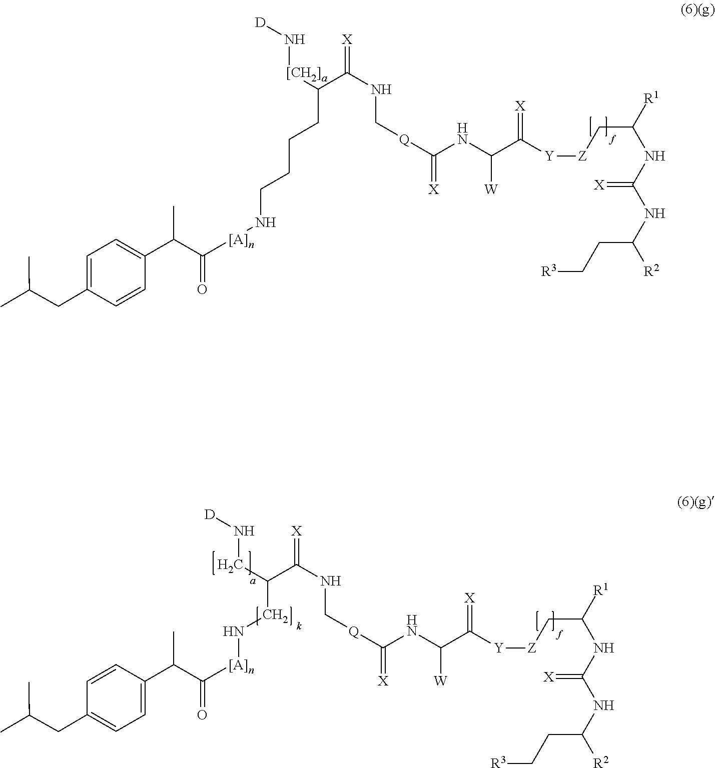

wherein D and a are as defined herein for General Formula (1)(a) (and, preferably, its embodiments);
   Y, Z, R¹, R², R³ and f are as defined herein for General Formula (3) (and, preferably, its embodiments);
   X, Q and W are as defined herein for General Formula (4) (and, preferably, its embodiments);
   A is an amino acid residue (e.g., as described herein);
   n is an integer selected from 0, 1, 2, 3, 4, or 5, preferably from 0 or 1; and
   k is an integer selected from 0, 1, 2, 3, 4, 5, 6, 7 or 8, preferably from 2, 3, or 4,
      or a pharmaceutically acceptable salt, ester, solvate or radiolabeled complex thereof.

In particular, the present invention also provides a compound characterized by Formula (6)(h) or (6)(h)′:

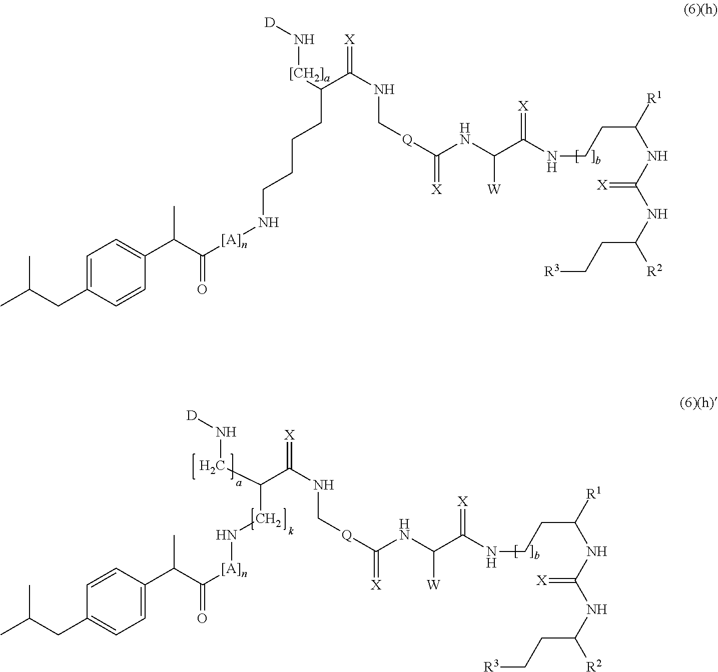

wherein D and a are as defined herein for General Formula (1)(a) (and, preferably, its embodiments);
   R¹, R², R³ and b are as defined herein for General Formula (3)(ii) (and, preferably, its embodiments);
   X, Q and W are as defined herein for General Formula (4) (and, preferably, its embodiments);
   A is an amino acid residue (e.g., as described herein);
   n is an integer selected from 0, 1, 2, 3, 4, or 5, preferably from 0 or 1; and
   k is an integer selected from 0, 1, 2, 3, 4, 5, 6, 7 or 8, preferably from 2, 3, or 4,
      or a pharmaceutically acceptable salt, ester, solvate or radiolabeled complex thereof.

In particular, the present invention also provides a Compound characterized by Formula (6)(i) or (6)(i)′:

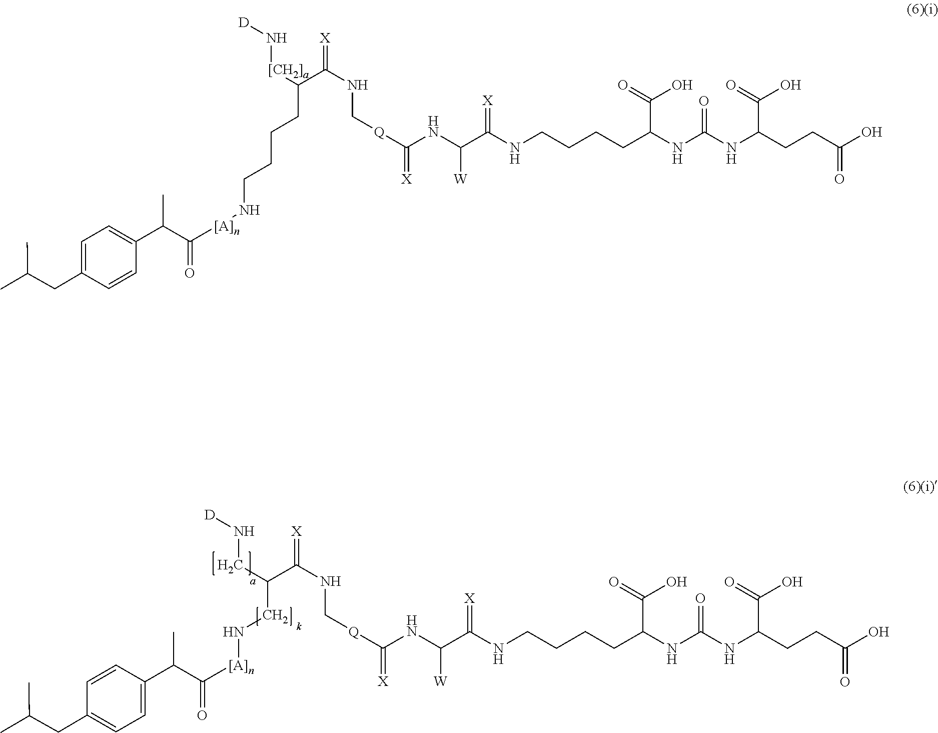

wherein D and a are as defined herein for General Formula (1)(a) (and, preferably, its embodiments);
   X, Q and W are as defined herein for General Formula (4) (and, preferably, its embodiments);
   A is an amino acid residue (e.g., as described herein);
   n is an integer selected from 0, 1, 2, 3, 4, or 5, preferably from 0 or 1; and
   k is an integer selected from 0, 1, 2, 3, 4, 5, 6, 7 or 8, preferably from 2, 3, or 4,
      or a pharmaceutically acceptable salt, ester, solvate or radiolabeled complex thereof.

In particular, the present invention also provides a compound characterized by Formula (6)(j) or (6)(i)′:

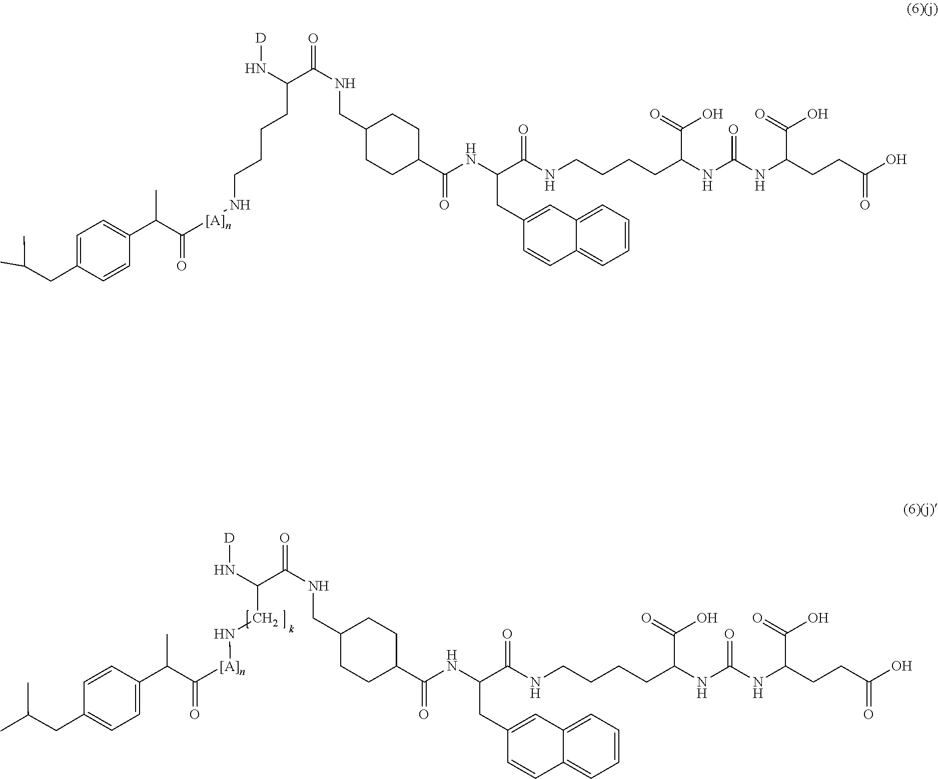

wherein D is a chelator as described herein;
   X, Q and W are as defined herein for General Formula (4) (and, preferably, its embodiments);
   A is an amino acid residue (e.g., as described herein);
   n is an integer selected from 0, 1, 2, 3, 4, or 5, preferably from 0 or 1; and
   k is an integer selected from 0, 1, 2, 3, 4, 5, 6, 7 or 8, preferably from 2, 3, or 4,
      or a pharmaceutically acceptable salt, ester, solvate or radiolabeled complex thereof.

The most preferred amino acid residues in the context of Formulas (6)(a)-(6)(j) are aspartate, asparagine and diaminobutyric acid, or, alternatively, -[A]ₙ is absent.

For example, the present invention also provides a compound characterized by Formula (7)(a) or (7)(a)′:

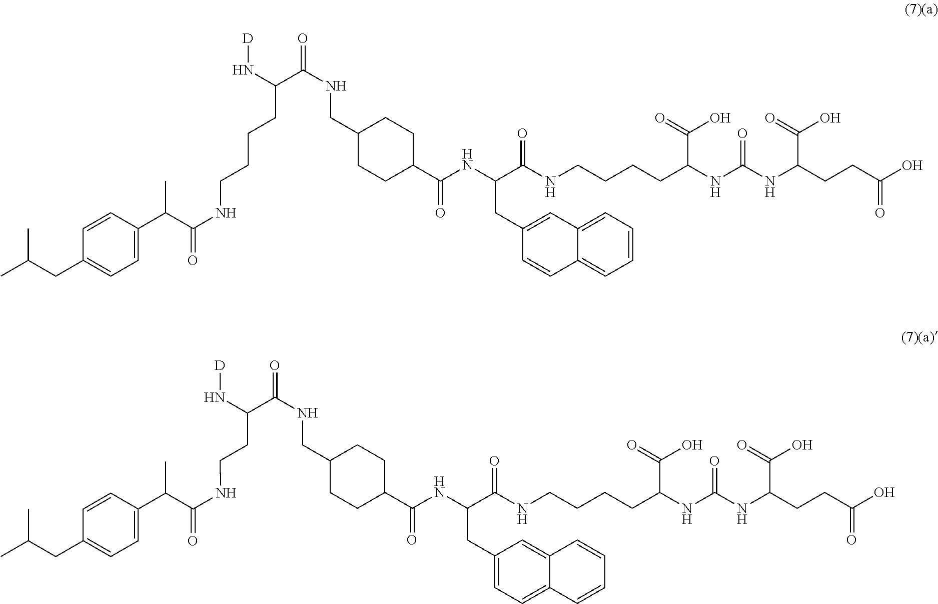

wherein D is a chelator as described herein;
   or a pharmaceutically acceptable salt, ester, solvate or radiolabeled complex thereof.

For example, the present invention also provides a compound characterized by Formula (7)(b) or (7)(b)′:

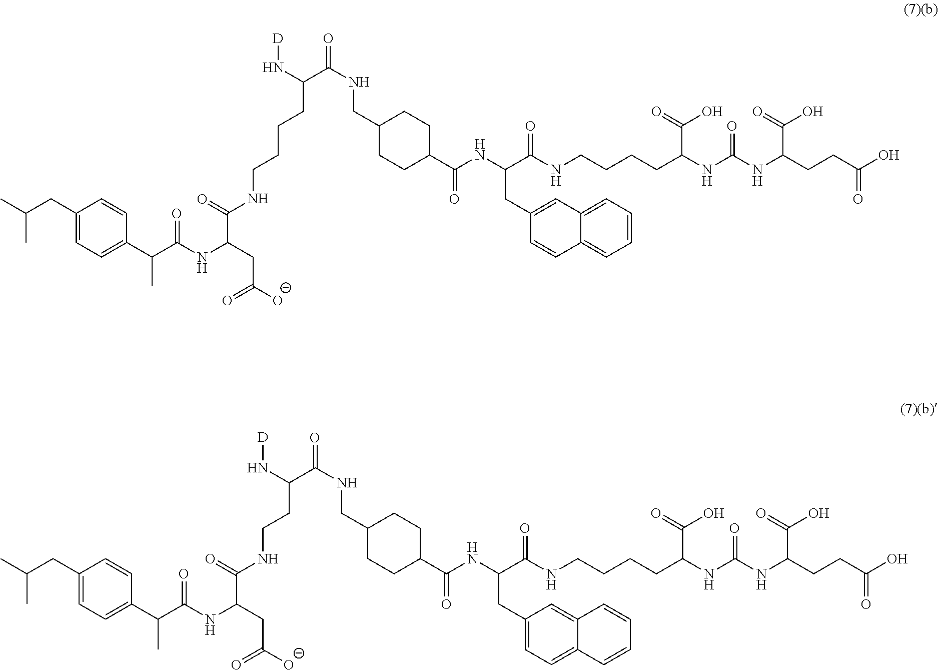

wherein D is a chelator as described herein;
   or a pharmaceutically acceptable salt, ester, solvate or radiolabeled complex thereof.

For example, the present invention also provides a compound characterized by Formula (7)(c) or (7)(c)′:

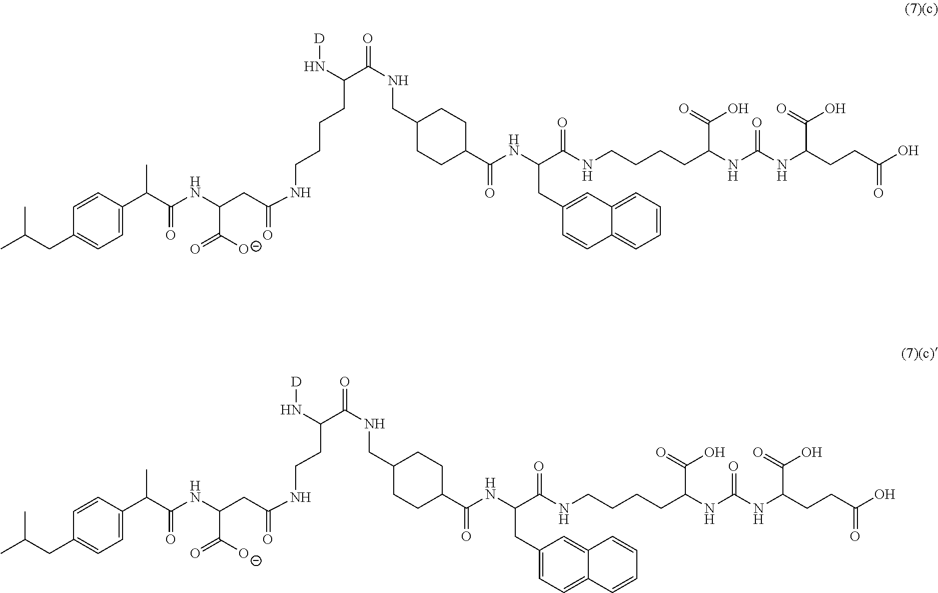

wherein D is a chelator as described herein;
   or a pharmaceutically acceptable salt, ester, solvate or radiolabeled complex thereof.

For example, the present invention also provides a compound characterized by Formula (7)(d) or (7)(d)′:

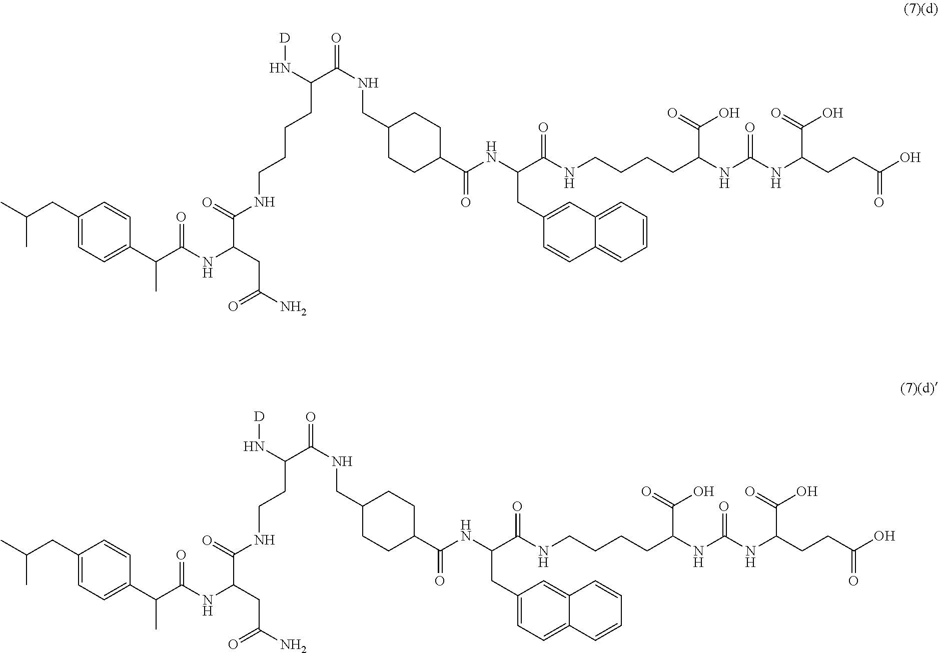

wherein D is a chelator as described herein;
   or a pharmaceutically acceptable salt, ester, solvate or radiolabeled complex thereof.

For example, the present invention also provides a compound characterized by Formula (7)(e) or (7)(e)′:

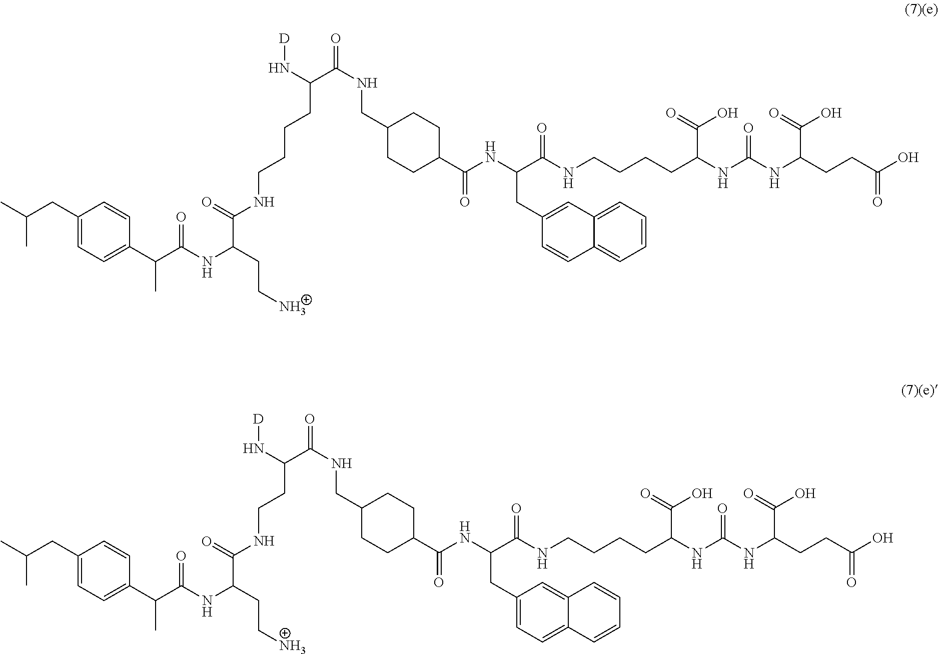

wherein D is a chelator as described herein;
   or a pharmaceutically acceptable salt, ester, solvate or radiolabeled complex thereof.

For all of above Formulae (7)(a), (7)(a)′, (7)(b), (7)(b)′, (7)(c), (7)(c)′, (7)(d), (7)(d)′, (7)(e) and (7)(e)′ the lysine side chain as the spacer or as a portion of the spacer consists of 2 or 4 methylene groups linking the branching point via the lysine side chain NH group to the ibuprofen group. Alternatively, 0, 1, 3, 5, 6, 7 or 8 methylene groups may be employed for any of the compounds of these Formulae.

### Chelator

The inventive conjugates may further comprise a chelator. For example, a chelator may be useful for coordination of a radiometal, for example to provide a radiolabeled conjugate (also referred to as “radioligand”).

The terms “chelator” or “chelating moiety” are used herein interchangeably to refer to polydentate (multiple bonded) ligands capable of forming two or more separate coordinate bonds with (“coordinating”) a central (metal) ion. Specifically, such molecules or molecules sharing one electron pair may also be referred to as “Lewis bases”. The central (metal) ion is usually coordinated by two or more electron pairs to the chelating agent. The terms, “bidentate chelating agent”, “tridentate chelating agent”, and “tetradentate chelating agent” are art-recognized and refer to chelating agents having, respectively, two, three, and four electron pairs readily available for simultaneous donation to a metal ion coordinated by the chelating agent. Usually, the electron pairs of a chelating agent forms coordinate bonds with a single central (metal) ion; however, in certain examples, a chelating agent may form coordinate bonds with more than one metal ion, with a variety of binding modes being possible.

The terms “coordinating” and “coordination” refer to an interaction in which one multi-electron pair donor coordinatively bonds (is “coordinated”) to, i.e. shares two or more unshared pairs of electrons with, one central (metal) ion.

The chelating agent is preferably chosen based on its ability to coordinate the desired central (metal) ion, such as a radionuclide as described herein.

Accordingly, the chelator D may be characterized by one of the following Formulas (5a)-(5jj):

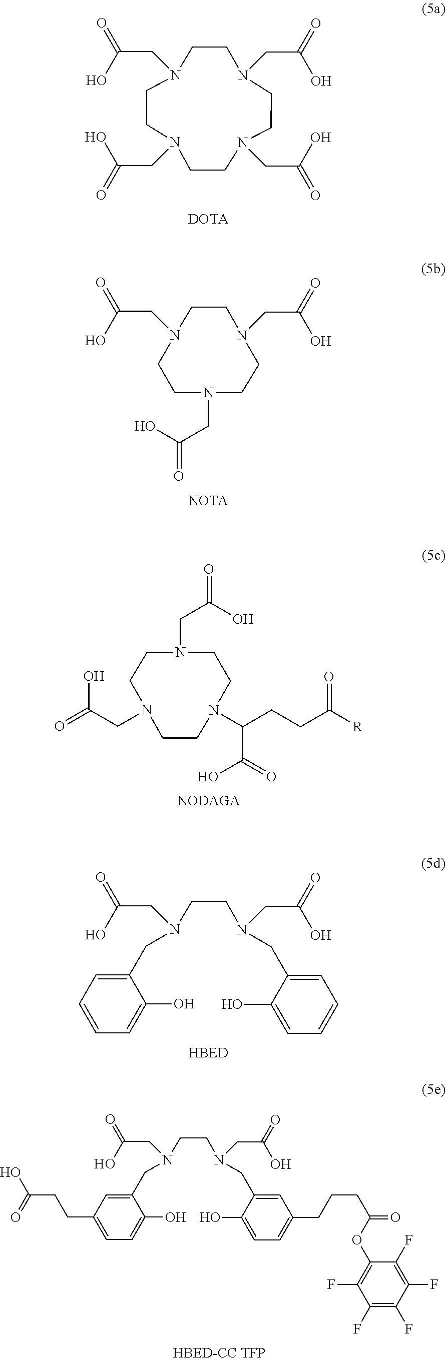

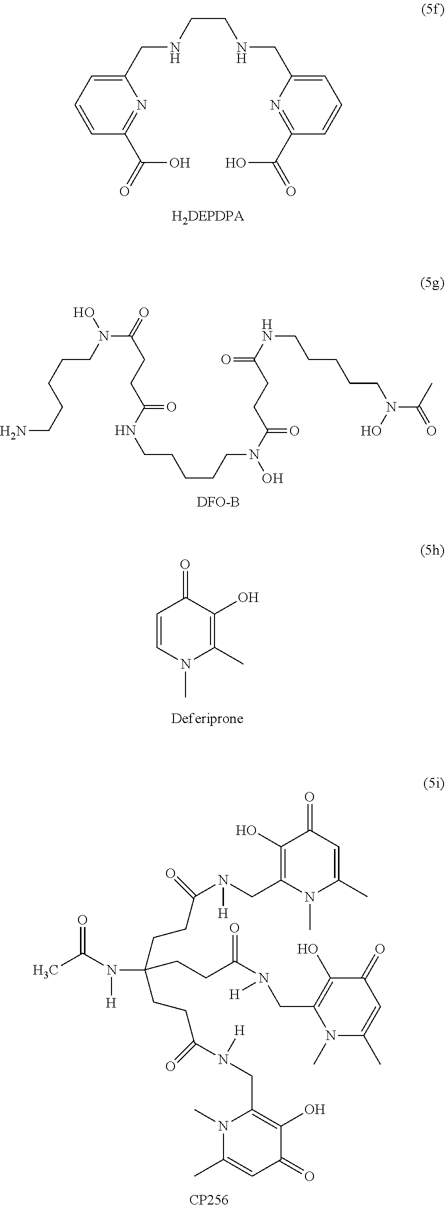

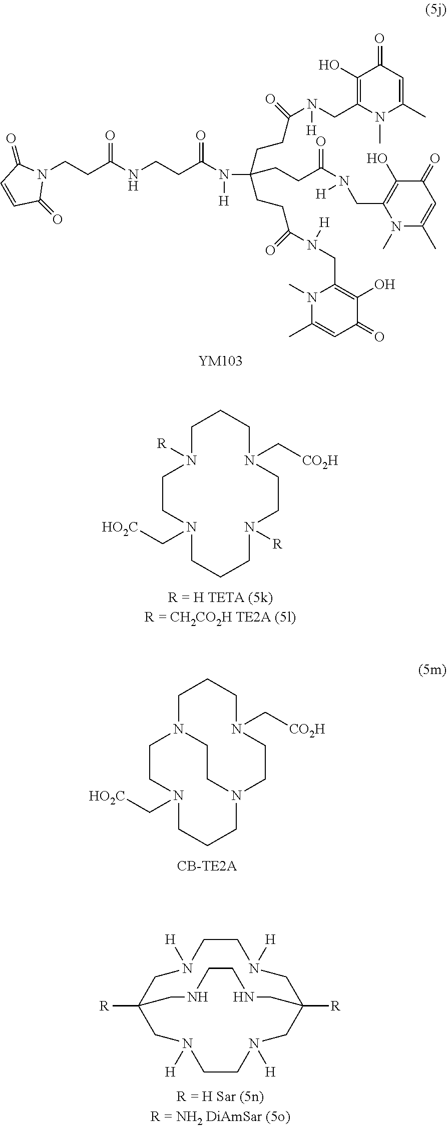

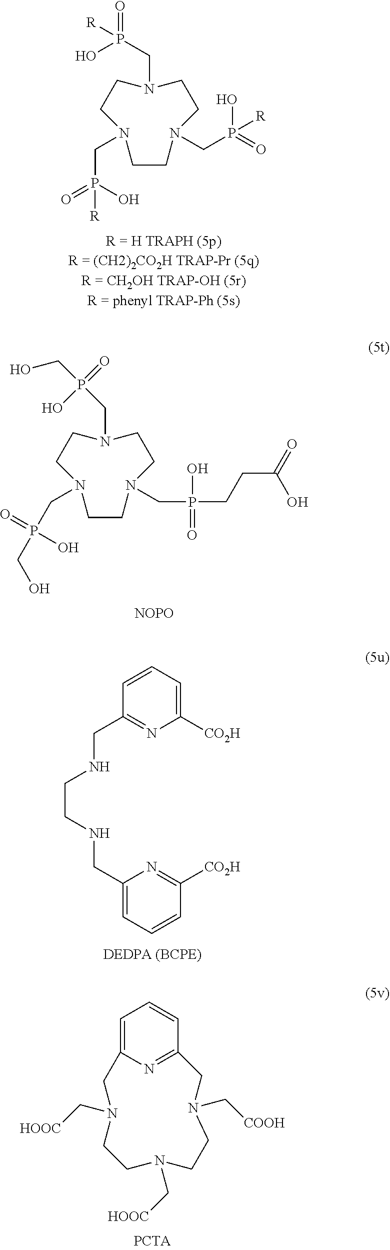

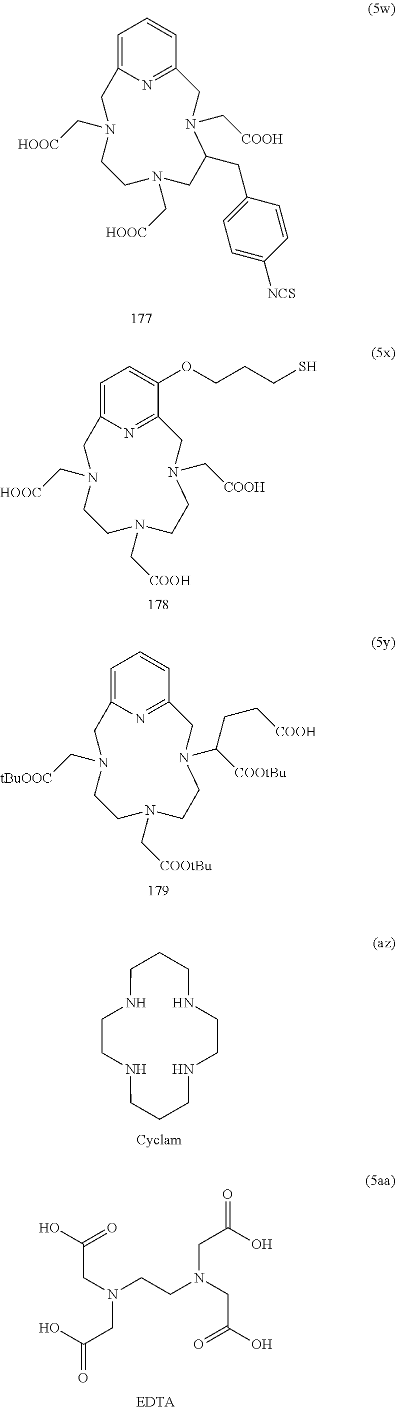

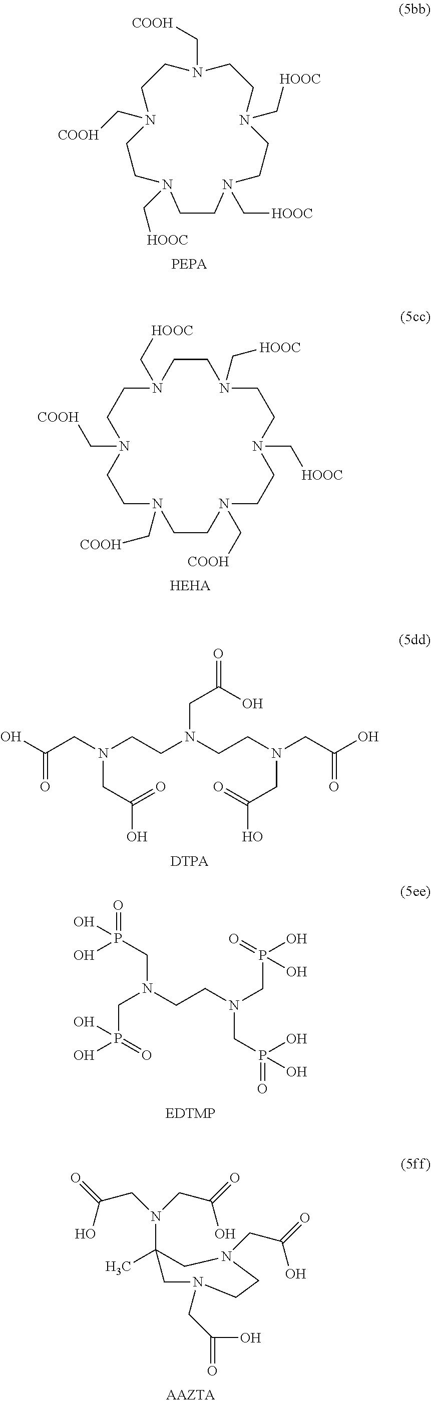 [SMILES: O=C(O)CN1CCN(CC(=O)O)CCN(CC(=O)O)CCN(CC(=O)O)CCN(CC(=O)O)CC1 ; O=C(O)CN1CCN(CC(=O)O)CCN(CC(=O)O)CCN(CC(=O)O)CCN(CC(=O)O)CCN(CC(=O)O)CC1 ; O=C(O)CN(CCN(CC(=O)O)CC(=O)O)CCN(CC(=O)O)CC(=O)O ; O=P(O)(O)CN(CCN(CP(=O)(O)O)CP(=O)(O)O)CP(=O)(O)O ; CC1(N(CC(=O)O)CC(=O)O)CN(CC(=O)O)CCN(CC(=O)O)C1]

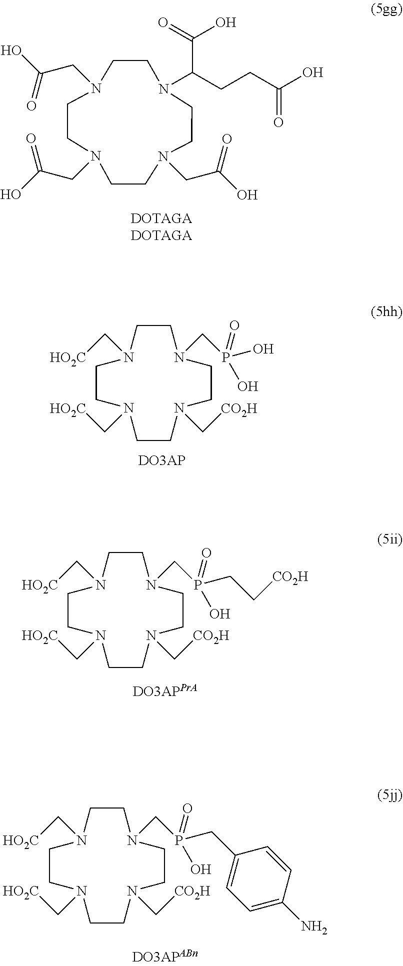 [SMILES: O=C(O)CCC(C(=O)O)N1CCN(CC(=O)O)CCN(CC(=O)O)CCN(CC(=O)O)CC1 ; O=C(O)CN1CCN(CC(=O)O)CCN(CP(=O)(O)O)CCN(CC(=O)O)CC1 ; O=C(O)CCP(=O)(O)CN1CCN(CC(=O)O)CCN(CC(=O)O)CCN(CC(=O)O)CC1 ; Nc1ccc(CP(=O)(O)CN2CCN(CC(=O)O)CCN(CC(=O)O)CCN(CC(=O)O)CC2)cc1]

The chelator (D) may be selected from any one of the chelators (5a)-(5jj) as described above.

Preferably, the chelator (D) is selected from 1,4,7,10-tetraazacyclododecane-1,4,7,10-tetraacetic acid (DOTA), N,N″-bis[2-hydroxy-5-(carboxyethyl)-benzyl]ethylenediamine-N,N″-diacetic acid (HBED-CC), 1,4,7-triazacyclononane-1,4,7-triacetic acid (NOTA), 2-(4,7-bis(carboxymethyl)-1,4,7-triazonan-1-yl)pentanedioic acid (NODAGA), [2-(4,7,10-tris(carboxymethyl)-1,4,7,10-tetraazacyclododecan-1-yl)-pentanedioic acid (DOTAGA), 1,4,7-triazacyclononane phosphinic acid (TRAP), 1,4,7-triazacydononane-1-[methyl(2-carboxyethyl)-phosphinic acid]-4,7-bis[methyl(2-hydroxymethyl)phosphinic acid] (NOPO), 3,6,9, 15-tetraazabicyclo[9,3,1]pentadeca-1(15),11,13-triene-3,6,9-triacetic acid (PCTA), N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide (DFO), and Diethylenetriaminepentaacetic acid (DTPA), or derivatives thereof.

More preferably, the chelator may be DOTA (1,4,7,10-tetraazacyclododecane-1,4,7,10-tetraacetic acid, which may be characterized by Formula (5a)), NODAGA (2-(4,7-bis(carboxymethyl)-1,4,7-triazonan-1-yl)-pentanedioic acid, which may be characterized by Formula (5c)), or derivatives thereof. In some embodiments, the chelator may be NODAGA.

For example, the chelator may be DOTA. Advantageously, DOTA effectively forms complexes with diagnostic (e.g. ⁶⁸Ga) and therapeutic (e.g. ⁹⁰Y or ¹⁷⁷Lu) radionuclides and thus enables the use of the same conjugate for both imaging and therapeutic purposes, i.e. as a theragnostic agent. DOTA derivatives capable of complexing Scandium radionuclides (⁴³Sc, ⁴⁴Sc, ⁴⁷Sc), including DO3AP (which may be characterized by Formula (5hh)), DO3AP^{PrA} (which may be characterized by Formula (5ii)), or DO3AP^{ABn} (which may be characterized by Formula (5jj)) may also be preferred and are described in Kerdjoudj et al. Dalton Trans., 2016, 45, 1398-1409.

Other preferred chelators in the context of the present invention include N,N″-bis[2-hydroxy-5-(carboxyethyl)benzyl]ethylenediamine-N,N″-diacetic acid (HBED-CC), 1,4,7-triazacyclo-nonane-1,4,7-triacetic acid (NOTA), 2-(4,7,10-tris(carboxymethyl)-1,4,7,10-tetra-azacyclododecan-1-yl)-pentanedioic acid (DOTAGA), 1,4,7-triazacyclononane phosphinic acid (TRAP), 1,4,7-triazacydo-nonane-1-[methyl(2-carboxyethyl)-phosphinic acid]-4,7-bis-[methyl(2-hydroxymethyl)-phosphinic acid] (NOPO),3,6,9,15-tetra-azabicyclo[9,3,1]-pentadeca-1(15),11,13-triene-3,6,9-triacetic acid (PCTA), N′-{5-[Acetyl(hydroxy)amino]-pentyl}-N-[5-({4-[(5-aminopentyl)(hydroxy)amino]-4-oxobutanoyl}-amino)pentyl]-N-hydroxysuccinamide (DFO), and Diethylene-triaminepentaacetic acid (DTPA).

The chelator group, for example the DOTA group, may be complexed with a central (metal) ion, in particular a radionuclide as defined herein. Alternatively, the chelator group, for example DOTA, may not be complexed with a central (metal) ion, in particular a radionuclide as defined herein, and may thus be present in uncomplexed form. In cases where the chelator (e.g. DOTA) is not complexed with said metal ion, the carboxylic acid groups of the chelator can be in the form of a free acid, or in the form of a salt.

In the following, specific exemplified conjugates according to the present invention are described, which are particularly preferred:

A preferred exemplified conjugate according to the present invention is shown in Formula (8)(a) or (8)(a)′:

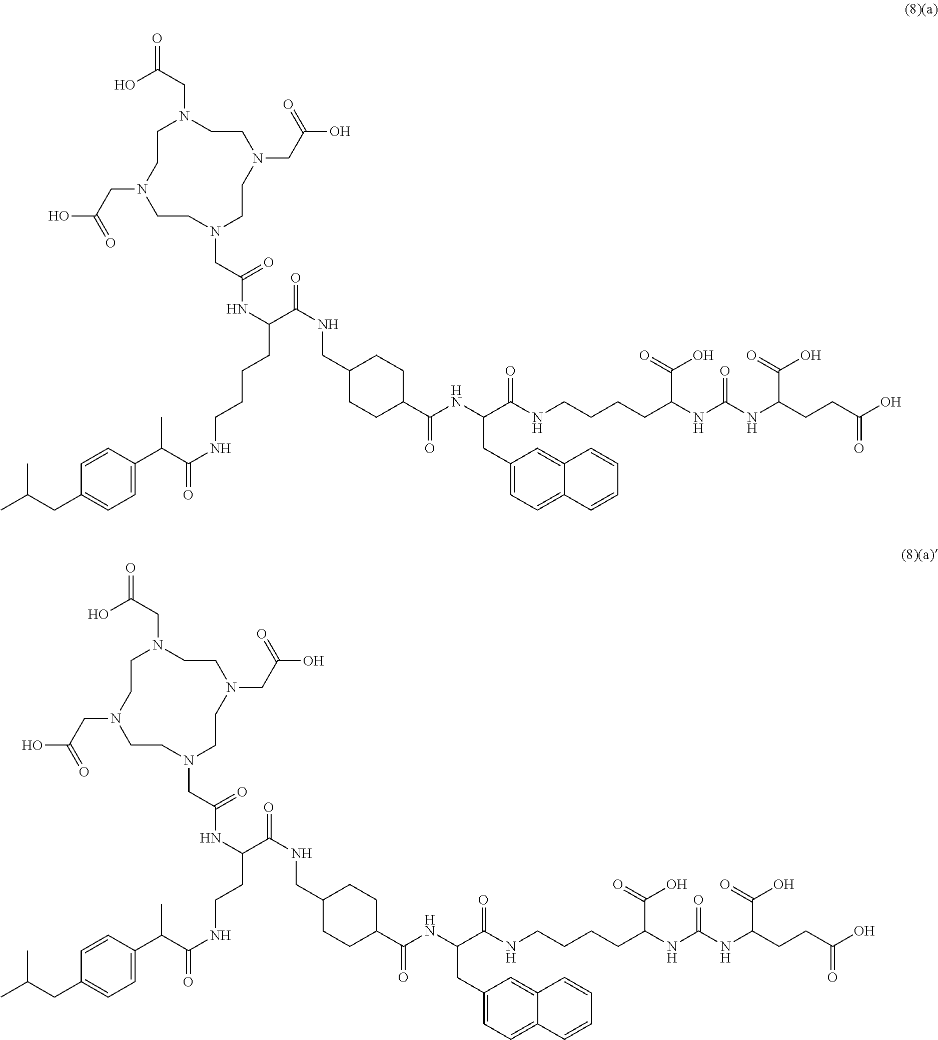 [SMILES: CC(C)Cc1ccc(C(C)C(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)cc1 ; CC(C)Cc1ccc(C(C)C(=O)NCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)cc1]

(Formula (8)(a) also referred to as “Ibu-PSMA”) or a pharmalogically acceptable salt, ester, solvate or radiolabeled complex thereof.

Another preferred exemplified conjugate according to the present invention is shown in Formula (8)(b) or (8)(b)′:

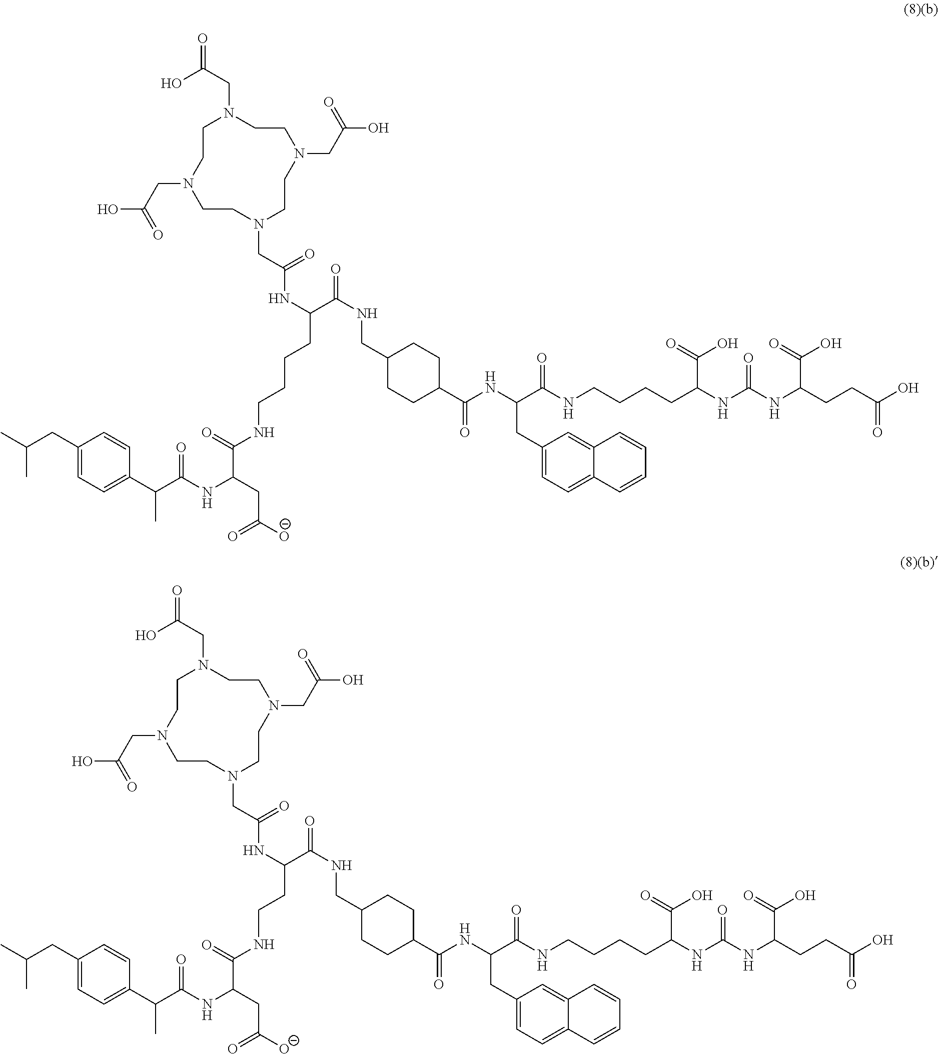 [SMILES: CC(C)Cc1ccc(C(C)C(=O)NC(CC(=O)[O-])C(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)cc1 ; CC(C)Cc1ccc(C(C)C(=O)NC(CC(=O)[O-])C(=O)NCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)cc1]

(Formula (8)(b) is also referred to as “Ibu-Dα-PSMA”) or a pharmalogically acceptable salt, ester, solvate or radiolabeled complex thereof.

Another preferred exemplified conjugate according to the present invention is shown in Formula (8)(c) or (8)(c)′:

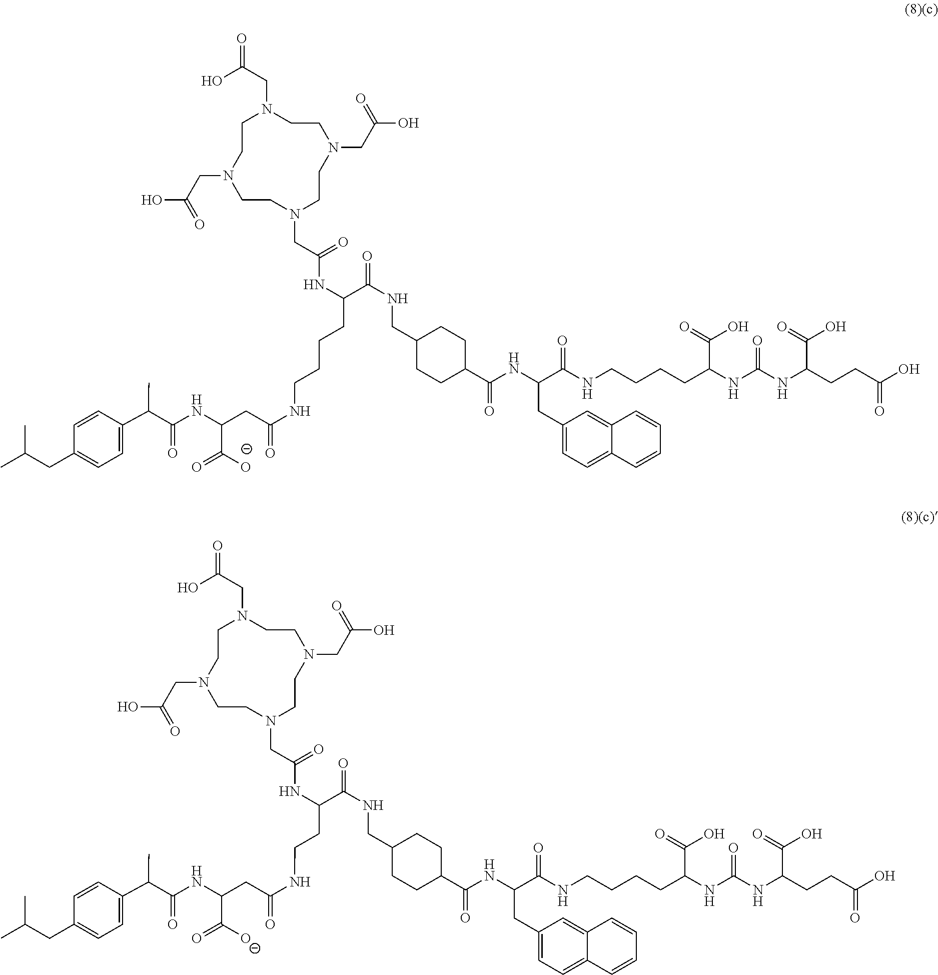 [SMILES: CC(C)Cc1ccc(C(C)C(=O)NC(CC(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)C(=O)[O-])cc1 ; CC(C)Cc1ccc(C(C)C(=O)NC(CC(=O)NCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)C(=O)[O-])cc1]

(Formula (8)(c) is also referred to as “Ibu-Dβ-PSMA”) or a pharmalogically acceptable salt, ester, solvate or radiolabeled complex thereof.

Another preferred exemplified conjugate according to the present invention is shown in Formula (8)(d) or Formula (8)(d)′:

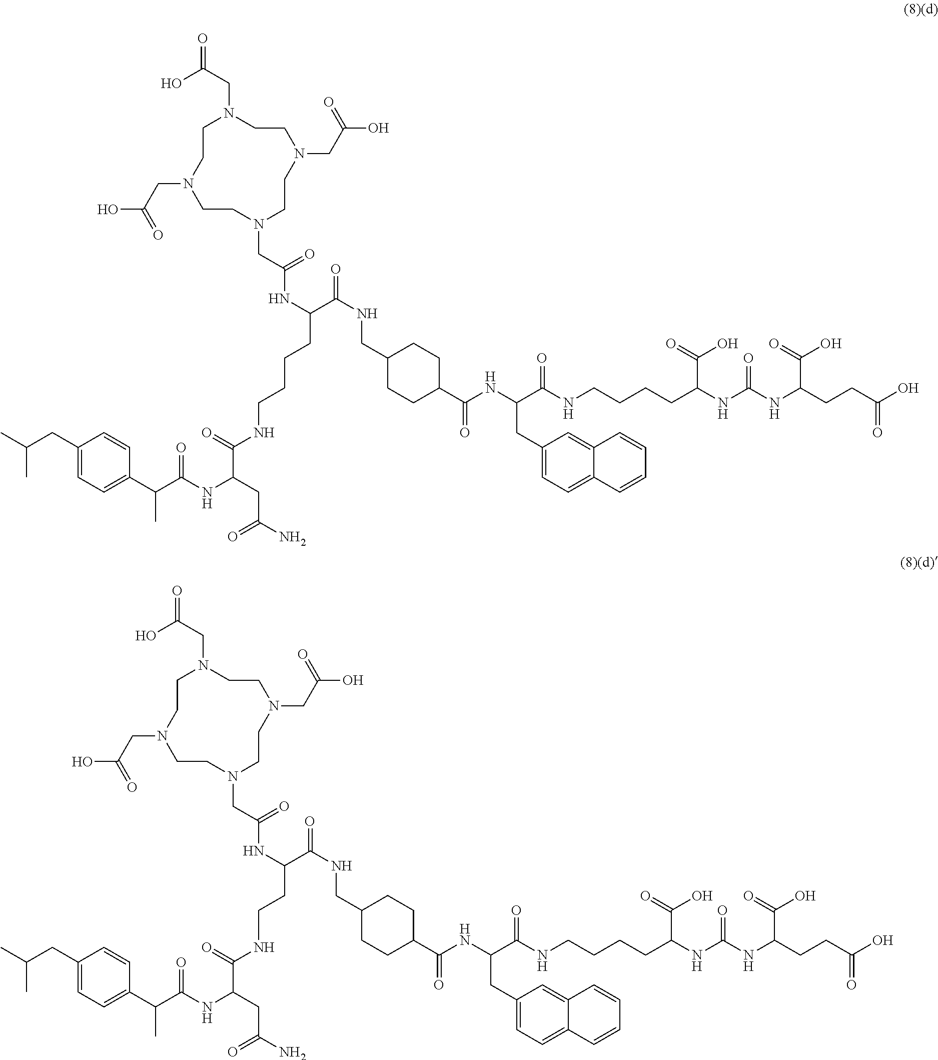 [SMILES: CC(C)Cc1ccc(C(C)C(=O)NC(CC(N)=O)C(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)cc1 ; CC(C)Cc1ccc(C(C)C(=O)NC(CC(N)=O)C(=O)NCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)cc1]

(Formula (8)(d) is also referred to as “Ibu-N-PSMA”) or a pharmalogically acceptable salt, ester, solvate or radiolabeled complex thereof.

Another preferred exemplified conjugate according to the present invention is shown in Formula (8)(e) or (8)(e)′:

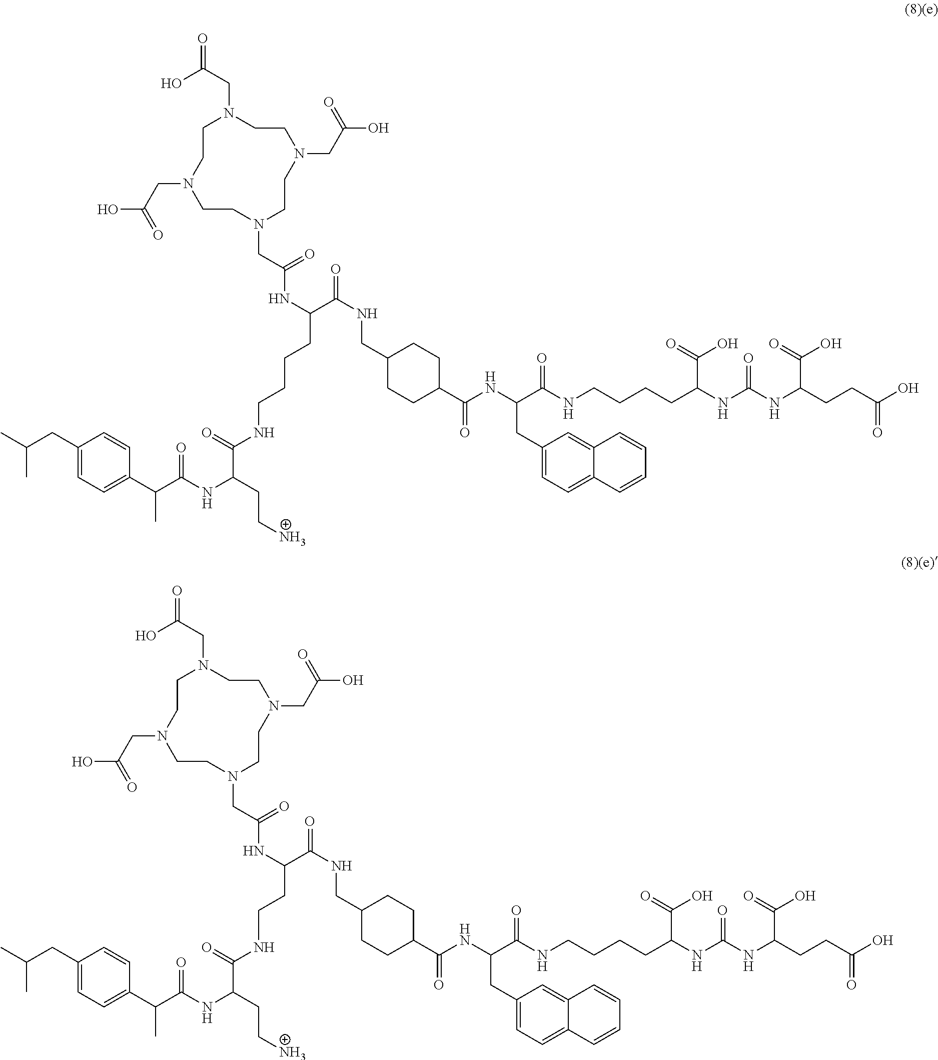 [SMILES: CC(C)Cc1ccc(C(C)C(=O)NC(CC[NH3+])C(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)cc1 ; CC(C)Cc1ccc(C(C)C(=O)NC(CC[NH3+])C(=O)NCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)cc1]

(Formula (8)(e) is also referred to as “Ibu-DAB-PSMA”) or a pharmalogically acceptable salt, ester, solvate or radiolabeled complex thereof.

All of Formula (8)(a), (8)(a)′, (8)(b), (8)(b)′, (8)(c), (8)(c)′, (8)(d), (8)(d)′, (8)(e) and (8)(e)′ are also disclosed to comprise 0, 1, 3, 5, 6, 7 or 8 —[CH]₂ moieties linking the lysine side chain NH group of the spacer with the branching point, instead of 2 and 4 methylene groups as defined by the above Formulae.

### Pharmaceutically Acceptable Salts

The present invention further encompasses pharmaceutically acceptable salts of the conjugates (compounds) described herein.

The preparation of pharmaceutical compositions is well known to the person skilled in the art. Pharmaceutically acceptable salts of the conjugates of the invention can be prepared by conventional procedures, such as by reacting any free base and/or acid of a conjugate according to the invention with at least a stoichiometric amount of the desired salt-forming acid or base, respectively.

Pharmaceutically acceptable salts of the inventive include salts with inorganic cations such as sodium, potassium, calcium, magnesium, zinc, and ammonium, and salts with organic bases. Suitable organic bases include N-methyl-D-glucamine, argmme, benzathine, diolamine, olamine, procame and tromethamine. Pharmaceutically acceptable salts according to the invention also include salts derived from organic or inorganic acids. Suitable anions include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulfate, napsylate, nitrate, oleate, pamoate, phosphate, polygalacturonate, stearate, succinate, sulfate, sulfosalicylate, tannate, tartrate, terephthalate, tosylate and triethiodide.

### Complexed/Non-Complexed Forms

The present invention further encompasses the conjugates (compounds) described herein, wherein the chelator (D) may be complexed with a metal ion (such as a radionuclide) or may not be complexed.

The term “radionuclide” (or “radioisotope”) refers to isotopes of natural or artificial origin with an unstable neutron to proton ratio that disintegrates with the emission of corpuscular (i.e. protons (alpha-radiation) or electrons (beta-radiation) or electromagnetic radiation (gamma-radiation). In other words, radionuclides undergo radioactive decay. The chelator (D) may be complexed with any known radionuclide. Said radionuclide which may preferably be useful for cancer imaging or therapy. Such radionuclides include, without limitation, ⁹⁴Tc, ^{99m}Tc, ⁹⁰In, ¹¹¹In, ⁶⁷Ga, ⁶⁸Ga, ⁸⁶Y, ⁹⁰Y, ¹⁷⁷Lu, ¹⁵¹Tb, ¹⁸⁶Re, ¹⁸⁸Re, ⁶¹Cu, ⁶⁷Cu, ⁵⁵Co, ⁵⁷Co, ⁴³Sc, ⁴⁴Sc, ⁴⁷Sc, ²²⁵Ac, ²¹³Bi, ²¹²Bi, ²¹²Pb, ²²⁷Th, ¹⁵³Sm, ¹⁶⁶Ho, ¹³²Gd, ¹⁵³Gd, ¹⁵⁷Gd, or ¹⁶⁶Dy. The choice of suitable radionuclides may depend inter alia on the chemical structure and chelating capability of the chelator (D), and the intended application of the resulting (complexed) conjugate (e.g. diagnostic vs. therapeutic). On the other hand, the chelator (D) may be selected in view of the envisaged radionuclide/radiometal. For instance, the beta-emitters such as ⁹⁰Y, ¹³¹I, ¹⁶¹Tb and ¹⁷⁷Lu may be used for concurrent systemic radionuclide therapy. Providing DOTA as a chelator may advantageously enable the use of either ⁶⁸Ga, ^{43,44,47}Sc, ¹⁷⁷Lu, ¹⁶¹Tb, ²²⁵Ac, ²¹³Bi, ²¹²Bi, ²¹²Pb as radionuclides.

In some preferred embodiments, the radionuclide may be ¹⁷⁷Lu. In some preferred embodiments, the radionuclide may be ⁴⁴Sc. In some preferred embodiments, the radionuclide may be ⁶⁴Cu. In some preferred embodiments, the radionuclide may be ⁶⁸Ga. Most preferably, the radionuclide is ¹⁷⁷Lu.

It is within the skill and knowledge of the skilled person in the art to select suitable combinations conjugates (compounds) and radionuclides. For instance, in some preferred embodiments, the chelator may be DOTA and the radionuclide may be ¹⁷⁷Lu. In other preferred embodiments, the chelator may be DOTA and the radionuclide may be ⁶⁸Ga. In other preferred embodiments, the chelator may be DOTA and the radionuclide may be ⁴⁴Sc. In yet further preferred embodiments, the chelator may be DOTA and the radionuclide may be ⁶⁴Cu. In other preferred embodiments, the chelator may be NODAGA and the radionuclide may be ⁶⁴Cu.

### Esters and Prodrugs

The present invention further encompasses the inventive conjugates (compounds) in their esterified form, in particular where free carboxylic acid groups are esterified. Such esterified compounds may be prodrug forms of the inventive conjugates. Suitable ester prodrugs include various alkyl esters, including saturated and unsaturated C₈-C₁₈ fatty acids.

### Enantiomers

The conjugates (compounds) disclosed herein may exist in particular geometric or stereoisomeric forms. In addition, compounds may also be optically active. The inventive conjugates may also include cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. If, for instance, a particular enantiomer of a group or conjugate is desired, it may be prepared by asymmetric synthesis, or by derivation with a chiral auxiliary, where the resulting diastereomeric mixture is separated and the auxiliary group cleaved to provide the pure desired enantiomers. Alternatively, where the group or conjugate contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

A “stereoisomer” is one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereo isomer of the compound and less than about 20% by weight of other stereo isomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereo isomer of the compound and less than about 3% by weight of the other stereoisomers of the compound.

Accordingly, all Formulas disclosed herein comprise enantiomers and/or stereoisomers thereof.

### Radiolabeled Complexes

According to a further aspect, the present invention relates to the use of the inventive conjugate (compound) for the preparation of radiolabeled complexes or to their use as a medicament or as a precursor of a medicament. Such radiolabeled complexes preferably comprise a conjugate (compound) according to the present invention, and a radionuclide. The chelator (D) preferably coordinates the radionuclide, forming a radiolabeled complex. Suitable radionuclides may be selected from theragnostic metal isotopes and comprise without limitation, ⁹⁴Tc, ^{99m}Tc, ⁹⁰In, ¹¹¹In, ⁶⁷Ga, ⁶⁸Ga, ⁸⁶Y, ⁹⁰Y, ¹⁷⁷Lu, ¹⁵¹Tb, ¹⁸⁶Re, ¹⁸⁸Re, ⁶⁴Cu, ⁶⁷Cu, ⁵⁵Co, ⁵⁷Co, ⁴³Sc, ⁴⁴Sc, ⁴⁷Sc, ²²⁵Ac, ²¹³Bi, ²¹²Bi, ²¹²Pb, ²²⁷Th, ⁵³Sn, ¹⁶⁶Ho, ¹⁵²Gd, ¹⁵³Gd, ¹⁵⁷Gd, or ¹⁶⁶Dy.

According to a further aspect, the present invention also provides a complex comprising a radionuclide (preferably as described herein) and a conjugate according to the invention.

### Pharmaceutical Compositions

According to a further aspect, the present invention also provides a pharmaceutical composition comprising the inventive conjugate (compound) (including pharmaceutically acceptable salts, esters, solvates or radiolabeled complexes as described herein), and a pharmaceutically acceptable carrier and/or excipient.

The term “pharmaceutically acceptable” refers to a compound or agent that is compatible with the inventive conjugate and does not interfere with and/or substantially reduce its diagnostic or therapeutic activities. Pharmaceutically acceptable carriers preferably have sufficiently high purity and sufficiently low toxicity to make them suitable for administration to a subject to be treated.

### Formulations, Carriers and Excipients

Pharmaceutically acceptable excipients can exhibit different functional roles and include, without limitation, diluents, fillers, bulking agents, carriers, disintegrants, binders, lubricants, glidants, coatings, solvents and co-solvents, buffering agents, preservatives, adjuvants, antioxidants, wetting agents, anti-foaming agents, thickening agents, sweetening agents, flavouring agents and humectants.

Suitable pharmaceutically acceptable excipients are typically chosen based on the formulation of the (pharmaceutical) composition.

For (pharmaceutical) compositions in liquid form, useful pharmaceutically acceptable excipients in general include solvents, diluents or carriers such as (pyrogen-free) water, (isotonic) saline solutions such phosphate or citrate buffered saline, fixed oils, vegetable oils, such as, for example, groundnut oil, cottonseed oil, sesame oil, olive oil, corn oil, ethanol, polyols (for example, glycerol, propylene glycol, polyetheylene glycol, and the like); lecithin; surfactants; preservatives such as benzyl alcohol, parabens, chlorobutanol, phenol, ascorbic acid, thimerosal, and the like; isotonic agents such as sugars, polyalcohols such as manitol, sorbitol, or sodium chloride; aluminum monostearate or gelatin; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. Buffers may be hypertonic, isotonic or hypotonic with reference to the specific reference medium, i.e. the buffer may have a higher, identical or lower salt content with reference to the specific reference medium, wherein preferably such concentrations of the aforementioned salts may be used, which do not lead to damage of cells due to osmosis or other concentration effects. Reference media are e.g. liquids occurring in in vivo methods, such as blood, lymph, cytosolic liquids, or other body liquids, or e.g. liquids, which may be used as reference media in in vitro methods, such as common buffers or liquids. Such common buffers or liquids are known to a skilled person.

Liquid (pharmaceutical) compositions administered via injection and in particular via i.v. injection should preferably be sterile and stable under the conditions of manufacture and storage. Such compositions are typically formulated as parenterally acceptable aqueous solutions that are pyrogen-free, have suitable pH, are isotonic and maintain stability of the active ingredient(s).

For liquid pharmaceutical compositions, suitable pharmaceutically acceptable excipients and carriers include water, typically pyrogen-free water; isotonic saline or buffered (aqueous) solutions, e.g phosphate, citrate etc. buffered solutions. Particularly for injection of the inventive (pharmaceutical) compositions, water or preferably a buffer, more preferably an aqueous buffer, may be used, which may contain a sodium salt, e.g. at least 50 mM of a sodium salt, a calcium salt, e.g. at least 0.01 mM of a calcium salt, and optionally a potassium salt, e.g. at least 3 mM of a potassium salt.

The sodium, calcium and, optionally, potassium salts may occur in the form of their halogenides, e.g. chlorides, iodides, or bromides, in the form of their hydroxides, carbonates, hydrogen carbonates, or sulfates, etc. Without being limited thereto, examples of sodium salts include e.g. NaCl, NaI, NaBr, Na₂CO₃, NaHCO₃, Na₂SO₄., examples of the optional potassium salts include e.g. KCl, Kl, KBr, K₂CO₃, KHCO₃, K₂SO₄, and examples of calcium salts include e.g. CaCl₂, CaI₂, CaBr₂, CaCO₃, CaSO₄, Ca(OH)₂. Furthermore, organic anions of the aforementioned cations may be contained in the buffer.

Buffers suitable for injection purposes as defined above, may contain salts selected from sodium chloride (NaCl), calcium chloride (CaCl₂) and optionally potassium chloride (KCl), wherein further anions may be present additional to the chlorides. CaCl₂ can also be replaced by another salt like KCl. Typically, the salts in the injection buffer are present in a concentration of at least 50 mM sodium chloride (NaCl), at least 3 mM potassium chloride (KCl) and at least 0.01 mM calcium chloride (CaCl₂). The injection buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of cells due to osmosis or other concentration effects.

For (pharmaceutical) compositions in (semi-)solid form, suitable pharmaceutically acceptable excipients and carriers include binders such as microcrystalline cellulose, gum tragacanth or gelatin; starch or lactose; sugars, such as, for example, lactose, glucose and sucrose; starches, such as, for example, corn starch or potato starch; cellulose and its derivatives, such as, for example, sodium carboxymethylcellulose, ethylcellulose, cellulose acetate; disintegrants such as alginic acid; lubricants such as magnesium stearate; glidants such as stearic acid, magnesium stearate; calcium sulphate, colloidal silicon dioxide and the like; sweetening agents such as sucrose or saccharin; and/or flavoring agents such as peppermint, methyl salicylate, or orange flavoring.

Generally, (pharmaceutical) compositions for topical administration can be formulated as creams, ointments, gels, pastes or powders. (Pharmaceutical) compositions for oral administration can be formulated as tablets, capsules, liquids, powders or in a sustained release format. However, according to preferred embodiments, the inventive (pharmaceutical) composition is administered parenterally, in particular via intravenous or intratumoral injection, and is accordingly formulated in liquid or lyophilized form for parenteral administration as discussed elsewhere herein. Parenteral formulations are typically stored in vials, IV bags, ampoules, cartridges, or prefilled syringes and can be administered as injections, inhalants, or aerosols, with injections being preferred.

The (pharmaceutical) composition may be provided in lyophilized form. Lyophilized (pharmaceutical) compositions are preferably reconstituted in a suitable buffer, advantageously based on an aqueous carrier, prior to administration.

The (pharmaceutical) composition preferably comprises a safe and effective amount of the inventive conjugate(s) or radiolabeled complexe(s).

As used herein, “safe and effective amount” means an amount of the agent(s) that is sufficient to allow for diagnosis and/or significantly induce a positive modification of the disease to be treated or prevented. At the same time, however, a “safe and effective amount” is small enough to avoid serious side-effects, that is to say to permit a sensible relationship between advantage and risk. A “safe and effective amount” will furthermore vary in connection with the particular condition to be diagnosed or treated and also with the age and physical condition of the patient to be treated, the severity of the condition, the duration of the treatment, the nature of the accompanying therapy, of the particular pharmaceutically acceptable excipient or carrier used, and similar factors.

The inventive conjugates (compounds) are also provided for use in the preparation of a medicament, preferably for the use in treating cancer or for treating cancer, in particular for treating and/or preventing prostate cancer, pancreatic cancer, renal cancer or bladder cancer.

### Kit

According to a further aspect, the present invention also provides a kit comprising the inventive conjugate(s) (including pharmaceutically acceptable salts, esters, solvates or radiolabeled complexes thereof) and/or the pharmaceutical composition(s) of the invention.

Optionally, the kit may comprise at least one further agent as defined herein in the context of the pharmaceutical composition, including radionuclides, antimicrobial agents, solubilizing agents or the like.

The kit may be a kit of two or more parts comprising any of the components exemplified above in suitable containers. For example, each container may be in the form of vials, bottles, squeeze bottles, jars, sealed sleeves, envelopes or pouches, tubes or blister packages or any other suitable form, provided the container preferably prevents premature mixing of components. Each of the different components may be provided separately, or some of the different components may be provided together (i.e. in the same container).

A container may also be a compartment or a chamber within a vial, a tube, a jar, or an envelope, or a sleeve, or a blister package or a bottle, provided that the contents of one compartment are not able to associate physically with the contents of another compartment prior to their deliberate mixing by a pharmacist or physician.

The kit or kit-of-parts may furthermore contain technical instructions with information on the administration and/or dosage of any of its components.

### Therapeutic and Diagnostic Methods and Uses

According to a further aspect, the present invention also provides the conjugate (compound) (including pharmaceutically acceptable salts, esters, solvates and radiolabeled complexes thereof), pharmaceutical composition or kit according to the present invention for use in medicine. Furthermore, the present invention also provides the conjugate or compound (including pharmaceutically acceptable salts, esters, solvates and radiolabeled complexes thereof), pharmaceutical composition or kit according to the present invention for use in diagnostics. Preferably, the conjugates (compounds), pharmaceutical compositions or kits of the invention are used for human medical purposes. Accordingly, the invention further encompasses the conjugates (compounds), pharmaceutical composition or kit of the invention for use as a medicament.

The inventive conjugates (compounds) are preferably capable of targeting prostate-specific membrane antigen (PSMA) in a selective manner. According to a specific aspect, the invention thus provides the inventive conjugates (compounds), pharmaceutical compositions or kits for use in a method of detecting the presence of cells and/or tissues expressing prostate-specific membrane antigen (PSMA).

PSMA is in particular expressed on malignant cancer cells. As used herein, the term “cancer” refers to a neoplasm, in particular a malignant neoplasm. A neoplasm is typically characterized by the uncontrolled and usually rapid proliferation of cells that tend to invade surrounding tissue and to metastasize to distant body sites. The term “neoplasm” encompasses benign and malignant neoplasms. Malignant neoplasms (cancers) are typically characterized by anaplasia, invasiveness, and/or metastasis; while benign neoplasms typically have none of those properties. The term “cancer” include neoplasms characterized by tumor growth (e.g., solid tumors) as well as other cancers, e.g. cancers of blood and lymphatic system.

Specifically, PSMA may be expressed, optionally in increased amounts, in prostate cancer cells, pancreatic cancer cells, renal cancer cells or bladder cancer cells.

According to a further specific aspect, the invention provides the inventive conjugate (compound) (including pharmaceutically acceptable salts, esters, solvates and radiolabeled complexes thereof), pharmaceutical composition or kit for use in a method of diagnosing, treating and/or preventing cancer, in particular prostate cancer, pancreatic cancer, renal cancer or bladder cancer.

The term “diagnosis” or “diagnosing” refers to act of identifying a disease from its signs and symptoms and/or as in the present case the analysis of biological markers (such as genes or proteins) indicative of the disease.

The term “treatment” or “treating” of a disease includes preventing or protecting against the disease (that is, causing the clinical symptoms not to develop); inhibiting the disease (i.e., arresting or suppressing the development of clinical symptoms; and/or relieving the disease (i.e., causing the regression of clinical symptoms). As will be appreciated, it is not always possible to distinguish between “preventing” and “suppressing” a disease or disorder since the ultimate inductive event or events may be unknown or latent. Accordingly, the term “prophylaxis” will be understood to constitute a type of “treatment” that encompasses both “preventing” and “suppressing.” The term “treatment” thus includes “prophylaxis”.

The term “subject”, “patient” or “individual” as used herein generally includes humans and non-human animals and preferably mammals (e.g., non-human primates, including marmosets, tamarins, spider monkeys, owl monkeys, vervet monkeys, squirrel monkeys, and baboons, macaques, chimpanzees, orangutans, gorillas; cows; horses; sheep; pigs; chicken; cats; dogs; mice; rat; rabbits; guinea pigs etc.), including chimeric and transgenic animals and disease models. In the context of the present invention, the term “subject” preferably refers a non-human primate or a human, most preferably a human.

The uses and methods described herein and relating to the diagnosis, treatment or prophylaxis of cancer, in particular prostate cancer, pancreatic cancer, renal cancer or bladder cancer, may preferably comprise the steps of (a) administering the inventive conjugate (including pharmaceutically acceptable salts, esters, solvates and radiolabeled complexes thereof), pharmaceutical composition or kit to a patient, and (b) obtaining a radiographic image from said patient.

The inventive conjugates (compounds), pharmaceutical compositions or kits are typically administered parenterally. Administration may preferably be accomplished systemically, for instance by intravenous (i.v.), subcutaneous, intramuscular or intradermal injection. Alternatively, administration may be accomplished locally, for instance by intra-tumoral injection.

The inventive conjugates (compounds), pharmaceutical compositions or kits may be administered to a subject in need thereof several times a day, daily, every other day, weekly, or monthly. Preferably, treatment, diagnosis or prophylaxis is effected with an effective dose of the inventive conjugates, pharmaceutical compositions or kits.

Effective doses of the inventive conjugates may be determined by routine experiments, e.g. by using animal models. Such models include, without implying any limitation, rabbit, sheep, mouse, rat, dog and non-human primate models. Therapeutic efficacy and toxicity of inventive conjugates or radiolabeled complexes can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. The data obtained from the cell culture assays and animal studies can be used in determining a dose range for use in humans. The dose of said conjugates lies preferably within a range of circulating concentrations that include the ED50 with little or no toxicity.

For instance, therapeutically or diagnostically effective doses of the inventive conjugates may range from about 0.001 mg to 10 mg, preferably from about 0.01 mg to 5 mg, more preferably from about 0.1 mg to 2 mg per dosage unit or from about 0.01 nmol to 1 mmol per dosage unit, in particular from 1 nmol to 1 mmol per dosage unit, preferably from 1 micromol to 1 mmol per dosage unit. It is also envisaged that therapeutically or diagnostically effective doses of the inventive conjugates (compounds) may range (per kg body weight) from about 0.01 mg/kg to 10 g/kg, preferably from about 0.05 mg/kg to 5 g/kg, more preferably from about 0.1 mg/kg to 2.5 g/kg. Advantageously, due to their favorable pharmacokinetic properties, the inventive conjugates may preferably be administered at lower doses than other PSMA ligands.

As established above, the inventive conjugates particularly lend themselves for theragnostic applications involving the targeting of PSMA-expressing cells. As used herein, the term “therangostic” includes “therapeutic-only”, “diagnostic-only” and “therapeutic and diagnostic” applications. In a further aspect, the present invention relates to an in vitro method of detecting the presence of cells and/or tissues expressing prostate-specific membrane antigen (PSMA) comprising (a) contacting said PSMA-expressing cells and/or tissues with the inventive conjugates (including pharmaceutically acceptable salts, esters, solvates and radiolabeled complexes thereof), pharmaceutical compositions or kits and (b) applying detection means, optionally radiographic imaging, to detect said cells and/or tissues.

In the in vivo and in vitro uses and methods of the present invention, radiographic imaging may be accomplished using any means and methods known in the art. Preferably, radiographic imaging may involve positron emission tomography (PET) or single-photon emission computed tomography (SPECT). The targeted cells or tissues detected by radiographic imaging of the inventive conjugate may preferably comprise (optionally cancerous) prostate cells or tissues, (optionally cancerous) spleen cells or tissues, or (optionally cancerous) kidney cells or tissues.

In the in vivo and in vitro uses and methods of the present invention, the presence of PSMA-expressing cells or tissues may be indicative of a prostate tumor (cell), a metastasized prostate tumor (cell), a renal tumor (cell), a pancreatic tumor (cell), a bladder tumor (cell), and combinations thereof. Hence, the inventive conjugates (including pharmaceutically acceptable salts, esters, solvates and radiolabeled complexes thereof), pharmaceutical compositions and kit may particularly be employed for diagnosis (and optionally treatment) of prostate cancer, renal cancer, pancreatic cancer, or bladder cancer.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows in Scheme 1 the synthesis of the Glutamate-Urea-Lysine Binding Motif for Ibu-DAB-PSMA.

FIG. 2 shows in Scheme 2 the synthesis of the Linker Area, Precursor for Ibu-Dab-PSMA.

FIG. 3 shows in Scheme 3 the synthesis of the DOTA-conjugated Precursor for Ibu-Dab-PSMA.

FIG. 4 shows in Scheme 4 the coupling of the additional linker moiety and albumin-binding entity for Ibu-DAB-PSMA.

FIG. 5 shows for Example 4 representative HPLC chromatograms of the ibuprofen-derivatized ¹⁷⁷Lu-PSMA-ligands. (A) Chromatogram of ¹⁷⁷Lu-Ibu-PSMA; (B) Chromatogram of ¹⁷⁷Lu-Ibu-Dβ-PSMA; (C) Chromatogram of ¹⁷⁷Lu-Ibu-Dα-PSMA; (D) Chromatogram of ¹⁷⁷Lu-Ibu-N-PSMA; (E) Chromatogram of ¹⁷⁷Lu-Ibu-DAB-PSMA. Retention times t_{R} are indicated in the figures.

FIG. 6 shows for Example 5 the n-Octanol/PBS distribution coefficients of ¹⁷⁷Lu-Ibu-PSMA, ¹⁷⁷Lu-Ibu-Dβ-PSMA, ¹⁷⁷Lu-Ibu-Dα-PSMA, ¹⁷⁷Lu-Ibu-N-PSMA and ¹⁷⁷Lu-Ibu-DAB-PSMA in comparison to the reference compound ¹⁷⁷Lu-PSMA-617. The experiments were performed three times (n=3) in quintuplicate.

FIG. 7 shows for Example 6 the data from ultrafiltration assays of ¹⁷⁷Lu-Ibu-PSMA, ¹⁷⁷Lu-Ibu-Dβ-PSMA, ¹⁷⁷Lu-Ibu-Dα-PSMA, ¹⁷⁷Lu-Ibu-N-PSMA and ¹⁷⁷Lu-Ibu-DAB-PSMA in comparison to ¹⁷⁷Lu-PSMA-617. (n=3)

FIG. 8 shows for Example 7 the uptake and internalization of ¹⁷⁷Lu-Ibu-PSMA, ¹⁷⁷Lu-Ibu-Dβ-PSMA, ¹⁷⁷Lu-Ibu-Dα-PSMA, ¹⁷⁷Lu-Ibu-N-PSMA and ¹⁷⁷Lu-Ibu-DAB-PSMA in comparison to ¹⁷⁷Lu-PSMA-617. (A) Data obtained in PSMA-positive PC-3 PIP cells (n=3). (B) Data obtained in PSMA-positive PC-3 flu cells (n=1).

FIG. 9 shows for Example 8 the biodistribution data of the five ibuprofen-derivatized radioligands and ¹⁷⁷Lu-PSMA-617 obtained in PC-3 PIP/flu tumor-bearing mice. (A) Biodistribution data obtained 4 h after injection of the radioligands; (B) Biodistribution data obtained 24 h after injection of the radioligands.

FIG. 10 shows for Example 8 tumor-to-background ratios at 4 h and 24 h after injection of the ¹⁷⁷Lu-PSMA-ligands. (A) Tumor-to-blood ratios, (B) tumor-to-liver ratios and (C) tumor-to-kidney ratios for all ¹⁷⁷Lu-Ibu-PSMA-ligands at 4 h and 24 h p.i.

FIG. 11 shows for Example 9 the whole-body activity measured in a dose calibrator at 0 h, 4 h, 24 h, 48 h and 72 h after injection of the respective radioligands. The activity measured right after injection was set as 100%. Data for comparative radioligands ¹⁷⁷Lu-PSMA-ALB-53/56 and ¹⁷⁷Lu-PSMA-617 are included in this graph for comparison. The data points present the average of two mice which were injected with the same radioligand (n=2).

FIG. 12 shows for Example 10 SPECT/CT images obtained 4 h after injection of the ¹⁷⁷Lu-PSMA-ligands shown as maximum intensity projections (MIP). (A)¹⁷⁷Lu-Ibu-PSMA; (B) ¹⁷⁷Lu-Ibu-Dβ-PSMA; (C)¹⁷⁷Lu-Ibu-Dβ-PSMA; (D) ¹⁷⁷Lu-Ibu-N-PSMA; (E) ¹⁷⁷Lu-Ibu-DAB-PSMA. PSMA+=PSMA-positive PC-3 PIP tumor xenograft; PSMA−=PSMA-negative PC-3 flu tumor xenograft; Ki=Kidney; Bl=urinary bladder.

FIG. 13 shows a scheme presenting the coupling of the ibuprofen moiety to Precursor 1 (including the PSMA binding entity and a DOTA chelator) for synthesizing Ibu-sPSMA.

FIG. 14 Representative HPLC chromatogram of ¹⁷⁷Lu-Ibu-sPSMA. The retention time tr is indicated in the figure.

FIG. 15 Radiolytic stability presented as percentage of intact ¹⁷⁷Lu-Ibu-sPSMA up to 24 h. (A)¹⁷⁷Lu-Ibu-sPSMA incubated without L-ascorbic acid; (B) ¹⁷⁷Lu-Ibu-sPSMA incubated with L-ascorbic acid (average±SD, n=3). ¹⁷⁷Lu-Ibu-sPSMA was significantly more stable than ¹⁷⁷Lu-PSMA-617 and all other ibuprofen-derivatized PSMA radioligands. The stability of ¹⁷⁷Lu-Ibu-sPSMA was comparable to the stability of ¹⁷⁷Lu-PSMA-ALB-56.

FIG. 16 Data from ultrafiltration assays of ¹⁷⁷Lu-Ibu-sPSMA in comparison to ¹⁷⁷Lu-PSMA-617. (n=3)

FIG. 17 Uptake and internalization of ¹⁷⁷Lu-Ibu-sPSMA in comparison to ¹⁷⁷Lu-PSMA-617. (A) Data obtained in PSMA-positive PC-3 PIP cells (n=3). (B) Data obtained in PSMA-negative PC-3 flu cells (n=3).

FIG. 18 Graph showing biodistribution data of ¹⁷⁷Lu-Ibu-PSMA, ¹⁷⁷Lu-Ibu-DAB-PSMA, ¹⁷⁷Lu-Ibu-sPSMA and ¹⁷⁷Lu-Ibu-PSMA-617 obtained in PC-3 PIP/flu tumor-bearing mice. (A) Biodistribution data obtained 1 h after injection of the radioligands; (B) Biodistribution data obtained 4 h after injection of the radioligands; (C) Biodistribution data obtained 24 h after injection of the radioligands and (D) biodistribution data obtained 96 h after injection of the radioligands.

FIG. 19 The graphs show tumor-to-background ratios at 1 h, 4 h, 24 h and 96 h after injection of ¹⁷⁷Lu-Ibu-sPSMA in comparison to ¹⁷⁷Lu-Ibu-PSMA and ¹⁷⁷Lu-Ibu-DAB-PSMA. (A) Tumor-to-blood ratios, (B) tumor-to-kidney ratios and (C) tumor-to-liver ratios.

FIG. 20 Whole-body activity measured in a dose calibrator at various time-points after injection. The activity measured right after injection was set as 100%. Published data of ¹⁷⁷Lu-PSMA-617 is included in the graphs for comparison. The data points present the average of two mice, which were injected with the same radioligand (n=2-3). (A) The graph shows data of all radioligands; (B) the graph shows data of ¹⁷⁷Lu-Ibu-PSMA, ¹⁷⁷Lu-Ibu-DAB-PSMA, ¹⁷⁷Lu-PSMA-617 and ¹⁷⁷Lu-PSMA-ALB-56 for better visualization of the single excretion curves.

FIG. 21 SPECT/CT images obtained after injection of the ¹⁷⁷Lu-Ibu-sPSMA shown as maximum intensity projections (MIP). (A) SPECT/CT image acquired 4 h p.i.; (B) SPECT/CT image acquired 24 h p.i. PSMA+=PSMA-positive PC-3 PIP tumor xenograft; PSMA−=PSMA-negative PC-3 flu tumor xenograft; Ki=Kidney; Bl=urinary bladder.

FIG. 22 Relative tumor growth of control mice and mice treated with (a) lower quantity of activity (2 MBq, 1 nmol per mouse) or (b) higher quantity of activity (5 MBq, 1 nmol per mouse). Each group of mice was injected with only vehicle (saline) (●), ¹⁷⁷Lu-Ibu-DAB-PSMA (▪), ¹⁷⁷Lu-PSMA-617 (▴) and ¹⁷⁷Lu-PSMA-ALB-56 (▾), respectively, six days after tumor cell inoculation (average±SD, n=6-12). Average relative tumor volumes of each group are shown until the first mouse reached an endpoint.

FIG. 23 Kaplan-Meier plot with survival curves of mice of each group (n=6-12). Control mice and mice treated with (a) lower quantity of injected activity (2 MBq, 1 nmol per mouse) and (b) higher quantity of injected activity (5 MBq, 1 nmol per mouse). Untreated control mice (-), ¹⁷⁷Lu-Ibu-DAB-PSMA (---); ¹⁷⁷Lu-PSMA-617 (-••-••) and ¹⁷⁷Lu-PSMA-ALB-56 (•••).

FIG. 24 Relative body weight (RBW) of control mice and mice treated with (a) lower quantity of injected activity (2 MBq, 1 nmol per mouse) and (b) higher quantity of injected activity (5 MBq, 1 nmol per mouse). Average RBW of mice injected with only vehicle (saline) (●), ¹⁷⁷Lu-Ibu-DAB-PSMA (▪), ¹⁷⁷Lu-PSMA-617 (▴) and ¹⁷⁷Lu-PSMA-ALB-56 (▾), respectively. Average RBW of each group shown until the first mouse reached an endpoint.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Structural Design of Exemplified PSMA-Ligands

In order to identify PSMA-ligands, which provide a balance between (i) the binding of the radioligand to albumin in order to achieve an optimal tissue distribution profile with high tumor uptake and (ii) blood activity levels that are not extensively high, which would result in a risk for undesired side effects to healthy tissue, the following five ibuprofen-derivatized PSMA-ligands were designed (Ibu-PSMA, Ibu-Dα-PSMA, Ibu-Dβ-PSMA, Ibu-N-PSMA and Ibu-DAB-PSMA):

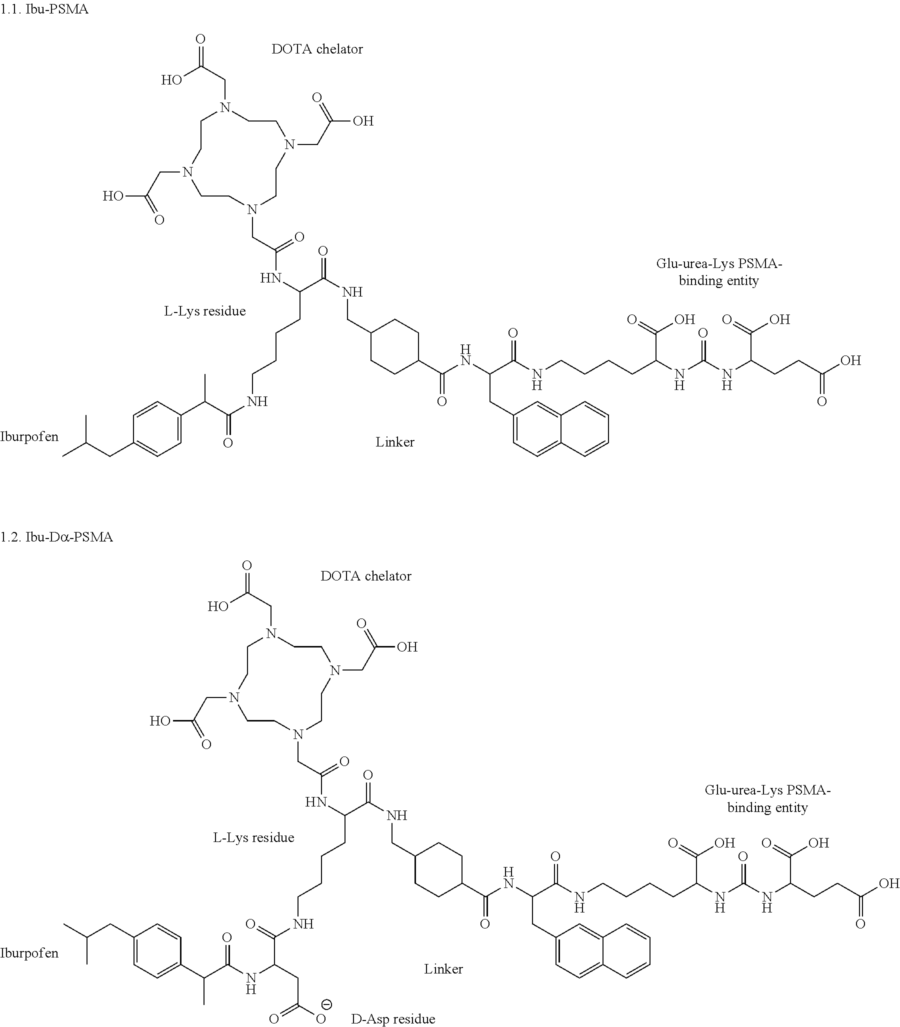

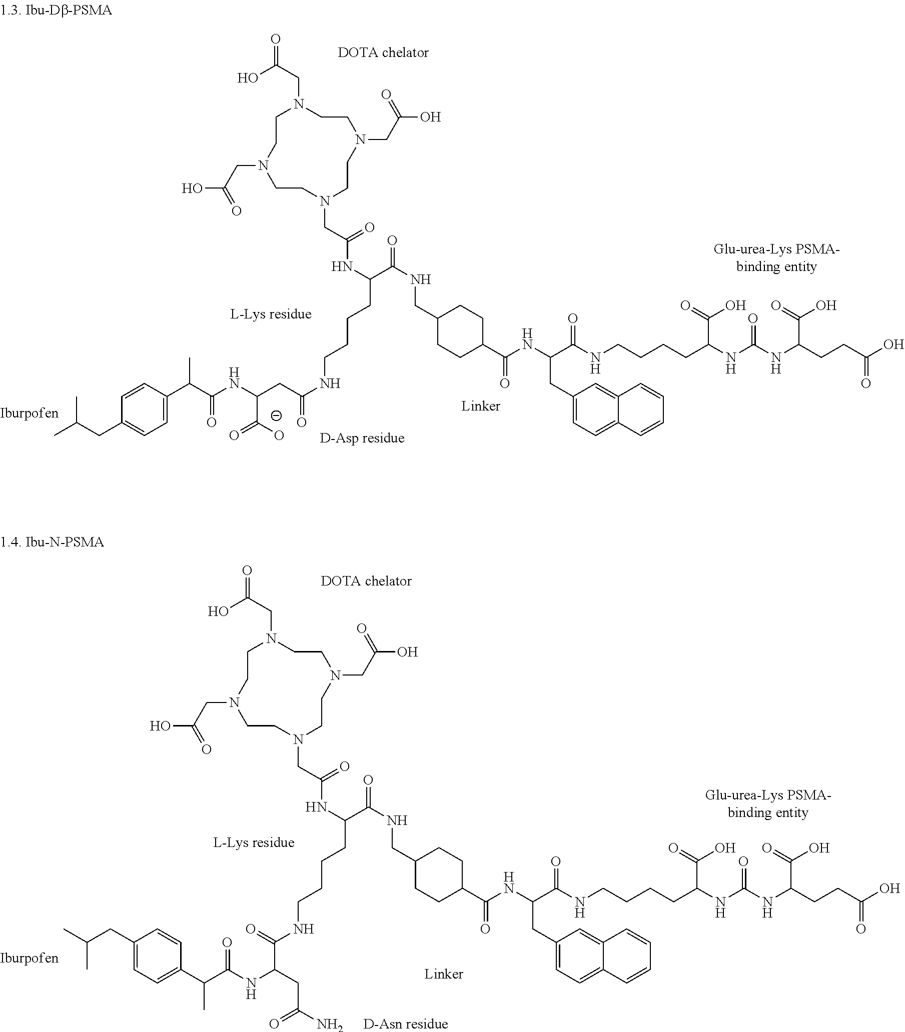

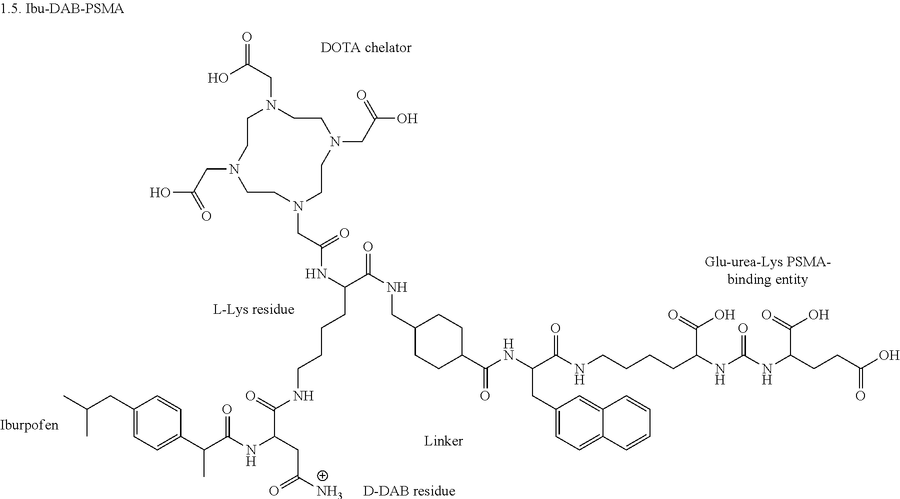 [SMILES: CC(C)Cc1ccc(C(C)C(=O)NC(CC([NH3+])=O)C(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NC(=O)NC(CCC(=O)O)C(=O)O)C(=O)O)CC2)cc1 ; "D-DAB residue"]

The simplest design of an ibuprofen-derivatized PSMA-ligand is Ibu-PSMA. It was designed by introducing the albumin binder ibuprofen without any additional spacer entity by conjugating ibuprofen directly to the lysine residue. In Ibu-Dα-PSMA and Ibu-Dβ-PSMA an additional spacer based on D-aspartic acid (D-Asp, D) was used (in addition to the L-Lys residue) to introduce an additional negative charge to the construct. D-Asp was conjugated either via the α-carboxyl group to obtain Ibu-Dα-PSMA or via the β-carboxyl group to obtain Ibu-Dβ-PSMA. In Ibu-N-PSMA a different additional spacer entity based on D-asparagine (D-Asn, N) was employed acting as neutral entity (in addition to the L-Lys residue). Finally, the design of Ibu-DAB-PSMA was based on the use of D-diaminobutyric acid (DAB) as additional spacer entity (in addition to the L-Lys residue) to introduce an additional positive charge to the construct.

### 1.6. Ibu-sPSMA:

Ibu-sPSMA was designed in analogy to Ibu-PSMA. In contrast to Ibu-PSMA, in which the ibuprofen moiety was connected via a lysine side chain, the shorter L-2,4-diaminobutyric acid (L-DAB) was used as connecting unit.

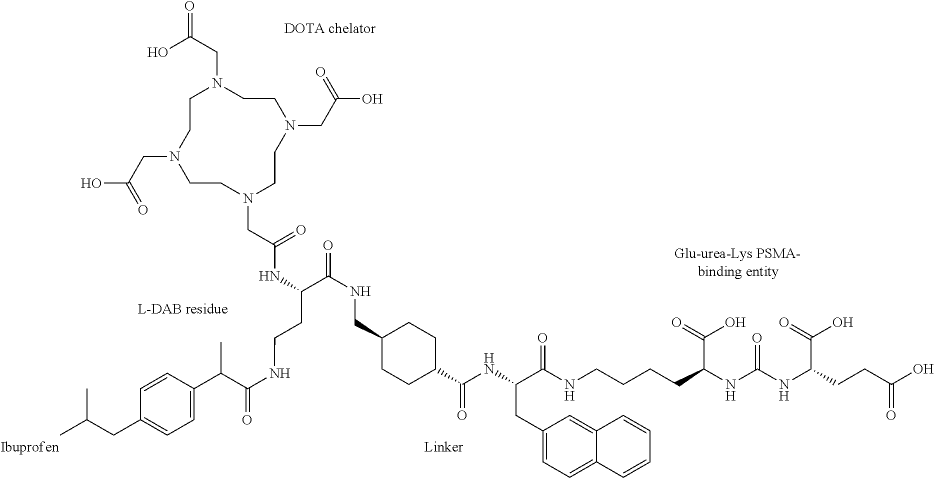

### Example 2: Chemical Synthesis of the Exemplified PSMA-Ligands

### 2.1. Synthetic Strategy and Analysis of the PSMA-Ligands

All five suggested PSMA-ligands with an albumin-binding moiety were synthesized via a solid-phase platform as previously reported for the synthesis of other PSMA-ligands (Umbricht, C. A.; Benesova, M.; Schibli, R.; Müller, C. Preclinical development of novel PSMA-targeting radioligands: modulation of albumin-binding properties to improve prostate cancer therapy. Mol Pharm 2018, Mol Pharm 2018, 15, (6), 2297-2306). This technique revealed to be useful for the development of the described ibuprofen-derivatized PSMA-ligands. A multistep synthesis (17 steps for Ibu-PSMA and 19 steps Ibu-Dα-PSMA, Ibu-Dβ-PSMA, Ibu-N-PSMA and Ibu-DAB-PSMA) provided these ligands in isolated overall yields of ≥2.8% after HPLC purification. The ligands were characterized by analytical RP-HPLC and MALDI-MS, respectively. The chemical purity of the compounds was ≥99.2%. Analytical data are presented in Table 1.

TABLE 1
Analytical data of the PSMA-ligands: Ibu-PSMA, Ibu-Dα-PSMA,
Ibu-Dβ-PSMA, Ibu-N-PSMA and Ibu-DAB-PSMA.

| | | | | | | Chemical |
|---|---|---|---|---|---|---|
| | Chemical | MW | | Amount | Yield | purity^{b} |
| Compound | formula | [g/mol] | m/z^{a} | [mg] | [%] | [%] |
| Ibu-PSMA | C₆₈H₉₉N₁₁O₁₈ | 1358.72 | 1358.60 | 3.8 | 2.8 | >99.2 |
| Ibu-Dα-PSMA | C₇₂H₁₀₄N₁₂O₂₁ | 1473.75 | 1473.69 | 8.4 | 5.7 | >99.5 |
| Ibu-Dβ-PSMA | C₇₂H₁₀₄N₁₂O₂₁ | 1473.75 | 1473.69 | 4.7 | 3.2 | >99.6 |
| Ibu-N-PSMA | C₇₂H₁₀₅N₁₃O₂₀ | 1472.76 | 1472.70 | 12.0 | 8.2 | >99.7 |
| Ibu-DAB-PSMA | C₇₂H₁₀₇N₁₃O₁₉ | 1458.78 | 1458.72 | 21.3 | 14.6 | >99.5 |

^{a}m/z-peak of the unlabeled ligand obtained by mass spectrometry;
^{b}Determined by analytical HPLC, λ = 254 nm;

### 2.2. Synthesis of Precursor 1

The PSMA-targeting urea-based PSMA-binding entity—L-Glu-NH—CO—NH-L-Lys—was prepared on a 2-chlotrotrityl chloride (2-CT) resin in analogy to the method described by Eder eta/. (Eder, M.; Schäfer, M.; Bauder-Wust, U.; Hull, W. E.; Wängler, C.; Mier, W.; Haberkorn, U.; Eisenhut, M. ⁶⁸Ga-complex lipophilicity and the targeting property of a urea-based PSMA inhibitor for PET imaging. Bioconjug Chem 2012, 23, (4), 688-97). The linker area consisting of a 2-naphthyl-L-Ala and a trans-cyclohexyl moiety was synthesized as previously reported by Benes̆ová et al. (Benesova, M.; Schäfer, M.; Bauder-Wüst, U.; Afshar-Oromieh, A.; Kratochwil, C.; Mier, W.; Haberkorn, U.; Kopka, K.; Eder, M. Preclinical evaluation of a tailor-made DOTA-conjugated PSMA inhibitor with optimized linker moiety for imaging and endoradiotherapy of prostate cancer. J Nucl Med 2015, 56, (6), 914-20). The conjugation of the DOTA-chelator conjugated via a Nα-amino-L-Lys to above described construct was previously reported by Umbricht et al. (Umbricht, C. A.; Benesova, M.; Schibli, R.; Müller, C. Preclinical development of novel PSMA-targeting radioligands: modulation of albumin-binding properties to improve prostate cancer therapy. Mol Pharm 2018, Mol Pharm 2018, 15, (6), 2297-2306).

The following resin-immobilized precursor was used as the basis for the synthesis of the PSMA-ligands (“precursor 1”):

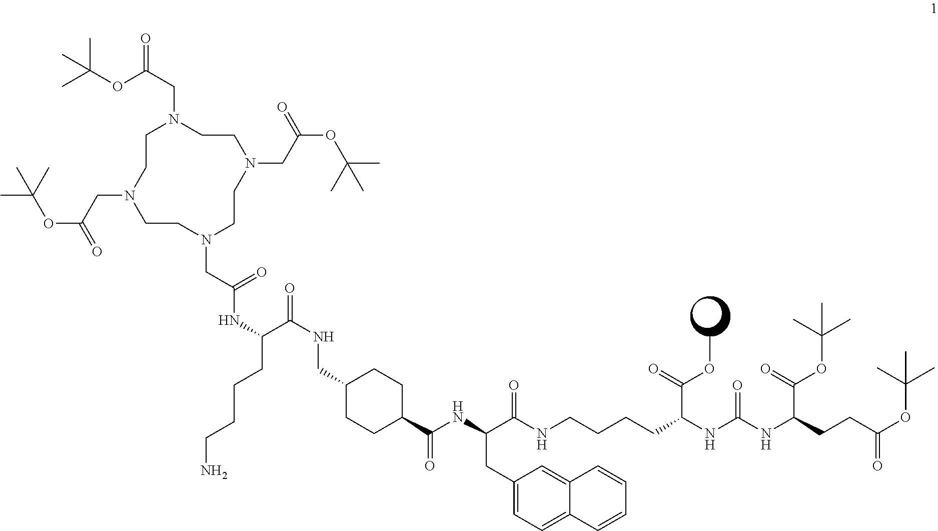 [SMILES: COC(=O)[C@@H](CCCCNC(=O)[C@@H](Cc1ccc2ccccc2c1)NC(=O)[C@H]1CC[C@H](CNC(=O)[C@H](CCCCN)NC(=O)CN2CCN(CC(=O)OC(C)(C)C)CCN(CC(=O)OC(C)(C)C)CCN(CC(=O)OC(C)(C)C)CC2)CC1)NC(=O)N[C@H](CCC(=O)OC(C)(C)C)C(=O)OC(C)(C)C]

Precursor 1 is based on the PSMA-binding entity and a DOTA-chelator. This precursor was employed for the synthesis of the five exemplified ligands Ibu-PSMA, Ibu-Dα-PSMA, Ibu-Dβ-PSMA, Ibu-N-PSMA and Ibu-DAB-PSMA. The free amino group of the lysine side chain was used for conjugation of ibuprofen which was connected directly or via an amino acid entity.

### 2.3. Synthesis of Ibu-PSMA

The synthesis of Ibu-PSMA was performed by coupling the albumin-binding ibuprofen to the resin-immobilized precursor 1. The resin was swelled in anhydrous dichloromethane (DCM, Acros Organics) for 45 min and subsequently conditioned in N,N-dimethylformamide (DMF, Acros Organics). Relative to the resin-immobilized precursor 1 (0.10 mmol), 4.0-6.0 equiv 2-(4-(2-methylpropyl)phenyl)propanoic acid (ibuprofen; Sigma Aldrich; 0.400-0.600 mmol) were activated using 3.96 equiv N,N,N′,N′-tetramethyl-O-(1H-benzotriazol-1-yl)-uronium hexafluoro-phosphate (HBTU; Sigma Aldrich, 0.396-0.594 mmol) in the presence of 4.0-6.0 equiv DIPEA (N,N-diisopropylethylamine, Sigma Aldrich, 0.400-0.600 mmol) in anhydrous DMF. Two minutes after the addition of DIPEA, the activated solution was added to the precursor 1 and agitated up to 2 h. The resin was washed with DMF, DCM and diethyl ether, respectively, and dried under reduced pressure. The product was cleaved from the resin and subsequently deprotected within 3-6 h using a mixture consisting of trifluoroacetic acid (TFA, Sigma Aldrich), triisopropylsilane (TIPS, Sigma Aldrich) and Milli-Q water in a ratio of 95:2.5:2.5 (v/v). TFA was evaporated, the crude compound dissolved in acetonitrile (ACN, VWR Chemicals) and Milli-Q water in a ratio of 1:2 (v/v) and purified by RP-HPLC to yield Ibu-PSMA.

### 2.4. Synthesis of Ibu-Dα-PSMA

The additional spacer entity consisting of D-aspartic acid (D-Asp) was conjugated to NE-L-lysine of the precursor 1 before coupling the ibuprofen. The resin-immobilized precursor 1 was pre-swollen in DCM and conditioned in DMF as described above. Relative to precursor 1 (0.100 mmol), 4.0 equiv Fmoc and t-Bu protected D-Asp (Fmoc-D-Asp(O-t-Bu)-OH, Sigma Aldrich, 0.400 mmol) were activated using 3.96 equiv HBTU (0.396 mmol) in the presence of 4.0 equiv DIPEA (0.400 mmol) in anhydrous DMF. Two minutes after the addition of DIPEA, the activated solution was added to precursor 1 and agitated up to 2 h. The resin was washed with DMF. The Na-Fmoc-protecting group was cleaved by agitating with a mixture of DMF and piperidine (Fluka) in a ratio of 1:1 (v/v) twice for 5 min. The resin was again washed with DMF. Ibuprofen (4.0-6.0 equiv; 0.400-0.600 mmol) was activated using 3.96 equiv HBTU (0.396-0.594 mmol) in the presence of 4.0-6.0 equiv DIPEA (0.400-0.600 mmol) in anhydrous DMF. Two minutes after the addition of DIPEA, the activated solution was added to the resin and agitated up to 2 h. Subsequently, resin was washed with DMF, DCM and diethyl ether, respectively, and dried under reduced pressure. The product was cleaved from the resin and simultaneously deprotected with a mixture consisting of TFA, TIPS and water in a ratio of 95:2.5:2.5 (v/v) within 3-6 h. TFA was evaporated, The crude compound dissolved in acetonitrile (ACN, VWR Chemicals) and Milli-Q water in a ratio of 1:2 (v/v) and purified by RP-HPLC to yield Ibu-Dα-PSMA.

### 2.5. Synthesis of Ibu-Dβ-PSMA

The additional spacer entity consisting of D-aspartic acid (D-Asp) was conjugated to Nε-L-lysine of the precursor 1 before coupling the ibuprofen. The resin-immobilized precursor 1 was pre-swollen in DCM and conditioned in DMF as described above. Relative to precursor 1 (0.100 mmol), 4.0 equiv of Fmoc and t-Bu protected D-Asp (Fmoc-D-Asp-O-t-Bu, Merck group, 0.400 mmol) were activated using 3.96 equiv HBTU (0.396 mmol) in the presence of 4.0 equiv DIPEA (0.400 mmol) in anhydrous DMF. Two minutes after the addition of DIPEA, the activated solution was added to the precursor 1 and agitated up to 2 h. The resin was washed with DMF and the Na-Fmoc-protecting group was cleaved by agitating with a mixture of DMF and piperidine (Fluka) in a ratio of 1:1 (v/v) twice for 5 min. The resin was again washed with DMF. Ibuprofen (4.0-6.0 equiv; 0.400-0.600 mmol) was activated using 3.96 equiv HBTU (0.396-0.594 mmol) in the presence of 4.0-6.0 equiv DIPEA (0.400-0.600 mmol) in anhydrous DMF. Two minutes after the addition of DIPEA, the activated solution was added to the resin and agitated up to 2 h. Subsequently, resin was washed with DMF, DCM and diethyl ether, respectively, and dried under reduced pressure. The product was cleaved from the resin and simultaneously deprotected with a mixture consisting of TFA, TIPS and water in a ratio of 95:2.5:2.5 (v/v) within 3-6 h. TFA was evaporated, the crude compound dissolved in acetonitrile (ACN, VWR Chemicals) and Milli-Q water in a ratio of 1:2 (v/v,) and purified by RP-HPLC to yield Ibu-Dβ-PSMA.

### 2.6. Synthesis of Ibu-N-PSMA

The additional spacer entity consisting of D-asparagine (D-Asn) was conjugated to Nε-L-lysine of the precursor 1 before coupling the ibuprofen. The resin-immobilized precursor 1 was pre-swollen in DCM and conditioned in DMF as described above. Relative to precursor 1 (0.100 mmol), 4.0 equiv of Fmoc and Trt (trityl) protected D-asparagine (Fmoc-D-Asn(Trt)-OH, Sigma Aldrich, 0.400 mmol) were activated using 3.96 equiv HBTU (0.396 mmol) in the presence of 4.0 equiv DIPEA (0.400 mmol) in anhydrous DMF. Two minutes after the addition of DIPEA, the activated solution was added to the precursor 1 and agitated up to 3 h. The resin was washed with DMF and and the Nα-Fmoc-protecting group was cleaved by agitating with a mixture of DMF and piperidine (Fluka) in a ratio of 1:1 (v/v) twice for 5 min. The resin was again washed with DMF. Ibuprofen (4.0-6.0 equiv; 0.400-0.600 mmol) were activated using 3.96 equiv HBTU (0.396-0.594 mmol) in the presence of 4.0-6.0 equiv DIPEA (0.400-0.600 mmol) in anhydrous DMF. Two minutes after the addition of DIPEA, the activated solution was added to the resin and agitated up to 2 h. Subsequently, resin was washed with DMF, DCM and diethyl ether, respectively, and dried under reduced pressure. The product was cleaved from the resin with a mixture consisting of TFA, TIPS and water in a ratio of 95:2.5:2.5 (v/v) within 3-6 h. The t-Bu-protecting groups and the additional Trt-protecting group were cleaved simultaneously. TFA was evaporated, the crude compound dissolved in acetonitrile (ACN, VWR Chemicals) and Milli-Q water in a ratio of 1:2 (v/v) and purified by RP-HPLC to yield Ibu-N-PSMA.

### 2.7. Synthesis of Ibu-DAB-PSMA

The additional spacer entity consisting of D-diaminobutyric acid was conjugated to Nε-L-lysine of the precursor 1 before coupling the ibuprofen. The resin-immobilized precursor 1 was pre-swollen in DCM and conditioned in DMF as described above. Relative to precursor 1 (0.100 mmol), 4.0 equiv of Fmoc and Boc (tert-Butyloxycarbonyl) protected D-diaminobutyric acid (DAB; Fmoc-D-Dab(Boc)-OH, Iris Biotech, 0.400 mmol) were activated using 3.96 equiv HBTU (0.396 mmol) in the presence of 4.0 equiv DIPEA (0.400 mmol) in anhydrous DMF. Two minutes after the addition of DIPEA, the activated solution was added to the precursor 1 and agitated up to 3.5 h. The resin was washed with DMF and the Nα-Fmoc-protecting group was cleaved by agitating with a mixture of DMF and piperidine (Fluka) in a ratio of 1:1 (v/v) twice for 5 min. The resin was again washed with DMF. Ibuprofen (4.0-6.0 equiv; 0.400-0.600 mmol) were activated using 3.96 equiv HBTU (0.396-0.594 mmol) in the presence of 4.0-6.0 equiv DIPEA (0.400-0.600 mmol) in anhydrous DMF. Two minutes after the addition of DIPEA, the activated solution was added to the resin and agitated up to 2 h. Subsequently, the resin was washed with DMF, DCM and diethyl ether, respectively, and dried under reduced pressure. The product was cleaved from the resin with a mixture consisting of TFA, TIPS and water in a ratio of 95:2.5:2.5 (v/v) within 3-6 h. The t-Bu-protecting groups and the additional Boc-protecting group were cleaved simultaneously. TFA was evaporated, the crude compound dissolved in acetonitrile (ACN, VWR Chemicals) and Milli-Q water in a ratio of 1:2 (v/v) and purified by RP-HPLC to yield Ibu-DAB-PSMA.

### 2.8. Synthesis of Ibu-sPSMA

In analogy to the other ibuprofen-bearing PSMA ligands, Ibu-sPSMA was synthesized via a solid-phase platform as previously reported (see also section 2 above) for the synthesis of other PSMA-ligands (Umbricht, C. A.; Mol Pharm 2018, 15, (6):2297-2306). A multistep synthesis (17 steps) provided this ligand in an isolated overall yield of ≥14% after HPLC purification.

### 2.8.1. Synthesis of Precursor 1

The PSMA-targeting urea-based PSMA-binding entity—L-Glu-NH—CO—NH-L-Lys—was prepared on a 2-chlotrotrityl chloride (2-CT) resin in analogy to the method described by Eder et al. (Bioconjug Chem 2012, 23, (4), 688-97), see also section 2. above. The linker area consisting of a 2-naphthyl-L-Ala and a trans-cyclohexyl moiety was synthesized as previously reported by Benes̆ová et al. (J Nucl Med 2015, 56, (6), 914-20). In this case, however, a different precursor than for the other Ibu-PSMA ligands was used. The linker entity L-diaminobutyric acid was by two carbon atoms shorter as compared to L-lysine, which was used as linker for the synthesis of Ibu-PSMA. The conjugation of the DOTA-chelator to above described construct was previously reported by Umbricht et al. (Mol Pharm 2018, 15, (6):2297-2306).

The following resin-immobilized precursor (precursor 1)

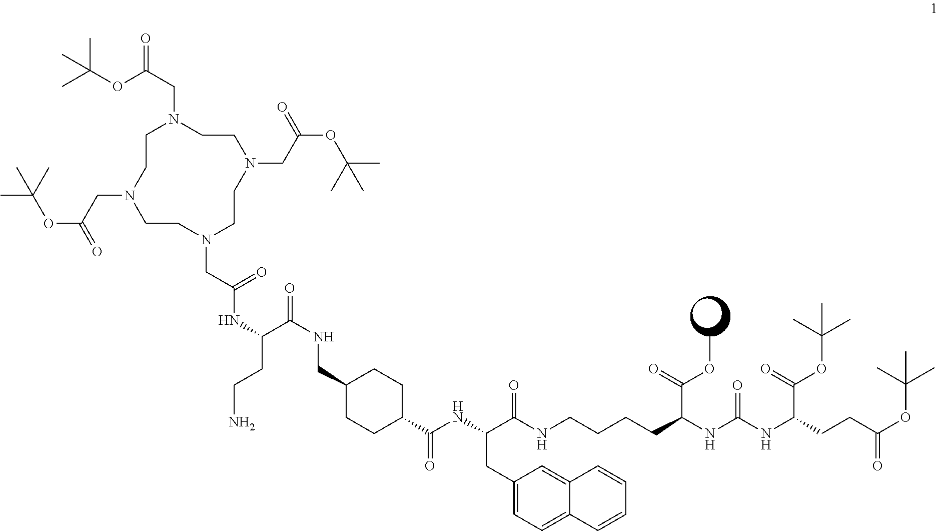 [SMILES: COC(=O)[C@H](CCCCNC(=O)[C@H](Cc1ccc2ccccc2c1)NC(=O)[C@H]1CC[C@H](CNC(=O)[C@H](CCN)NC(=O)CN2CCN(CC(=O)OC(C)(C)C)CCN(CC(=O)OC(C)(C)C)CCN(CC(=O)OC(C)(C)C)CC2)CC1)NC(=O)N[C@@H](CCC(=O)OC(C)(C)C)C(=O)OC(C)(C)C]

was used as the basis for the synthesis of the Ibu-sPSMA. Precursor 1 is based on the PSMA-binding entity and a DOTA-chelator. This precursor incorporated a shorter connecting entity than employed for other Ibu-PSMA ligands, e.g. Ibu-PSMA.

### 2.8.2. Synthesis of Ibu-sPSMA

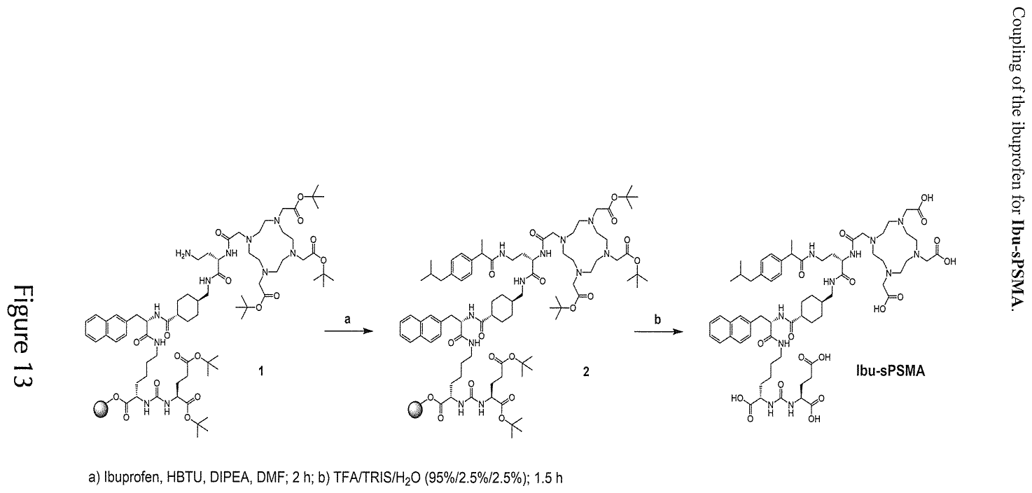

The synthesis of Ibu-sPSMA was performed by coupling the albumin-binding ibuprofen to the resin-immobilized precursor 1 (FIG. 13). The free γ-amino group of the diaminobutyric acid side chain was used for conjugation of ibuprofen. The resin was swelled in anhydrous dichloromethane (DCM, Acros Organics) for 45 min and subsequently conditioned in N,N-dimethylformamide (DMF, Acros Organics). Relative to the resin-immobilized precursor 1 (0.10 mmol), 6.0 equiv 2-(4-(2-methylpropyl)phenyl)propanoic acid (ibuprofen; Sigma Aldrich; 0.60 mmol) were activated using 5.94 equiv N,N,N′,N′-tetramethyl-O-(1H-benzotriazol-1-yl)-uronium hexafluoro-phosphate (HBTU; Sigma Aldrich, 0.59 mmol) in the presence of 8.0 equiv DIPEA (N,N-diisopropylethylamine, Sigma Aldrich, 0.80 mmol) in anhydrous DMF. Two minutes after the addition of DIPEA, the activated solution was added to the precursor 1 and agitated up to 2 h to yield resin-immobilized compound 2 (FIG. 13). The resin was washed with DMF, DCM and diethyl ether, respectively, and dried under reduced pressure. The product was cleaved from the resin and subsequently deprotected within 2 h using a mixture consisting of trifluoroacetic acid (TFA, Sigma Aldrich), triisopropylsilane (TIPS, Sigma Aldrich) and Milli-Q water in a ratio of 95:2.5:2.5 (v/v) to give the crude product (FIG. 13). TFA was evaporated, the crude compound dissolved in acetonitrile (ACN, VWR Chemicals) and Milli-Q water in a ratio of 1:2 (v/v) and purified by HPLC to yield pure Ibu-sPSMA.

The ligand was characterized by analytical HPLC and MALDI-MS, respectively. The chemical purity of the compound was ≥99%. Analytical data are presented in Table 2.

TABLE 2
Analytical data of Ibu-sPSMA.

| | | | | | | Chemical |
|---|---|---|---|---|---|---|
| | Chemical | MW | | Amount | Yield | purity^{b} |
| Compound | formula | [g/mol] | m/z^{a} | [mg] | [%] | [%] |
| Ibu-sPSMA | C₆₆H₉₅N₁₁O₁₈ | 1330.55 | 1330.69 | 28 | 15 | >99 |

^{a}m/z-peak of the unlabeled ligand obtained by mass spectrometry;
^{b}Determined by analytical HPLC, λ = 254 nm;

### 2.9. Synthesis of the Compound Lbu-DAB-PSMA as an Example

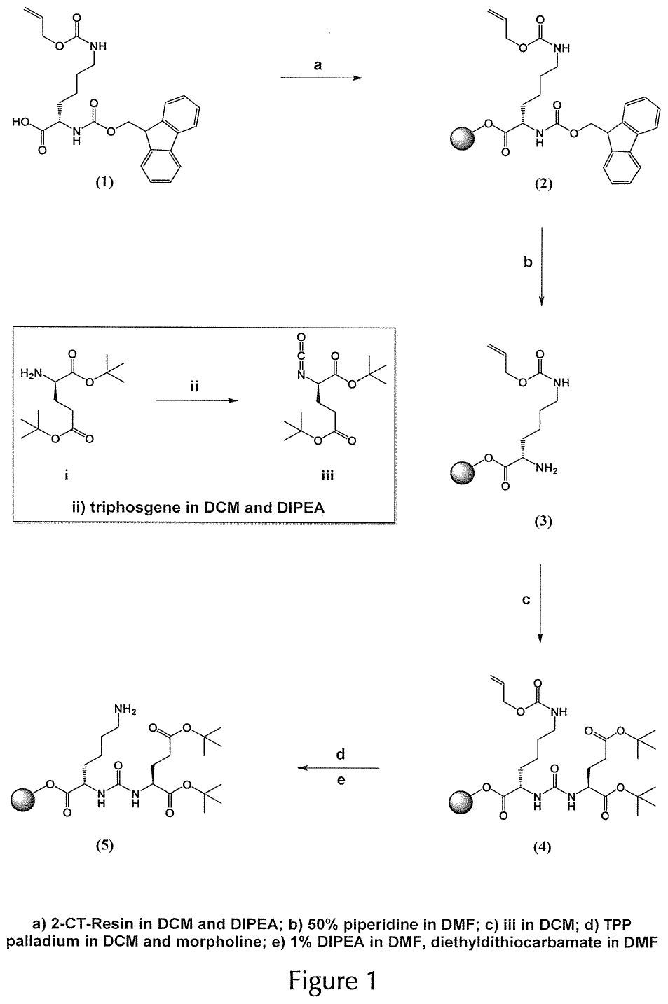

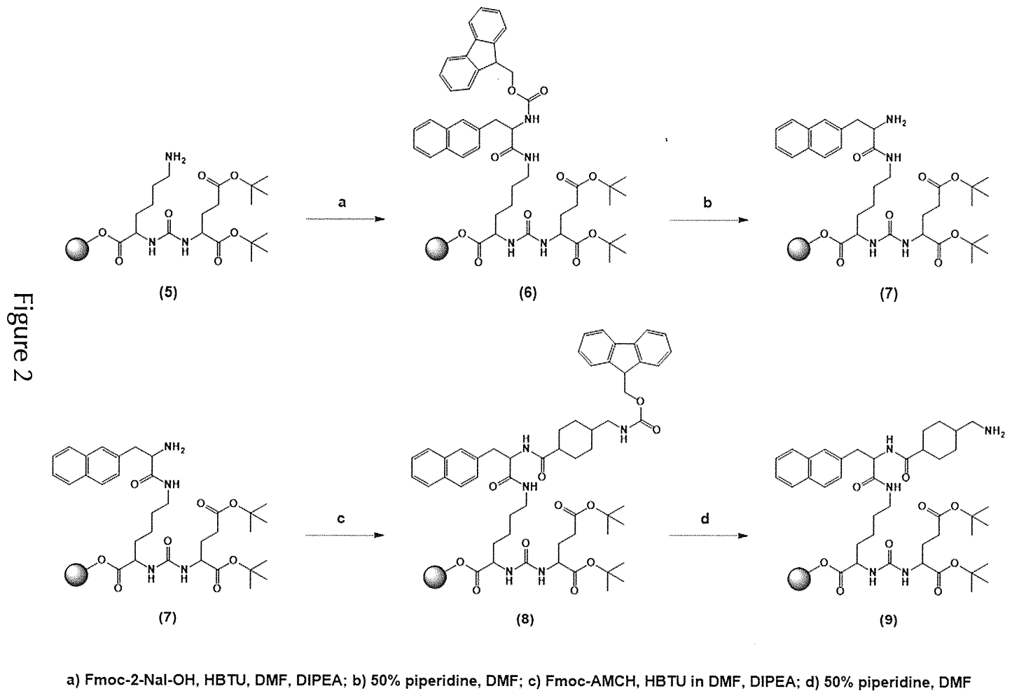

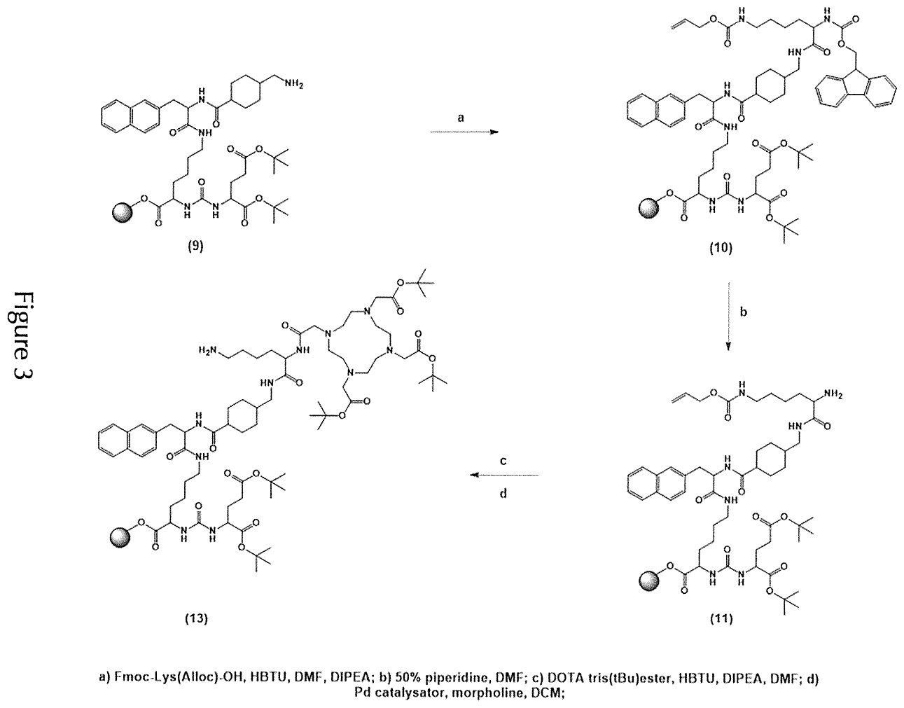

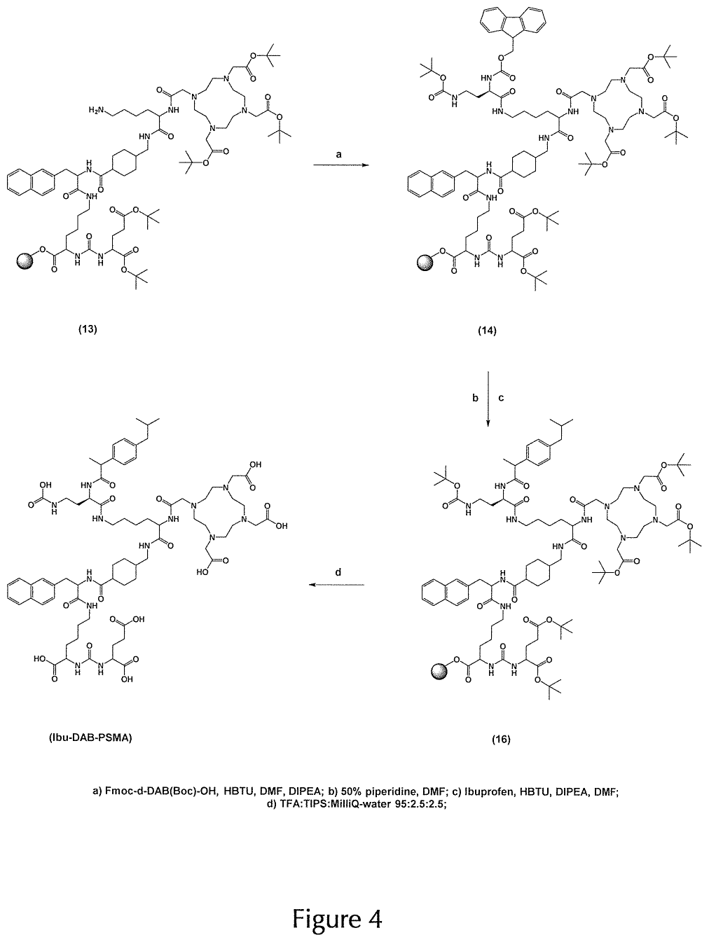

The synthesis schemes 1-4, which are shown in FIGS. 1-4, respectively, show the details of the synthesis of the compound Ibu-DAB-PSMA as an example. Synthesis of the other exemplified compounds was performed in a similar manner.

### Example 4: Radiolabeling and Stability

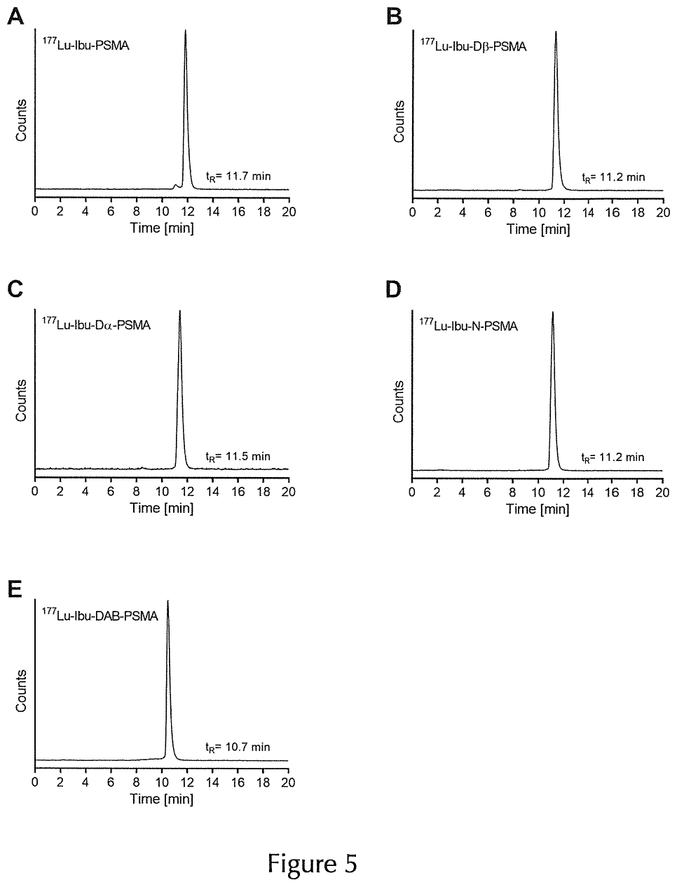

The stock solution of prior art PSMA-ligand PSMA-617 (ABX GmbH, Radeberg, Germany) was prepared by dilution of the ligand in MilliQ water to a final concentration of 1 mM. Ibu-PSMA, Ibu-Dα-PSMA, Ibu-Dβ-PSMA, Ibu-N-PSMA and Ibu-DAB-PSMA were diluted in Milli-Q water/sodium acetate (0.5 M, pH 8) to obtain a final concentration of 1 mM. All PSMA-ligands were labeled with ¹⁷⁷Lu (no-carrier added ¹⁷⁷Lu in 0.05 M HCl; Isotope Technologies Garching ITG GmbH, Germany) in a 1:5 (v/v) mixture of sodium acetate (0.5 M, pH 8) and HCl (0.05 M, pH ˜1) at pH ˜4.5. The PSMA-ligands were labeled with ¹⁷⁷Lu at specific activities between 5-50 MBq/nmol, depending on the experiment to be performed. The reaction mixture was incubated for 10 min at 95° C., followed by a quality control using RP-HPLC with a C-18 reversed-phase column (Xterra™ MS, C18, 5 μm, 150×4.6 mm; Waters). The mobile phase consisted of MilliQ water containing 0.1% trifluoracetic acid (A) and acetonitrile (B) with a gradient of 95% A and 5% B to 20% A and 80% B over a period of 15 min at a flow rate of 1.0 mL/min. The radioligands were diluted in Milli-Q water containing Nα-DTPA (50 μM) prior to injection into HPLC. FIG. 5 shows representative HPLC chromatograms.

### Example 5: n-Octanol/PBS Distribution Coefficient

The n-octanol/PBS distribution coefficient of the five exemplified PSMA-binding agents ¹⁷⁷Lu-Ibu-PSMA, ¹⁷⁷Lu-Ibu-Dα-PSMA, ¹⁷⁷Lu-Ibu-Dβ-PSMA, ¹⁷⁷Lu-Ibu-N-PSMA and ¹⁷⁷Lu-Ibu-DAB-PSMA in a n-octanol/PBS system was performed in a similar manner as previously reported (Benesova, M.; Umbricht, C. A.; Schibli, R.; Müller, C. Albumin-binding PSMA ligands: optimization of the tissue distribution profile. Mol Pharm 2018, 15, (3), 934-946).

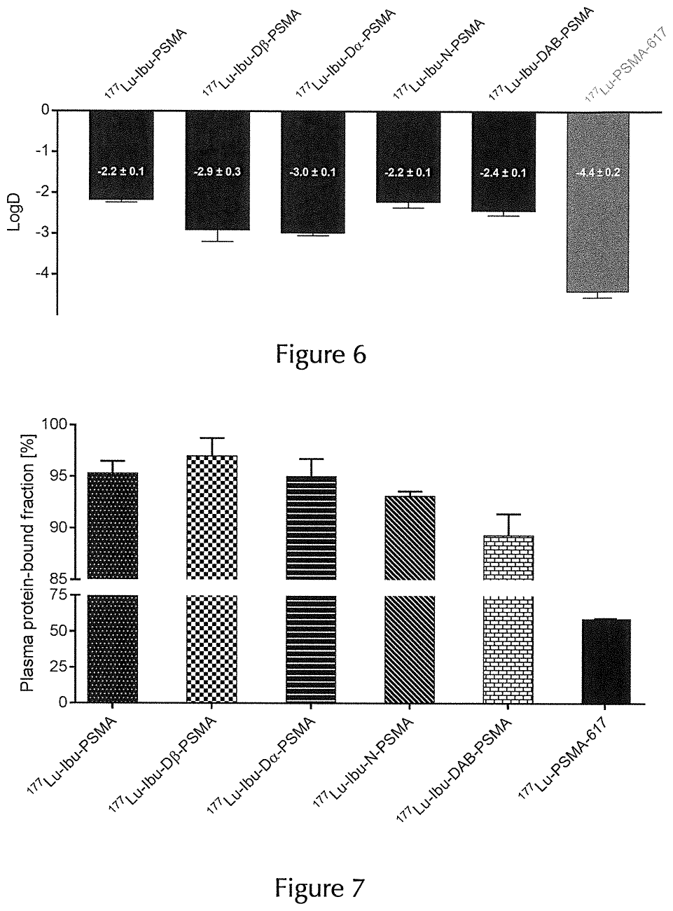

Results are shown in FIG. 6. All radioligands showed hydrophilic properties with log D values <2.2. ¹⁷⁷Lu-Ibu-PSMA, ¹⁷⁷Lu-Ibu-N-PSMA and ¹⁷⁷Lu-Ibu-DAB-PSMA showed similar values, while the coefficients of ¹⁷⁷Lu-Ibu-Dβ-PSMA and ¹⁷⁷Lu-Ibu-Dα-PSMA were slightly lower, indicating more hydrophilic properties. The modification of the PSMA ligands with ibuprofen had an effect towards more hydrophobic properties of the radioligands as compared to prior art PSMA-ligand ¹⁷⁷Lu-PSMA-617, which does not contain an albumin-binding entity.

### Example 6: In Vitro Albumin-Binding Properties

Plasma protein-binding properties of the five exemplified PSMA-binding agents ¹⁷⁷Lu-Ibu-PSMA, ¹⁷⁷Lu-Ibu-Dα-PSMA, ¹⁷⁷Lu-Ibu-Dβ-PSMA, ¹⁷⁷Lu-Ibu-N-PSMA and ¹⁷⁷Lu-Ibu-DAB-PSMA as well as of prior art PSMA-binding agent ¹⁷⁷Lu-PSMA-617 (which does not contain an albumin-binding entity) was determined using an ultrafiltration assay in a similar manner as previously reported (Benesova, M.; Umbricht, C. A.; Schibli, R.; Müller, C. Albumin-binding PSMA ligands: optimization of the tissue distribution profile. Mol Pharm 2018, 15, (3), 934-946). In short, the PSMA-ligands were labeled with ¹⁷⁷Lu at a specific activity of 50 MBq/nmol and incubated in human plasma samples or PBS at room temperature. The free and plasma-bound fractions were separated using a centrifree ultrafiltration device (4104 centrifugal filter units; Millipore, 30000 Da nominal molecular weight limit, methylcellulose micropartition membranes). The incubated solution was loaded to the ultrafiltration device and centrifuged at 2500 rpm for 40 min at 20° C. Samples from the filtrate were taken and analyzed for radioactivity in a γ-counter. The amount of plasma-bound radioligand was calculated as the fraction of radioactivity measured in the filtrate relative to the corresponding loading solution (set to 100%). The experiments were performed at least 3 times for each radioligand.

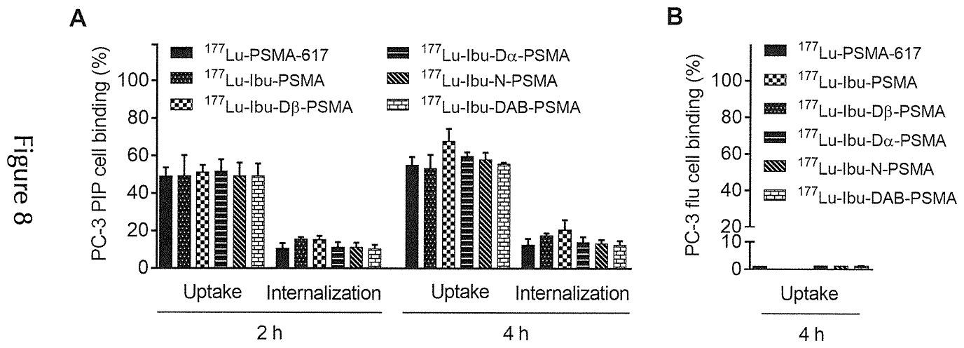

Results are shown in FIG. 7. The ultrafiltration experiments of ¹⁷⁷Lu-Ibu-PSMA, ¹⁷⁷Lu-Ibu-Dα-PSMA, ¹⁷⁷Lu-Ibu-Dβ-PSMA, ¹⁷⁷Lu-Ibu-N-PSMA and ¹⁷⁷Lu-Ibu-DAB-PSMA revealed high serum protein binding, visible by the fact that <11% of the radioligands penetrated the filter membrane when incubated in human plasma. The radioligands did not show any retention by the filter membrane when incubated in PBS (which does not contain proteins). ¹⁷⁷Lu-Ibu-N-PSMA and ¹⁷⁷Lu-Ibu-DAB-PSMA showed slightly reduced plasma protein-binding properties as compared to the other ibuprofen-derivatized radioligands. All five exemplified PSMA-binding agents ¹⁷⁷Lu-PSMA-ligands showed increased binding to plasma proteins when compared to ¹⁷⁷Lu-PSMA-617 which showed an albumin-bound fraction of only about 59%.

### Example 7: In Vitro Cell Internalization Study

Cell uptake and internalization of ¹⁷⁷Lu-Ibu-PSMA, ¹⁷⁷Lu-Ibu-Dα-PSMA, ¹⁷⁷Lu-Ibu-Dβ-PSMA, ¹⁷⁷Lu-Ibu-N-PSMA and ¹⁷⁷Lu-Ibu-DAB-PSMA were investigated using PSMA-positive PC-3 PIP and PSMA-negative PC-3 flu tumor cells kindly provided by Prof. Dr. Martin Pomper (John Hopkins Institutions, Baltimore, U.S.; Eiber, M.; Fendler, W. P.; Rowe, S. P.; Calais, J.; Hofman, M. S.; Maurer, T.; Schwarzenboeck, S. M.; Kratowchil, C.; Herrmann, K.; Giesel, F. L. Prostate-specific membrane antigen ligands for imaging and therapy. J Nucl Med 2017, 58, (Suppl 2), 67S-76S). Each radioligand was investigated by the performance of experiments performed 3 times in triplicate with PC-3 PIP tumor cell and once in triplicate with PC-3 flu tumor cells.

Results are shown in FIG. 8. The uptake of all radioligands into PC-3 PIP tumor cells was comparable to ¹⁷⁷Lu-PSMA-617 after incubation of 2 h or 4 h, respectively (FIG. 8A). The internalized fraction of ¹⁷⁷Lu-Ibu-PSMA and ¹⁷⁷Lu-Dβ-PSMA was slightly higher than for ¹⁷⁷Lu-Ibu-Dα-PSMA, ¹⁷⁷Lu-Ibu-N-PSMA, ¹⁷⁷Lu-Ibu-DAB-PSMA and ¹⁷⁷Lu-PSMA-617, which were all in the same range (FIG. 8A). The uptake of all radioligands in PC-3 flu tumor cells was <2% after 4 h, which indicated a highly PSMA-specific cell uptake (FIG. 8B).

### Example 8: In Vivo Biodistribution Study

In vivo experiments were approved by the local veterinarian department and conducted in accordance with the Swiss law of animal protection. Mice were obtained from Charles River Laboratories, Sulzfeld, Germany, at the age of 5-6 weeks. Female, athymic nude Balb/c mice were subcutaneously inoculated with PSMA-positive PC-3 PIP cells (6×10⁶ cells in 100 μL Hank's balanced salt solution (HBSS) with Ca²⁺/Mg²⁺) on the right shoulder and with PSMA-negative PC-3 flu cells (5×10⁶ cells in 100 μL HBSS Ca²⁺/Mg²⁺) on the left shoulder. Two weeks later, the tumors reached a size of about 80-300 mm³ suitable for the performance of the biodistribution studies.

Biodistribution studies were performed 12-15 days after PC-3 PIP/flu tumor cell inoculation. The radioligands ¹⁷⁷Lu-Ibu-PSMA, ¹⁷⁷Lu-Ibu-Dβ-PSMA, ¹⁷⁷Lu-Ibu-Dα-PSMA, ¹⁷⁷Lu-Ibu-N-PSMA, ¹⁷⁷Lu-Ibu-DAB-PSMA and ¹⁷⁷Lu-PSMA-617 were diluted in 0.9% NaCl containing 0.05% bovine serum albumin (BSA) to prevent adhesion to the vial and syringe material. The radioligands were injected in a lateral tail vein in a volume of 100-200 μL. Mice were euthanized at different time points after injection (p.i.) of the radioligands. Selected tissues and organs were collected, weighed and measured using a γ-counter. The results were decay-corrected and listed as a percentage of the injected activity per gram of tissue mass (% IA/g) (Table 3 and 4).

TABLE 3
Biodistribution data of ¹⁷⁷Lu-Ibu-PSMA, ¹⁷⁷Lu-Ibu-Dβ-PSMA
and ¹⁷⁷Lu-Ibu-Dα-PSMA in PC-3 PIP/flu tumor-bearing mice.
Average value ± SD obtained from each group of mice (n = 3-6).

| | ¹⁷⁷Lu-Ibu-PSMA | | ¹⁷⁷Lu-Ibu-Dβ-PSMA | | ¹⁷⁷Lu-Ibu-Dα-PSMA | |
|---|---|---|---|---|---|---|
| | 4 h.p.i. | 24 h.p.i. | 4 h.p.i. | 24 h.p.i. | 4 h.p.i. | 24 h.p.i. |
| Blood | 5.96 ± 1.53 | 0.58 ± 0.09 | 13.2 ± 1.15 | 1.28 ± 0.05 | 2.33 ± 0.71 | 0.33 ± 0.06 |
| Heart | 2.28 ± 0.68 | 0.33 ± 0.06 | 4.19 ± 0.23 | 0.57 ± 0.02 | 0.89 ± 0.27 | 0.18 ± 0.03 |
| Lung | 3.72 ± 0.73 | 0.62 ± 0.12 | 8.55 ± 3.30 | 1.17 ± 0.06 | 1.54 ± 0.35 | 0.39 ± 0.09 |
| Spleen | 1.76 ± 0.23 | 0.68 ± 0.15 | 2.60 ± 0.23 | 1.03 ± 0.08 | 0.90 ± 0.04 | 0.46 ± 0.10 |
| Kidneys | 32.5 ± 0.86 | 16.5 ± 1.48 | 32.2 ± 2.46 | 21.3 ± 1.53 | 27.2 ± 3.31 | 18.2 ± 3.06 |
| Stomach | 0.79 ± 0.26 | 0.23 ± 0.02 | 1.51 ± 0.18 | 0.48 ± 0.13 | 0.25 ± 0.08 | 0.12 ± 0.03 |
| Intestines | 1.00 ± 0.27 | 0.23 ± 0.04 | 1.81 ± 0.23 | 0.40 ± 0.07 | 0.49 ± 0.08 | 0.11 ± 0.02 |
| Liver | 2.77 ± 0.43 | 0.91 ± 0.12 | 3.87 ± 0.16 | 0.69 ± 0.07 | 0.84 ± 0.22 | 0.37 ± 0.04 |
| Salivary | 1.66 ± 0.36 | 0.34 ± 0.07 | 3.33 ± 0.15 | 0.61 ± 0.02 | 0.74 ± 0.20 | 0.21 ± 0.04 |
| glands | | | | | | |
| Muscle | 0.97 ± 0.37 | 0.12 ± 0.04 | 1.79 ± 0.48 | 0.22 ± 0.04 | 0.35 ± 0.08 | 0.07 ± 0.02 |
| Bone | 1.01 ± 0.18 | 0.20 ± 0.04 | 1.87 ± 0.16 | 0.26 ± 0.03 | 0.37 ± 0.10 | 0.11 ± 0.02 |
| PC-3 PIP | 81.3 ± 6.28 | 86.9 ± 18.0 | 65.7 ± 7.31 | 106 ± 9.70 | 49.4 ± 5.33 | 84.2 ± 14.9 |
| Tumor | | | | | | |
| PC-3 flu | 2.19 ± 0.52 | 0.58 ± 0.19 | 3.86 ± 0.18 | 0.79 ± 0.12 | 1.00 ± 0.21 | 0.38 ± 0.05 |
| Tumor | | | | | | |
| Tumor-to- | 14.1 ± 2.25 | 149 ± 16.7 | 5.03 ± 0.73 | 83.6 ± 9.06 | 22.5 ± 5.28 | 198 ± 32.6 |
| blood | | | | | | |
| Tumor-to- | 29.7 ± 3.11 | 95.4 ± 10.6 | 17.0 ± 1.73 | 151 ± 3.37 | 60.8 ± 11.1 | 196 ± 44.4 |
| liver | | | | | | |
| Tumor-to- | 2.60 ± 0.08 | 5.36 ± 0.73 | 2.06 ± 0.23 | 4.90 ± 0.29 | 1.82 ± 0.03 | 3.56 ± 0.34 |
| kidney | | | | | | |

TABLE 4
Biodistribution of ¹⁷⁷Lu-Ibu-N-PSMA, ¹⁷⁷Lu-Ibu-DAB-PSMA and
¹⁷⁷Lu-PSMA-617 in PC-3 PIP/flu tumor-bearing mice. Average
value ± SD obtained from each group of mice (n = 3-6).

| | ¹⁷⁷Lu-Ibu-N-PSMA | | ¹⁷⁷Lu-Ibu-DAB-PSMA | | ¹⁷⁷Lu-PSMA-617 | |
|---|---|---|---|---|---|---|
| | 4 h.p.i. | 24 h.p.i. | 4 h.p.i. | 24 h.p.i. | 4 h.p.i. | 24 h.p.i. |
| Blood | 3.58 ± 1.39 | 0.25 ± 0.07 | 3.66 ± 0.45 | 0.16 ± 0.02 | 0.02 ± 0.00 | 0.01 ± 0.00 |
| Heart | 1.23 ± 0.61 | 0.12 ± 0.03 | 1.32 ± 0.10 | 0.10 ± 0.01 | 0.03 ± 0.00 | 0.01 ± 0.00 |
| Lung | 2.20 ± 0.95 | 0.24 ± 0.06 | 2.40 ± 0.31 | 0.21 ± 0.03 | 0.07 ± 0.01 | 0.03 ± 0.00 |
| Spleen | 1.40 ± 0.75 | 0.24 ± 0.06 | 1.14 ± 0.11 | 0.32 ± 0.03 | 0.15 ± 0.04 | 0.05 ± 0.01 |
| Kidneys | 27.1 ± 8.37 | 8.02 ± 1.13 | 19.4 ± 1.84 | 6.00 ± 0.68 | 3.68 ± 1.05 | 0.76 ± 0.15 |
| Stomach | 0.51 ± 0.23 | 0.17 ± 0.09 | 0.62 ± 0.12 | 0.18 ± 0.13 | 0.08 ± 0.03 | 0.03 ± 0.01 |
| Intestines | 0.50 ± 0.18 | 0.09 ± 0.02 | 0.71 ± 0.05 | 0.11 ± 0.03 | 0.07 ± 0.05 | 0.04 ± 0.01 |
| Liver | 1.27 ± 0.55 | 0.32 ± 0.05 | 1.50 ± 0.12 | 0.56 ± 0.10 | 0.09 ± 0.01 | 0.07 ± 0.02 |
| Salivary | 0.94 ± 0.38 | 0.57 ± 0.35 | 0.86 ± 0.38 | 0.56 ± 0.37 | 0.04 ± 0.01 | 0.02 ± 0.00 |
| glands | | | | | | |
| Muscle | 0.47 ± 0.19 | 0.06 ± 0.02 | 0.54 ± 0.09 | 0.05 ± 0.01 | 0.02 ± 0.00 | 0.01 ± 0.00 |
| Bone | 0.56 ± 0.22 | 0.08 ± 0.02 | 0.62 ± 0.08 | 0.09 ± 0.02 | 0.06 ± 0.02 | 0.03 ± 0.01 |
| PC-3 PIP | 65.4 ± 15.6 | 58.0 ± 21.0 | 66.2 ± 11.0 | 52.4 ± 2.35 | 56.0 ± 7.95 | 37.3 ± 5.80 |
| Tumor | | | | | | |
| PC-3 flu | 1.15 ± 0.52 | 0.23 ± 0.01 | 1.19 ± 0.30 | 0.17 ± 0.01 | 0.08 ± 0.01 | 0.05 ± 0.01 |
| Tumor | | | | | | |
| Tumor-to- | 20.1 ± 6.22 | 227 ± 41.7 | 18.2 ± 2.69 | 337 ± 40.4 | 2315 ± 132 | 2730 ± 239 |
| blood | | | | | | |
| Tumor-to- | 18.2 ± 2.70 | 182 ± 52.6 | 44.6 ± 8.41 | 98.3 ± 21.1 | 598 ± 33.2 | 528 ± 62.4 |
| liver | | | | | | |
| Tumor-to- | 2.48 ± 0.11 | 6.90 ± 2.43 | 3.01 ± 0.49 | 8.88 ± 0.99 | 15.7 ± 2.79 | 49.5 ± 4.48 |
| kidney | | | | | | |

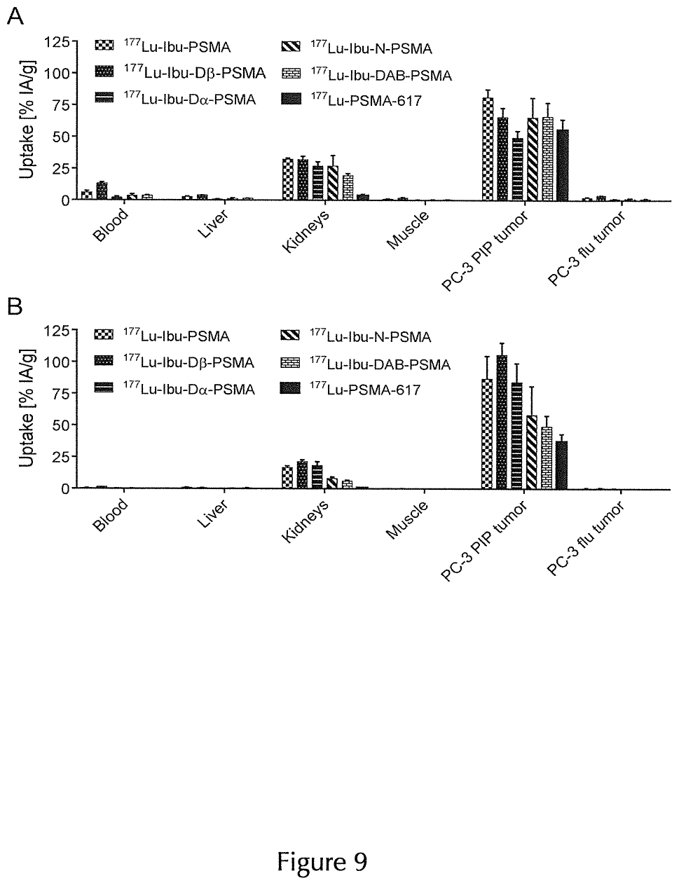

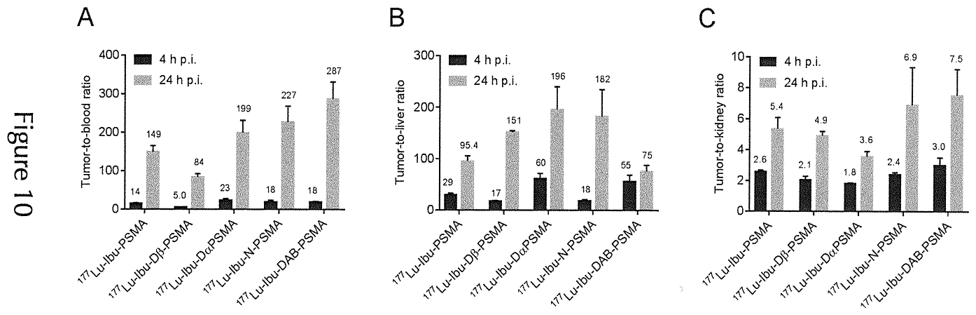

The biodistribution data and the tumor-to-background ratios are also shown in FIGS. 9 and 10, respectively.

Uptake into the PC-3 PIP tumors was fastest for ¹⁷⁷Lu-Ibu-PSMA, which was designed without an additional amino acid-based spacer entity. The tumor accumulation reached 81.3±6.28% IA/g already at 4 h p.i. and was even slightly higher at 24 h p.i. (86.8±18.0% IA/g). ¹⁷⁷Lu-Ibu-Dβ-PSMA, ¹⁷⁷Lu-Ibu-N-PSMA and ¹⁷⁷Lu-Ibu-DAB-PSMA demonstrated similar accumulation in PC-3 PIP tumors at 4 h p.i., respectively (65-66% IA/g), but different retention in the tumor tissue. At 24 h p.i., a strongly increased tumor uptake was found for ¹⁷⁷Lu-Ibu-Dβ-PSMA (106±9.70% IA/g), while radioactivity levels decreased in the case of ¹⁷⁷Lu-Ibu-N-PSMA and ¹⁷⁷Lu-Ibu-DAB-PSMA (52-58% IA/g). In the case of ¹⁷⁷Lu-Ibu-Dα-PSMA, high accumulation in the tumor was only found after 24 h (84.2±14.9% IA/g). The tumor uptake of all radioligands containing ibuprofen was higher than after injection of prior art radioligand ¹⁷⁷Lu-PSMA-617 (37.3±5.80% IA/g) at 24 h p.i. Uptake in PC-3 flu tumors (PSMA-negative) was clearly below blood levels after injection of all radioligands confirming the PSMA-mediated uptake.

Highest blood activity levels (13.2±1.15% IA/g) were detected for ¹⁷⁷Lu-Ibu-Dβ-PSMA at 4 h p.i., while all other compounds showed lower radioactivity accumulation in the blood pool at this time point (2.33-5.96% IA/g). Mice injected with ¹⁷⁷Lu-Ibu-PSMA, ¹⁷⁷Lu-Lbu-Dα-PSMA, ¹⁷⁷Lu-Ibu-N-PSMA and ¹⁷⁷Lu-Ibu-DAB-PSMA showed fast clearance of radioactivity from the blood resulting in <0.6% IA/g after 24 h whereas clearance of ¹⁷⁷Lu-Ibu-Dβ-PSMA was slower resulting in still ˜1.3% IA/g at this same time point.

Kidney uptake was lowest for ¹⁷⁷Lu-Ibu-DAB-PSMA at both 4 h and 24 h p.i. (19.4±1.84% and 6.00±0.68% IA/g, respectively) whereas the other radioligands showed a kidney uptake of 27-33% IA/g at 4 h p.i. ¹⁷⁷Lu-Ibu-N-PSMA demonstrated fastest renal clearance resulting in 8.02±1.13% IA/g at 24 h p.i. reaching a similar level as ¹⁷⁷Lu-Ibu-DAB-PSMA. Radioactivity levels in all other tissues were below the blood levels and decreased overtime.

Tumor-to-blood ratios of accumulated radioactivity were similar after injection of ¹⁷⁷Lu-Ibu-PSMA, ¹⁷⁷Lu-Ibu-Dα-PSMA, ¹⁷⁷Lu-Ibu-N-PSMA and ¹⁷⁷Lu-Ibu-DAB-PSMA (14-23), but lower after injection of ¹⁷⁷Lu-Ibu-Dβ-PSMA (5.03±0.73) at 4 h p.i. At 24 h p.i., the tumor-to-blood ratio of ¹⁷⁷Lu-Ibu-DAB-PSMA (˜337) was highest, followed by ¹⁷⁷Lu-Ibu-N-PSMA (˜227), ¹⁷⁷Lu-Ibu-Dα-PSMA (˜198), ¹⁷⁷Lu-Ibu-PSMA (˜149) and ¹⁷⁷Lu-Ibu-Dβ-PSMA (˜84). Tumor-to-kidney ratios were similar for all radioligands at 4 h p.i., but differed by a factor of ˜2 at 24 h p.i. with the highest ratios obtained after injection of ¹⁷⁷Lu-Ibu-DAB-PSMA and ¹⁷⁷Lu-Ibu-N-PSMA. The tumor-to-liver ratio at 24 h p.i. was highest for ¹⁷⁷Lu-Ibu-Dα-PSMA (196) and ¹⁷⁷Lu-Ibu-N-PSMA (182).

### Example 9: In Vivo Whole-Body-Activity Measurements

In vivo experiments were approved by the local veterinarian department and conducted in accordance with the Swiss law of animal protection. Mice were obtained from Charles River Laboratories, Sulzfeld, Germany, at the age of 5-6 weeks. Female, athymic nude Balb/c mice were subcutaneously inoculated with PSMA-positive PC-3 PIP cells (6×10⁶ cells in 100 μL Hank's balanced salt solution (HBSS) with Ca²⁺/Mg²⁺) on the right shoulder and with PSMA-negative PC-3 flu cells (5×10⁶ cells in 100 μL HBSS Ca²⁺/Mg²⁺) on the left shoulder. Two weeks later, the tumors reached a size of about 80-300 mm³ suitable for the performance of the imaging studies.

The single radioligands (specific activity: 30 MBq/nmol) were diluted in 0.9% NaCl containing 0.05% bovine serum albumin (BSA) and i.v. injected into PC-3 PIP/flu tumor bearing mice (30 MBq, 1 nmol, 100 μL) for SPECT/CT imaging purposes. The mice were measured in a dose calibrator at 4 h, 24 h, 48 h and 72 h p.i., respectively.

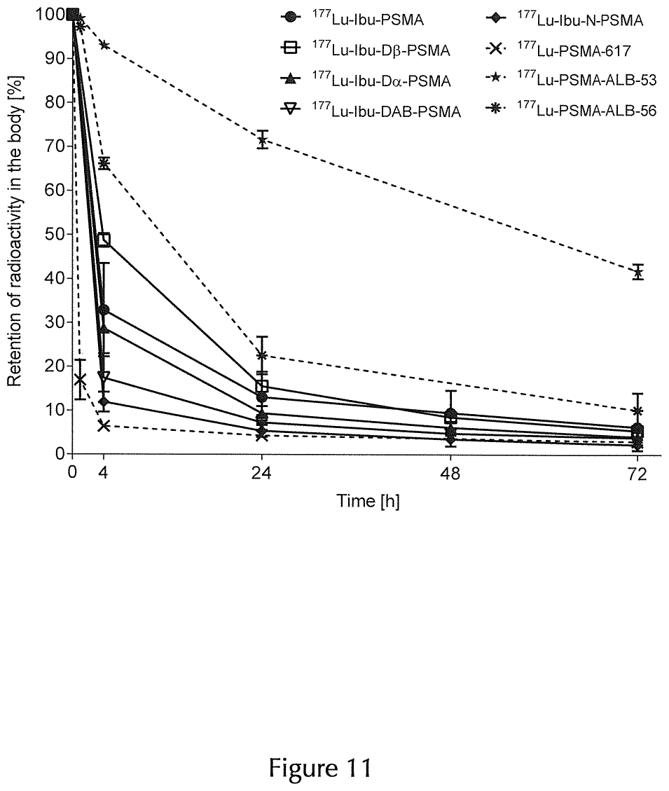

Results are shown in FIG. 11. The whole-body measurements revealed different excretion patterns for the single radioligands which was manifest most prominently at the 4 h p.i-time point. The body retention at 4 h p.i. was highest for ¹⁷⁷Lu-Ibu-Dβ-PSMA (49%) and lower for ¹⁷⁷Lu-Ibu-PSMA (33%), ¹⁷⁷Lu-Ibu-Dα-PSMA (29%) and ¹⁷⁷Lu-Ibu-DAB-PSMA (17%) with ¹⁷⁷Lu-Ibu-N-PSMA (12%) showing the lowest body retention of radioactivity. All radioligands showed higher retention of radioactivity compared to ¹⁷⁷Lu-PSMA-617 (6.5%) with limited albumin-binding properties. Activity retention was, however, reduced in comparison to comparative radioligands, ¹⁷⁷Lu-PSMA-ALB-53 (93%) and ¹⁷⁷Lu-PSMA-ALB-56 (66%), which are equipped with albumin binders based on a p-iodophenyl- and p-tolyl-entity instead of ibuprofen, respectively (Umbricht, C. A.; Benesova, M.; Schibli, R.; Müller, C. Preclinical development of novel PSMA-targeting radioligands: modulation of albumin-binding properties to improve prostate cancer therapy. Mol Pharm 2018, Mol Pharm 2018, 15, (6), 2297-2306). In all cases of ibuprofen-derivatized radioligands, retention of radioactivity in the body decreased over time and reached similar retention fractions as ¹⁷⁷Lu-PSMA-617 at 72 h p.i. At this time point, ¹⁷⁷Lu-PSMA-ALB-53 and ¹⁷⁷Lu-PSMA-ALB-56 showed still 42% and 10% of the radioactivity, respectively, retained in the body.

### Example 10: In Vivo SPECT/CT Imaging

SPECT/CT images were obtained using a dedicated small-animal SPECT/CT scanner (NanoSPECT/CT™, Mediso Medical Imaging Systems, Budapest, Hungary). SPECT/CT scans of 45 min duration were performed followed by a CT of 7.5 min. During the in vivo scans, the mice were anesthetized with a mixture of isoflurane and oxygen. Reconstruction of the acquired data was performed using HiSPECT software (version 1.4.3049, Scivis GmbH, Göttingen, Germany). All images were prepared using VivoQuant post-processing software (version 2.10, inviCRO Imaging Services and Software, Boston U.S.). A Gauss post-reconstruction filter (FWHM=1.0 mm) was applied to the images, which were presented with the scale adjusted to allow visualization of the most important organs and tissues, by cutting 5% of the lower scale.

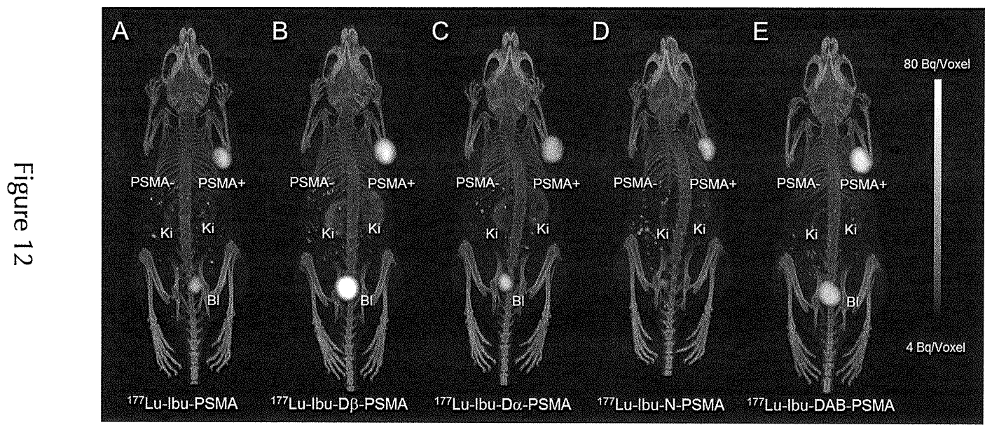

The SPECT images are shown in FIG. 12. The SPECT images visualize the PC-3 PIP tumor xenograft (right side) in which the radioligands accumulated to a high extent whereas in the PC-3 flu tumor (left side), accumulation of radioactivity was not observed. At the 4-h time point, some activity was also seen in the kidneys as well as in the urinary bladder as a consequence of renal clearance.

### Example 11: In Vitro Evaluation of ¹⁷⁷Lu-Ibu-sPSMA

In this Example, a shorter methylene linker ((CH₂)₂) than a lysine side chain ((CH₂)₄) for the spacer connection of ibuprofen was evaluated in order to examine potential effects of the spacer length on the biodistribution profile of the radioligand. For this purpose, Ibu-sPSMA (“s” for “short” spacer) was designed and synthesized (Example 8.2). Ibu-sPSMA was radiolabeled with ¹⁷⁷Lu and preclinically evaluated. The stability of ¹⁷⁷Lu-Ibu-sPSMA as well as the albumin-binding properties and the capability to bind to PSMA-positive PC-3 PIP cells were investigated. Biodistribution studies and SPECT/CT imaging studies were performed with PC-3 PIP/flu tumor bearing mice. The new data was compared with those obtained with ¹⁷⁷Lu-PSMA-617 and with ibuprofen-functionalized PSMA radioligands or ¹⁷⁷Lu-PSMA-ALB-56.

In vitro studies were conducted with ¹⁷⁷Lu-Ibu-sPSMA and compared to the results previously obtained with ¹⁷⁷Lu-PSMA-617 and, if appropriate, with ¹⁷⁷Lu-PSMA-ALB-56 (Umbricht et al, Mol Pharm 2018, 15, (6):2297-2306). Labeling efficiencies, n-octanol/PBS distribution coefficients (log D values) and albumin-binding studies were carried out. Uptake and internalization experiments were performed using the PSMA-transfected PSMA-positive PC-3 PIP tumor cell line and the mock-transfected PSMA-negative PC-3 flu tumor cell line.

### 11.1. Radiolabeling

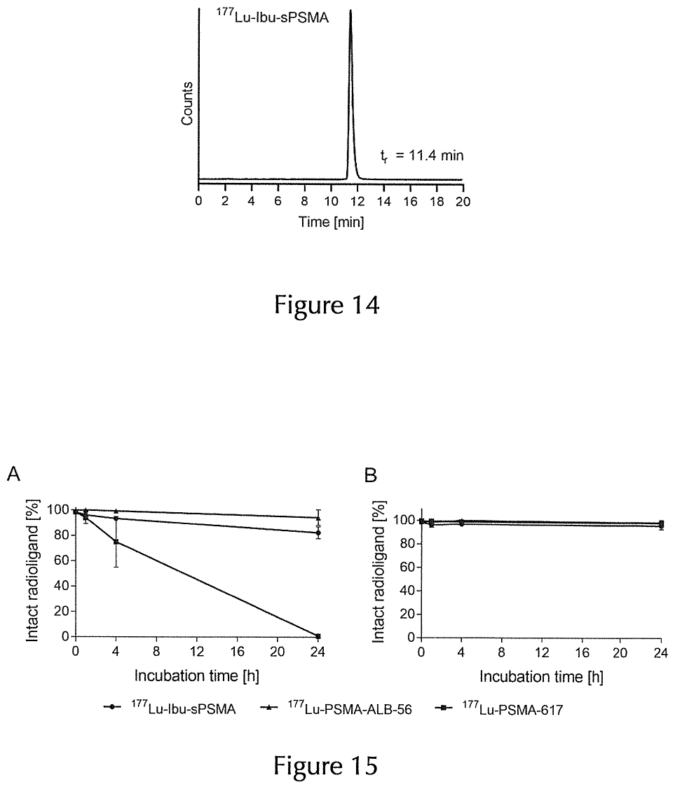

Ibu-sPSMA was diluted in Milli-Q water/DMSO in a 3:1 (v/v) mixture to obtain a final concentration of 1 mM. The Ibu-sPSMA was labeled with ¹⁷⁷Lu (no-carrier added ¹⁷⁷Lu in 0.05 M HCl; Isotope Technologies Garching ITG GmbH, Germany) in a 1:5 (v/v) mixture of sodium acetate (0.5 M, pH 8) and HCl (0.05 M, pH ˜1) at pH ˜4.5. Ibu-sPSMA was labeled with ¹⁷⁷Lu at molar activities between 5-50 MBq/nmol, depending on the experiment to be performed. The reaction mixture was incubated for 10 min at 95° C., followed by a quality control using HPLC with a C-18 reversed-phase column (Xterra™ MS, C18, 5 μm, 150×4.6 mm; Waters). The mobile phase consisted of MilliQ water containing 0.1% trifluoroacetic acid (A) and acetonitrile (B) with a gradient of 95% A and 5% B to 20% A and 80% B over a period of 15 min at a flow rate of 1.0 mL/min. The radioligands were diluted in Milli-Q water containing Nα-DTPA (50 μM) prior to injection into HPLC (FIG. 14).

### 11.2. Radiolytic Stability

Radiolytic stability over time was assessed for Ibu-sPSMA in three independent experiments. For this purpose, Ibu-sPSMA was labeled with ¹⁷⁷Lu in a volume of 120 μL at a specific activity of 50 MBq/nmol with or without the addition of L-ascorbic acid (3 mg). After quality control using HPLC (t=0, radiochemical purity ≥98%), the labeling solutions were diluted with saline to 250 MBq/500 μL and incubated at room temperature. The radioligand's integrity was determined by HPLC after 1 h, 4 h and 24 h incubation time as previously reported (Siwowska et al., Mol. Pharmaceutical 2017, 14, (2), 523-532). The HPLC chromatograms were analyzed by integration of the peaks representing the radiolabeled product, the released ¹⁷⁷Lu as well as degradation products of unknown structure (FIG. 15). A quantitative assessment was performed by expressing the peak area of the intact product as percentage of the sum of integrated peak areas of the entire chromatogram.

11.3. n-Octanol/PBS Distribution Coefficient

The n-octanol/PBS distribution coefficient of ¹⁷⁷Lu-Ibu-sPSMA was performed according to the publication by Benesova, M. et al. Mol Pharm 2018, 15, (3), 934-946). ¹⁷⁷Lu-Ibu-sPSMA revealed a value of −2.43±0.01. The modification of the PSMA ligand with ibuprofen had an effect towards more hydrophobic properties of the radioligands as compared to ¹⁷⁷Lu-PSMA-617 (−4.38±0.01). The hydrophilicity of ¹⁷⁷Lu-Ibu-sPSMA was in the same range as the other ibuprofen-derivatized ligands and ¹⁷⁷Lu-PSMA-ALB-56 (−2.9±0.2).

### 11.3. Albumin-Binding Properties

Plasma protein-binding properties of ¹⁷⁷Lu-Ibu-sPSMA were determined using an ultrafiltration assay according to Benesova, M. et al. (Mol Pharm 2018, 15, (3), 934-946). In short, the Ibu-sPSMA-ligand was labeled with ¹⁷⁷Lu at a molar activity of 50 MBq/nmol and incubated in human plasma samples or PBS at 37° C. The free and plasma-bound fraction were separated using a centrifree ultrafiltration device (4104 centrifugal filter units; Millipore, 30000 Da nominal molecular weight limit, methylcellulose micropartition membranes). The incubated solution was loaded to the ultrafiltration device and centrifuged at 2000 rpm for 40 min at 20° C. Samples from the filtrate were taken and analyzed for radioactivity in a γ-counter. The amount of plasma-bound radioligand was calculated as the fraction of radioactivity measured in the filtrate relative to the corresponding loading solution (set to 100%). The experiments were performed in triplicates.

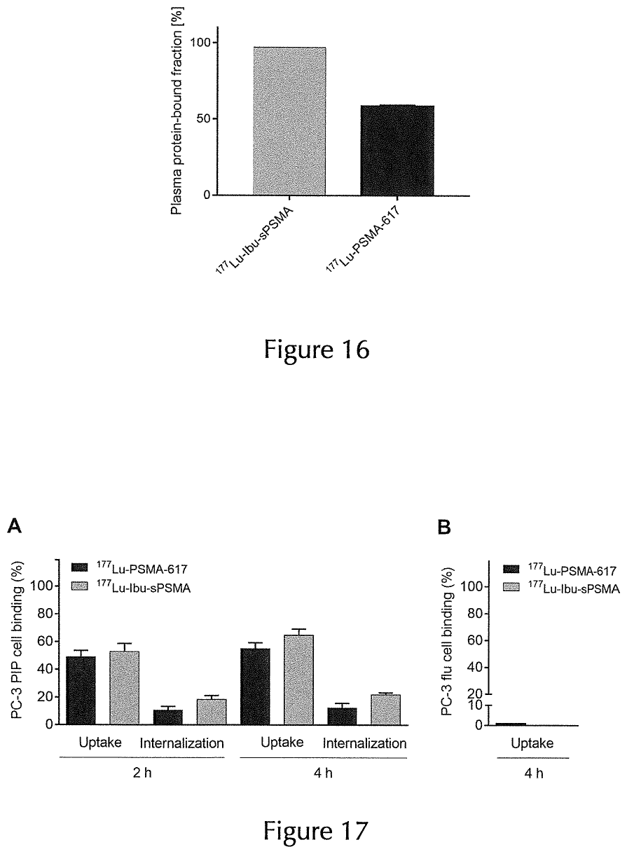

The ultrafiltration experiments of ¹⁷⁷Lu-Ibu-sPSMA revealed high serum protein binding, demonstrated by the fact that ˜97% of the radioligand were retained in the filter membrane after incubation in human plasma. The radioligand did not show any retention by the filter membrane when incubated in PBS (which does not contain proteins). ¹⁷⁷Lu-Ibu-sPSMA showed increased binding to plasma proteins when compared to ¹⁷⁷Lu-PSMA-617, which showed an albumin-bound fraction of only about 59% (FIG. 16).

### 11.4. Cell Internalization Study

Cell uptake and internalization of ¹⁷⁷Lu-Ibu-sPSMA were investigated using PSMA-positive PC-3 PIP and PSMA-negative PC-3 flu tumor cells kindly provided by Prof. Dr. Martin Pomper (Johns Hopkins University School of Medicine, Baltimore, Md., U.S.A.) (Eiber, et al.; J Nucl Med 2017, 58, (Suppl 2), 67S-76S). ¹⁷⁷Lu-Ibu-sPSMA was investigated by performing experiments 3 times in 6 replicates with PC-3 PIP tumor cells and 3 times in 6 replicates with PC-3 flu tumor cells.

The uptake and internalization of ¹⁷⁷Lu-Ibu-sPSMA into PC-3 PIP tumor cells was slightly higher than for ¹⁷⁷Lu-PSMA-617 (FIG. 17). The internalized fraction of ¹⁷⁷Lu-Ibu-sPSMA was 18% and 22% after incubation of 2 h or 4 h, respectively (FIG. 17A). The uptake of ¹⁷⁷Lu-Ibu-sPSMA in PC-3 flu tumor cells was <0.1% after 4 h, which indicated the highly PSMA-specific cell uptake in PC-3 PIP cells (FIG. 17B).

### 11.5. Determination of K_{D} Values

The K_{D} values, indicating the PSMA-binding affinity of the novel radioligand, were determined. The K_{D} value of ¹⁷⁷Lu-Ibu-sPSMA was in the same range as the other ibuprofen-derivatized PSMA radioligands and also not substantially different from K_{D} values of ¹⁷⁷Lu-PSMA-ALB-56 and ¹⁷⁷Lu-PSMA-617, determined under the same experimental conditions (Table 5).

TABLE 5
K_{D} data of the PSMA radioligands.

| | ¹⁷⁷Lu-Ibu-sPSMA | ¹⁷⁷Lu-PSMA-ALB-56 | ¹⁷⁷Lu-PSMA-617 |
|---|---|---|---|
| K_{D} [nM] | 40 ± 5 | 30 ± 6 | 13 ± 1 |

### Example 12: In Vivo Evaluation

¹⁷⁷Lu-Ibu-sPSMA was characterized in vivo and the data were compared to those obtained with ¹⁷⁷Lu-PSMA-617 and ¹⁷⁷Lu-PSMA-ALB-56.

### 12.1. Tumor Mouse Model

Mice were obtained from Charles River Laboratories, Sulzfeld, Germany, at the age of 5-6 weeks. Female, athymic nude BALB/c mice were subcutaneously inoculated with PSMA-positive PC-3 PIP cells (6×10⁶ cells in 100 μL Hank's balanced salt solution (HBSS)) on the right shoulder and with PSMA-negative PC-3 flu cells (5×10⁶ cells in 100 μL HBSS) on the left shoulder. Two weeks later, the tumors reached a size of about 80-300 mm³ suitable for the performance of the biodistribution and imaging studies.

### 12.2. Biodistribution Study

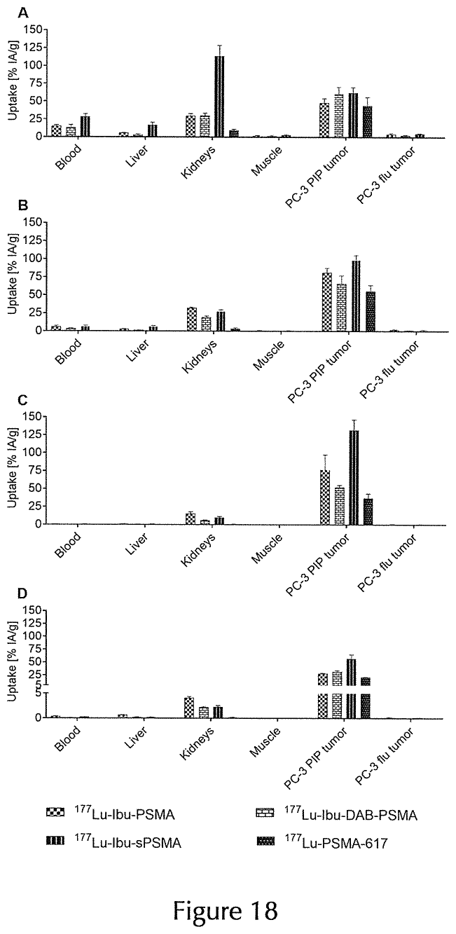

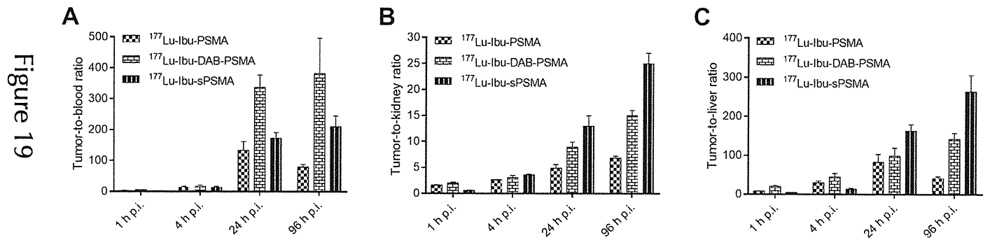

Biodistribution studies were performed 12-15 days after PC-3 PIP/flu tumor cell inoculation. ¹⁷⁷Lu-Ibu-sPSMA was diluted in 0.9% NaCl containing 0.05% bovine serum albumin (BSA) to prevent adhesion to the vial and syringe material. The radioligand was injected in a lateral tail vein in a volume of 100 μL. Mice were euthanized at different time points after injection (p.i.) of the radioligand. Selected tissues and organs were collected, weighed and measured using a γ-counter. The results were decay-corrected and listed as a percentage of the injected activity per gram of tissue mass (% IA/g) (Table 6, FIG. 18).

¹⁷⁷Lu-Ibu-sPSMA showed high accumulation in PC-3 PIP tumors already 1 h after injection (63±8% IA/g) which further increased until 24 h p.i. (132±15% IA/g) to the highest tumor uptake observed amongst all ibuprofen-bearing radioligands. Clearance of activity from tumor tissue was slow, which resulted in 57±9% IA/g retained activity in the tumor at 4 days after injection, compared to 20-34% IA/g for the other radioligands at the same time-point. Uptake into PSMA-negative PC-3 flu cells was clearly below blood levels, confirming the specific PSMA-mediated uptake in PC-3 PIP tumors.

¹⁷⁷Lu-Ibu-sPSMA showed the highest blood activity levels at 1 h p.i. (29±4% IA/g compared to 13-18% IA/g for the other ibuprofen-containing radioligands) which continuously decreased over time to similar blood activity levels as ¹⁷⁷Lu-Ibu-PSMA and ¹⁷⁷Lu-Ibu-Dα-PSMA 4 days after injection. In comparison to the blood activity levels of the fast cleared ¹⁷⁷Lu-Ibu-N-PSMA and ¹⁷⁷Lu-Ibu-DAB-PSMA, ¹⁷⁷Lu-Ibu-sPSMA exhibited approximately three times higher values at 24 h and 96 h after injection.

The uptake in the kidney was very high (114±15% IA/g) at 1 h p.i. compared to only 30-33% IA/g for ¹⁷⁷Lu-Ibu-PSMA, ¹⁷⁷Lu-Ibu-N-PSMA and ¹⁷⁷Lu-Ibu-DAB-PSMA and 73±2% IA/g for ¹⁷⁷Lu-Ibu-Dα-PSMA). Renal clearance was, however fast so that activity levels similar to the other radioligands were reached already 4 h p.i. In a similar way, the liver showed high accumulation of activity at early time-points (17±4% IA/g at 1 h p.i. and 7.2±0.5% IA/g at 4 h p.i.), but the fast clearance resulted in a similar activity retention in the liver at 24 h p.i. and 96 h p.i. compared to the other ibuprofen-bearing radioligands.

Table 6 shows biodistribution data of ¹⁷⁷Lu-Ibu-sPSMA in PC-3 PIP/flu tumor-bearing mice. The values represent the average value±SD of the percentage injected activity per gram tissue [% IA/g] obtained from each group of mice (n=4). Comparison of the features of ¹⁷⁷Lu-Ibu-sPSMA with the other ibuprofen-derivatized radioligands revealed exceptionally high accumulation and retention of activity in PSMA-positive PC-3 PIP tumors resulting in high tumor-to-kidney and tumor-to-liver ratios, in particular at late time points.

TABLE 6

| | ¹⁷⁷Lu-Ibu-sPSMA | | | |
|---|---|---|---|---|
| | 1 h.p.i. | 4 h.p.i. | 24 h.p.i. | 96 h.p.i. |
| Blood | 29 ± 4 | 7.2 ± 0.7 | 0.76 ± 0.07 | 0.27 ± 0.02 |
| Heart | 9.3 ± 1.5 | 2.4 ± 0.2 | 0.39 ± 0.04 | 0.12 ± 0.01 |
| Lung | 17 ± 3 | 4.9 ± 0.7 | 0.79 ± 0.07 | 0.25 ± 0.03 |
| Spleen | 5.8 ± 1.2 | 2.1 ± 0.3 | 0.72 ± 0.15 | 0.22 ± 0.03 |
| Kidneys | 114 ± 15 | 28 ± 2 | 10 ± 2 | 2.3 ± 0.3 |
| Stomach | 2.6 ± 0.5 | 1.3 ± 0.9 | 0.50 ± 0.19 | 0.73 ± 0.82 |
| Intestines | 2.9 ± 0.3 | 1.1 ± 0.2 | 0.31 ± 0.07 | 0.13 ± 0.03 |
| Liver | 17 ± 4 | 7.2 ± 0.5 | 0.81 ± 0.09 | 0.22 ± 0.02 |
| Salivary glands | 6.6 ± 0.5 | 1.9 ± 0.2 | 0.37 ± 0.05 | 0.11 ± 0.02 |
| Muscle | 3.1 ± 0.7 | 0.84 ± 0.14 | 0.14 ± 0.02 | 0.04 ± 0.02 |
| Bone | 3.3 ± 0.4 | 1.1 ± 0.2 | 0.27 ± 0.04 | 0.08 ± 0.01 |
| PC-3 PIP | 63 ± 8 | 99 ± 7 | 132 ± 15 | 57 ± 9 |
| Tumor | | | | |
| PC-3 flu | 5.1 ± 0.7 | 2.0 ± 0.3 | 0.66 ± 0.07 | 0.15 ± 0.02 |
| Tumor | | | | |
| Tumor-to- | 2.2 ± 0.1 | 14 ± 3 | 173 ± 18 | 210 ± 35 |
| blood | | | | |
| Tumor-to- | 3.7 ± 0.5 | 14 ± 2 | 163 ± 16 | 263 ± 42 |
| liver | | | | |
| Tumor-to- | 0.56 ± 0.09 | 3.6 ± 0.2 | 13 ± 2 | 25 ± 2 |
| kidney | | | | |

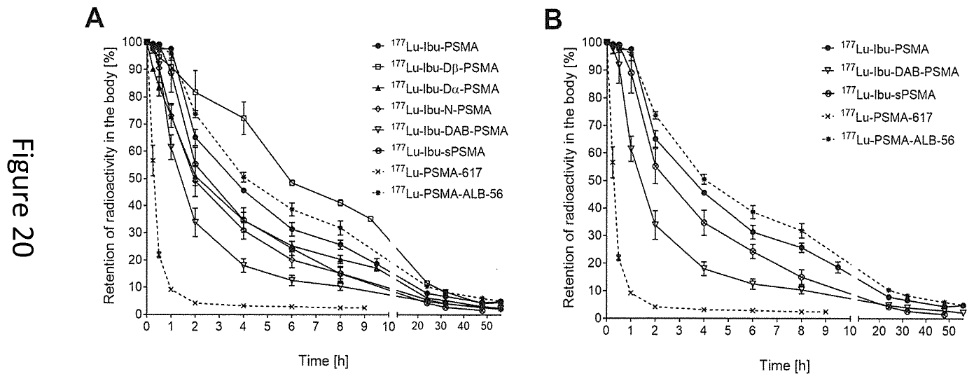

Due to the high blood activity levels, in particular at early time-points after injection, tumor-to-blood ratios of accumulated radioactivity of ¹⁷⁷Lu-Ibu-sPSMA were consistently lower as compared to ¹⁷⁷Lu-Ibu-DAB-PSMA, but reached similar values as ¹⁷⁷Lu-Ibu-PSMA, ¹⁷⁷Lu-Ibu-Dα-PSMA and ¹⁷⁷Lu-Ibu-N-PSMA at later time-points (FIG. 19A). The tumor-to-kidney ratio of ¹⁷⁷Lu-Ibu-sPSMA showed an equally low value as compared to ¹⁷⁷Lu-Ibu-Dα-PSMA (0.56±0.09 and 0.59±0.08, respectively) 1 h p.i., but increased significantly with time to give the highest ratios amongst the radioliogands at all other time-points (FIG. 19B). In a similar manner, tumor-to-liver ratios were low at 1 h and 4 h p.i., but outperformed the other radioligands at 24 h and 96 h after injection (FIG. 19C).

### 12.3. Whole-Body-Activity Measurements

All albumin-binding radioligands (molar activity: 25 MBq/nmol) were diluted in 0.9% NaCl containing 0.05% BSA and i.v. injected into non-tumor bearing mice (25 MBq, 1 nmol, 100 μL). The mice were measured in a dose calibrator at various time-points up to 56 h p.i. The radioligands were compared with previously obtained data from ¹⁷⁷Lu-PSMA-617.

The whole-body measurements revealed different excretion patterns for the single radioligands, which was manifest most prominently at early time-points up to 8 h after injection (FIG. 20). Amongst all radioligands the body retention was highest for ¹⁷⁷Lu-Ibu-Dβ-PSMA with the only exception at late time-points (48 h and 56 h p.i), where the retention of ¹⁷⁷Lu-PSMA-ALB-56, containing a p-iodophenyl entity as stronger albumin binder, was higher. The other ibuprofen-bearing radioligands showed less retention in the body as compared to ¹⁷⁷Lu-PSMA-ALB-56. Amongst the albumin-binding radioligands, ¹⁷⁷Lu-Ibu-DAB-PSMA was characterized with the fastest excretion pattern with a retained activity of only 18% already 4 h after injection in comparison to the other albumin-binding radioligands (35-73%). All radioligands showed higher retention of radioactivity compared to ¹⁷⁷Lu-PSMA-617 with limited albumin-binding properties. In all cases, retention of radioactivity in the body decreased over time and reached similar retention fractions 32 h p.i.

### 12.4. In Vivo SPECT/CT Imaging

SPECT/CT images were obtained using a dedicated small-animal SPECT/CT scanner (NanoSPECT/CT™, Mediso Medical Imaging Systems, Budapest, Hungary). SPECT/CT scans of 45 min duration were performed followed by a CT of 7.5 min. During the in vivo scans, the mice were anesthetized with a mixture of isoflurane and oxygen. Reconstruction of the acquired data was performed using HiSPECT software (version 1.4.3049, Scivis GmbH, Göttingen, Germany). All images were prepared using VivoQuant post-processing software (version 2.10, inviCRO Imaging Services and Software, Boston U.S.). A Gauss post-reconstruction filter (FWHM=1.0 mm) was applied to the images, which were presented with the scale adjusted to allow visualization of the most important organs and tissues, by cutting 5% of the lower scale.

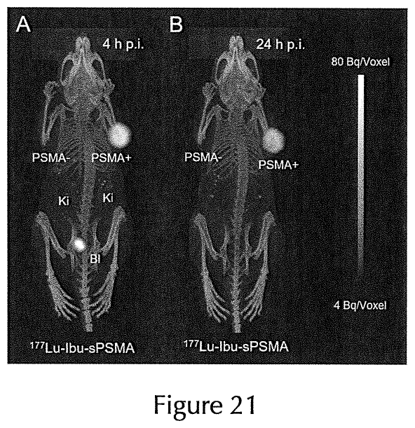

The SPECT/CT images visualized the PC-3 PIP tumor xenograft (right side of FIG. 21) in which ¹⁷⁷Lu-Ibu-sPSMA accumulated to a high extent whereas in the PC-3 flu tumor (left side of FIG. 21), accumulation of radioactivity was not observed. At the 4-h time point, some activity was also seen in the kidneys as well as in the urinary bladder as a consequence of renal clearance. At the 24-h time point, activity was only visualized in the PC-3 PIP tumor (FIG. 21).

### Example 13. In Vivo Therapy Study

The therapeutic efficacy of ¹⁷⁷Lu-Ibu-DAB-PSMA was assessed in vivo in a tumor mouse model (PSMA-positive PC-3 PIP tumor-bearing mice) and the data were compared to those obtained with ¹⁷⁷Lu-PSMA-617 and ¹⁷⁷Lu-PSMA-ALB-56 (Eiber et al., J. Nucl. Med., 2017, 58 (Suppl. 2) 67S-76S).

### 13.1. Tumor Mouse Model

Mice were obtained from Charles River Laboratories, Sulzfeld, Germany, at the age of 5-6 weeks. Female, athymic nude BALB/c mice were subcutaneously inoculated with PSMA-positive PC-3 PIP cells (4×10⁶ cells in 100 μL Hank's balanced salt solution (HBSS)) on the right shoulder. Six days later, the tumors reached a size of about 30-160 mm³ suitable for the performance of the in vivo therapy study. Mice were euthanized when a predefined endpoint criterion was reached or when the study was finalized at Day 84. Endpoint criteria were defined as (i) body weight loss of >15%, (ii) a tumor volume of >800 mm³ (iii) a combination of body weight loss of >10% and a tumor volume of >700 mm³ or (iv) signs of unease and pain or a combination thereof.

### 13.2. Methods

Six days after subcutaneous inoculation of 4×10⁶ PC-3 PIP tumor cells inoculation, three groups with statistically similar body weight and tumor volumes were intravenously injected. One group was injected with only the vehicle (saline containing 0.05% bovine serum albumin (BSA); Group A; n=6), and another two groups with ¹⁷⁷Lu-Ibu-DAB-PSMA (Group B: 2 MBq, 1 nmol (n=6) and Group C: 5 MBq, 1 nmol (n=6)) at Day 0 of the therapy study (Table 7). The monitoring of mice and the assessment of the therapy study was conducted as described by Eiber et al., J. Nucl. Med., 2017, 58 (Suppl. 2) 67S-76S). In short, the mice were monitored by measuring body weight and tumor size every second day over a period of 12 weeks. The relative body weight (RBW) was defined as [BWₓ/BW₀], where BWₓ is the body weight in grams at a given Day x and BW₀ is the body weight in grams at Day 0. The tumor dimensions were determined by measuring the longest tumor axis (L) and its perpendicular axis (W) with a digital caliper. The tumor volume (V) was calculated according to the equation [V=0.5×(LW²)]. The relative tumor volume (RTV) was defined as [TVₓ/TV₀], where TVₓ is the tumor volume in mm³ at a given day x, and TV₀ is the tumor volume in mm³ at Day 0.

TABLE 7
Design of the therapy study.

| | | | | Tumor volume^{a} | Body weight^{a} |
|---|---|---|---|---|---|
| | | | Injected | [mm³] | [g] |
| | | | radioactivity | (average ± SD) | (average ± SD) |
| Group | Treatment | n | [MBq] | Day 0 | Day 0 |
| A | Saline | 12 | — | 66 ± 30 | 17 ± 1.5 |
| B | ¹⁷⁷Lu-Ibu-DAB-PSMA | 6 | 2 | 58 ± 24 | 17 ± 1.3 |
| C | ¹⁷⁷Lu-Ibu-DAB-PSMA | 6 | 5 | 65 ± 14 | 18 ± 1.5 |
| D | ¹⁷⁷Lu-PSMA-617 | 6 | 2 | 103 ± 24 | 16 ± 1.2 |
| E | ¹⁷⁷Lu-PSMA-617 | 6 | 5 | 104 ± 25 | 17 ± 0.9 |
| F | ¹⁷⁷Lu-PSMA-ALB-56 | 6 | 2 | 81 ± 25 | 15 ± 1.3 |
| G | ¹⁷⁷Lu-PSMA-ALB-56 | 6 | 5 | 92 ± 34 | 15 ± 1.3 |

^{a}No significant differences determined between the values measured for each group (p > 0.05).

The efficacy of the radionuclide therapy was expressed as the tumor growth delay (TGDₓ), which was calculated as the time required for the tumor volume to increase x-fold over the initial volume at Day 0. The tumor growth delay index [TGDIₓ=TGDₓ(T)/TGDₓ(C)] was calculated as the TGDₓ ratio of treated mice (T) over the TGDₓ average of control mice (C) for a 5-fold (x=5, TGD₅) increase of the initial tumor volume. The median survival was calculated using GraphPad Prism software (version 7). Survival of mice was assessed using Kaplan-Meier curves to determine median survival of mice of each group using Graph Pad Prism software (version 7).

### 13.3. Results of the Therapy Study

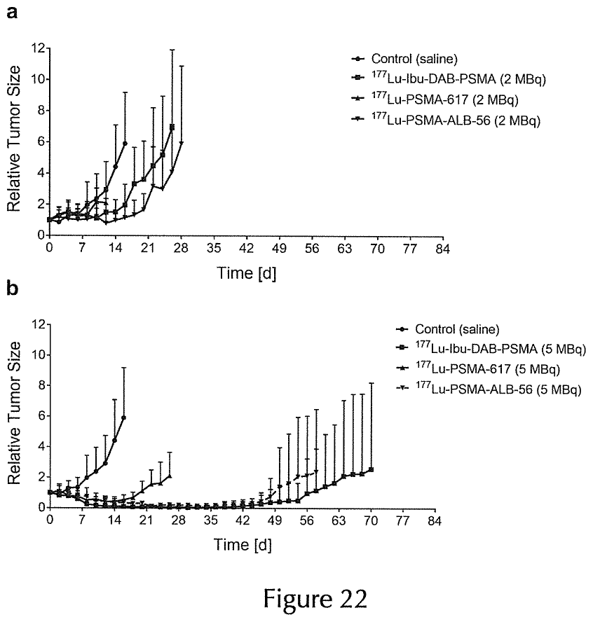

The results of the therapy study were combined with the results obtained in a therapy study including the group injected with only the vehicle (saline containing 0.05% BSA; Group A; n=6), ¹⁷⁷Lu-PSMA-617 (2 MBq and 5 MBq; Group D and E; n=6) and ¹⁷⁷Lu-PSMA-ALB-56 (2 MBq and 5 MBq; Group F and G; n=6) (Eiber et al., J. Nucl. Med., 2017, 58 (Suppl. 2) 67S-76S). The tumor growth of treated mice was more delayed than the tumor growth of untreated control mice (combined; n=12) (FIG. 22).

The tumor growth delay index five (TGDI₅) values of groups injected with 2 MBq ¹⁷⁷Lu-Ibu-DAB-PSMA (1.6) and ¹⁷⁷Lu-PSMA-ALB-56 (1.8), respectively, were clearly increased as compared to the one of the control animals (1.0 defined for controls). Only the TGDI₅ of the mice injected with 2 MBq ¹⁷⁷Lu-PSMA-617 (1.1) was comparable to the value of the control animals (Table 8).

TABLE 8
Tumor growth delay index with 5-fold increase of tumor size.

| | | first mouse | | |
|---|---|---|---|---|
| | | of group | median | |
| | | euthanized | survival | |
| Group | Treatment | [d] | [d] | TGDI₅ |
| A | Saline | 16 | 26 | 1.0 ± 0.5 |
| B | ¹⁷⁷Lu-Ibu-DAB-PSMA | 26 | 34 | 1.6 ± 0.4 |
| C | ¹⁷⁷Lu-Ibu-DAB-PSMA | 70 | n.d.^{a} | n.d.^{a} |
| D | ¹⁷⁷Lu-PSMA-617 | 12 | 19 | 1.1 ± 0.1 |
| E | ¹⁷⁷Lu-PSMA-617 | 26 | 32 | 2.0 ± 0.3 |
| F | ¹⁷⁷Lu-PSMA-ALB-56 | 28 | 36 | 1.8 ± 0.5 |
| G | ¹⁷⁷Lu-PSMA-ALB-56 | 58 | n.d.^{a} | n.d.^{a} |

^{a}n.d. = not defined since majority of mice were still alive at the end of the study.

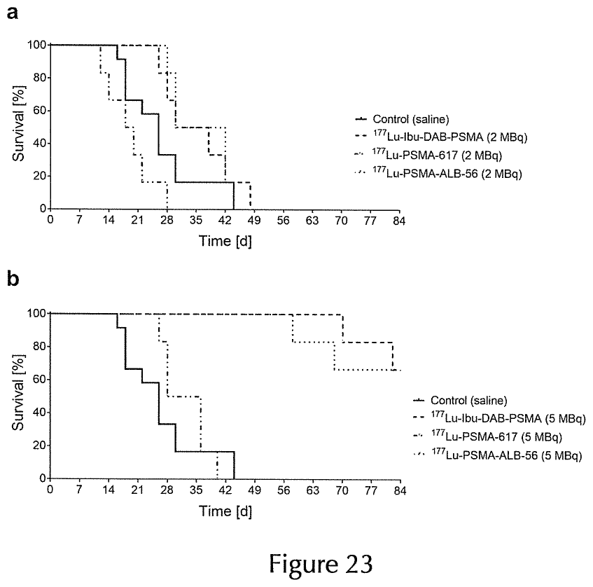

The TGDI₅ values of the groups injected with 5 MBq ¹⁷⁷Lu-PSMA-617 (2.0) was in the same range as for the albumin-binding radioligands applied at 2 MBq per mouse. The TGDI₅ values of mice injected with 5 MBq ¹⁷⁷Lu-Ibu-DAB-PSMA or ¹⁷⁷Lu-PSMA-ALB-56, respectively, were not defined as in four mice of each group the tumors disappeared entirely. Regrowth of tumors in animals with total remission was not observed until the end of the study at Day 84. In each group regrowth of the tumor was observed in two mice from about 5 weeks after therapy on so that they reached the endpoint at Day 70 and Day 82 (¹⁷⁷Lu-Ibu-DAB-PSMA) and at Day 58 and Day 68 (¹⁷⁷Lu-PSMA-ALB-56), respectively. The median survival time remained, therefore, undefined for these groups of mice which received 5 MBq ¹⁷⁷Lu-Ibu-DAB-PSMA or 5 MBq ¹⁷⁷Lu-PSMA-ALB-56, respectively (FIG. 23). At the end of the study at Day 84, four mice were still alive in each of these groups. The median survival of mice treated with 2 MBq ¹⁷⁷Lu-Ibu-DAB-PSMA and ¹⁷⁷Lu-PSMA-ALB-56, respectively, was 34 and 36 days, hence, clearly increased compared to the median survival of control mice (26 days). On the other hand, the median survival of the group injected with 2 MBq ¹⁷⁷Lu-PSMA-617 (19 days) was shorter than for all other groups including untreated control mice (FIG. 23).

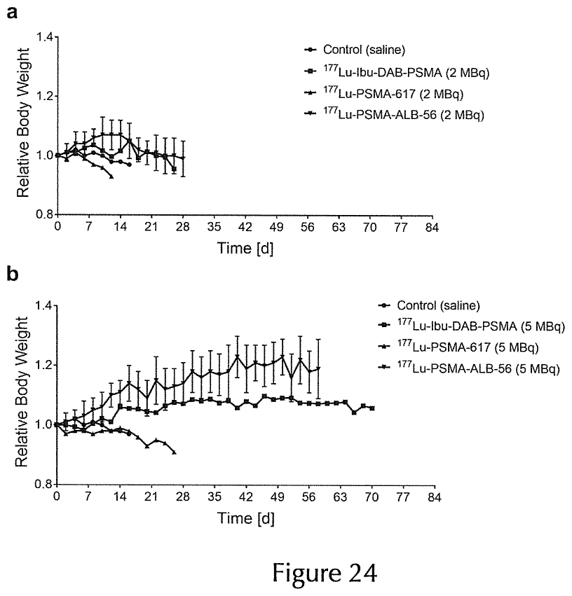

At Day 16, when the first control mouse reached the endpoint, the average relative body weight (0.93-1.10) was comparable in all groups (FIG. 24). At the time of euthanasia, the average relative body weight of the groups injected with 5 MBq ¹⁷⁷Lu-Ibu-DAB-PSMA (1.06±0.10) and ¹⁷⁷Lu-PSMA-ALB-56 (1.20±0.14), respectively, was increased as compared to the average relative body weight of control mice (0.88±0.05) and mice treated with ¹⁷⁷Lu-PSMA-617 (0.86±0.05). These findings can be ascribed to the faster tumor growth in control mice and mice, treated with ¹⁷⁷Lu-PSMA-617 and, hence, the fact that they reached the endpoint sooner than mice treated with ¹⁷⁷Lu-Ibu-DAB-PSMA or ¹⁷⁷Lu-PSMA-ALB-56 (FIG. 24).

As a result, ¹⁷⁷Lu-Ibu-DAB-PSMA performed significantly better than ¹⁷⁷Lu-PSMA-617 for both quantities of injected activity (2 MBq/mouse and 5 MBq/mouse, respectively). While ¹⁷⁷Lu-Ibu-DAB-PSMA was only slightly inferior compared to ¹⁷⁷Lu-PSMA-ALB-56 at the lower injected activity (2 MBq/mouse), it was even slightly superior when applied at the higher quantity of activity (5 MBq/mouse). The improved tumor-to-blood ratios of ¹⁷⁷Lu-Ibu-DAB-PSMA as compared to the results obtained with ¹⁷⁷Lu-PSMA-ALB-56 and the outcome of this therapy study, confirmed the superiority of ¹⁷⁷Lu-Ibu-DAB-PSMA over the existing ¹⁷⁷Lu-PSMA-617.

1. A compound according to General Formula (1)(i) or (1)(ii): 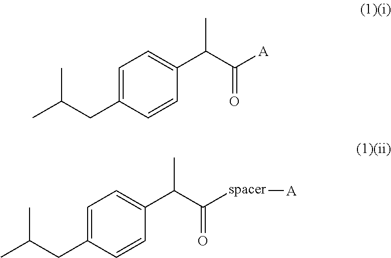
wherein A is a diagnostic or therapeutic agent comprising a binding site for a tumor antigen, and the spacer comprises at least one C—N bond.

2. The compound according to claim 1, wherein the tumor antigen is prostate-specific membrane antigen (PSMA).

3. The compound according to claim 1 or 2, wherein the diagnostic or therapeutic agent A comprises a radiolabel.

4. The compound according to claim 3, wherein the radiolabel is a non-metallic radionuclide or a radiometal.

5. The compound according to any one of claims 1-4, wherein the diagnostic or therapeutic agent A comprises a chelator.

6. The compound according to claim 5, wherein the diagnostic or therapeutic agent A comprises a radiometal coordinated via the chelator.

7. The compound according to any one of claims 1-6, wherein the compound is characterized by the following General Formula (1a): 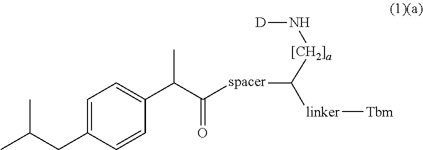
wherein D is a chelator;
Tbm is a tumor-antigen binding moiety;
linker is a linker, preferably comprising a cyclic group or an aromatic group;
spacer is a spacer comprising a C—N bond; and
a is an integer selected from 0, 1, 2, 3, 4, 5, 6, 7, 8, 9, or 10.

8. A compound characterized by the following General Formula (1a): 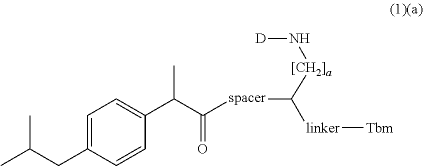
wherein D is a chelator;
Tbm is a tumor-antigen binding moiety;
linker is a linker, preferably comprising a cyclic group or an aromatic group;
spacer is a spacer comprising a C—N bond; and
a is an integer selected from 0, 1, 2, 3, 4, 5, 6, 7, 8, 9, or 10;
or a pharmaceutically acceptable salt, ester, solvate or radiolabeled complex thereof.

9. The compound according to claim 7 or 8, wherein the tumor-antigen binding moiety (Tbm) is a PSMA-binding moiety (Pbm).

10. The compound according to claim 9, wherein the PSMA-binding moiety is characterized by General Formula (3): 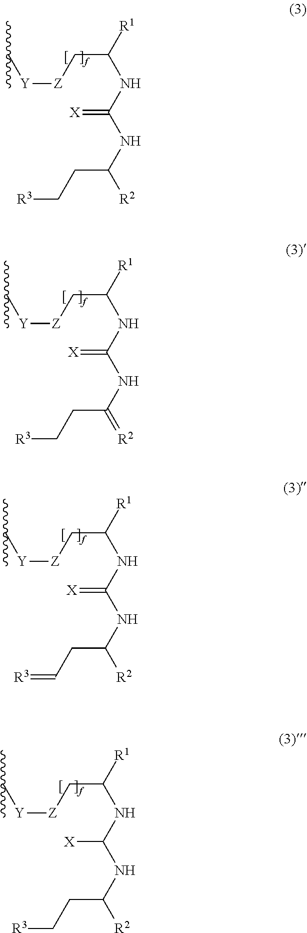
wherein
X and Y are each independently selected from O, N or NH or NH₂, S or P,
Z is selected from CH₂ or substituted CH₂, wherein one or both of the hydrogen atoms may be substituted,
R¹, R² and R³ are each independently selected from —COH, —CO₂H, —SO₂H, —SO₃H, —SO₄H, —PO₂H, —PO₃H, —PO₄H₂, —C(O)—(C₁-C₁₀)alkyl, —C(O)—O(C₁-C₁₀)alkyl, —C(O)—NHR⁴, or —C(O)—NR⁴R⁵, wherein R⁴ and R⁵ are each independently selected from H, bond, (C1-C10)alkylene, F, Cl, Br, I, C(O) or —CH(O), C(S) or —CH(S), —C(S)—NH-benzyl-, —C(O)—NH-benzyl, —C(O)—(C₁-C₁₀)alkylene, —(CH₂)ₚ—NH, —(CH₂)ₚ—(C₁-C₁₀)alkyene, —(CH₂)ₚ—NH—C(O)—(CH₂)_{q}, —(CHᵣCH₂)ₜ—NH—C(O)—(CH₂)ₚ, —(CH₂)ₚ—CO—COH, —(CH₂)ₚ—CO—CO₂H, —(CH₂)ₚ—C(O)NH—C[(CH₂)_{q}—COH]₃, —C[(CH₂)ₚ—COH]₃, —(CH₂)ₚ—C(O)NH—C[(CH₂)_{q}—CO₂H]₃, —C[(CH₂)ₚ—CO₂H]₃ or —(CH₂)ₚ—(C₅-C₁₄)heteroaryl, and
f, p, q, r and t are each independently an integer selected from 0, 1, 2, 3, 4, 5, 6, 7, 8, 9, or 10;
preferably
X O or S, and
Y NH or O or S.

11. The compound according to claim 10, wherein f is an integer selected from 1, 2, 3, 4, or 5; preferably f is 2 or 3.

12. The compound according to claim 10 or 11, wherein Y is O or NH.

13. The compound according to any one of claims 10-12, wherein Z is CH₂ or C═O.

14. The compound according to any one of claims 10-13, wherein the PSMA-binding moiety is characterized by General Formula (3)(ii): 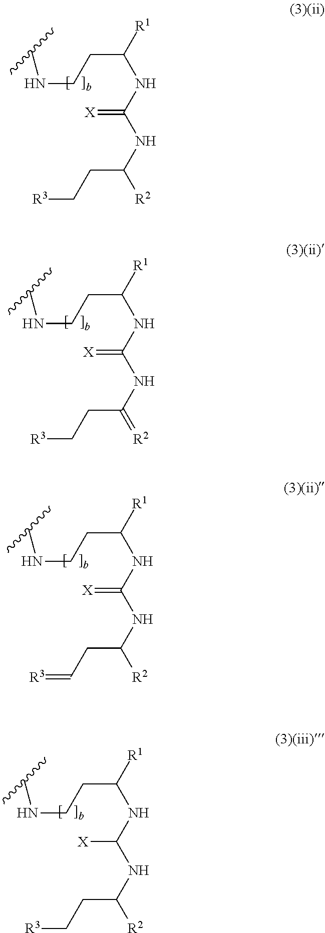
wherein
X is selected from O, N or NH or NH₂, S or P,
R¹, R² and R³ are each independently selected from —COH, —CO₂H, —SO₂H, —SO₃H, —SO₄H, —PO₂H, —PO₃H, —PO₄H₂, —C(O)—(C₁-C₁₀)alkyl, —C(O)—O(C₁-C₁₀)alkyl, —C(O)—NHR⁴, or —C(O)—NR⁴R⁵, wherein R⁴ and R⁵ are each independently selected from H, bond, (C1-C10)alkylene, F, Cl, Br, I, C(O) or —CH(O), C(S) or —CH(S), —C(S)—NH-benzyl-, —C(O)—NH-benzyl, —C(O)—(C₁-C₁₀)alkylene, —(CH₂)ₚ—NH, —(CH₂)ₚ—(C₁-C₁₀)alkyene, —(CH₂)ₚ—NH—C(O)—(CH₂)_{q}, —(CHᵣCH₂)ₜ—NH—C(O)—(CH₂)ₚ, —(CH₂)ₚ—CO—COH, —(CH₂)ₚ—CO—CO₂H, —(CH₂)ₚ—C(O)NH—C[(CH₂)_{q}—COH]₃, —C[(CH₂)ₚ—COH]₃, —(CH₂)ₚ—C(O)NH—C[(CH₂)_{q}—CO₂H]₃, —C[(CH₂)ₚ—CO₂H]₃ or —(CH₂)ₚ—(C₅-C₁₄)heteroaryl, and
b, p, q, r and t are each independently an integer selected from 0, 1, 2, 3, 4, 5, 6, 7, 8, 9, or 10;
preferably
X O or S, and
Y NH or O or S.

15. The compound according to any one of claims 10-14, wherein X is O.

16. The compound according to any one of claims 10-14, wherein R¹, R² and R³ are each independently selected from —COH, —CO₂H, —SO₂H, —SO₃H, —SO₄H, —PO₂H, —PO₃H, —PO₄H₂.

17. The compound according to claim 16, wherein each of R¹, R² and R³ is —COOH.

18. The compound according to any one of claims 14-17, wherein b is an integer selected from 1, 2, 3, 4 or 5, preferably b is 2, 3 or 4, more preferably b is 3.

19. The compound according to any one of claims 14-18, wherein R¹, R² and R³ are each COOH, X is O, and b is 3.

20. The compound according to any one of claims 9-19, wherein the PSMA-binding moiety is characterized by Formula (3)(a):

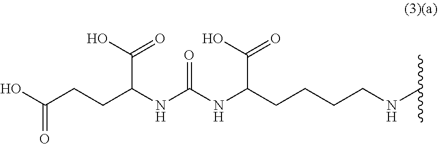 [SMILES: *NCCCCC(NC(=O)NC(CCC(=O)O)C(=O)O)C(=O)O]

21. The compound according to any one of claims 9-13, wherein the PSMA-binding moiety is characterized by Formula (3)(b):

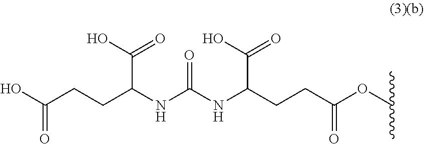 [SMILES: *OC(=O)CCC(NC(=O)NC(CCC(=O)O)C(=O)O)C(=O)O]

22. The compound according to any one of claims 7-21, wherein the linker is characterized by the Structural Formula (4): 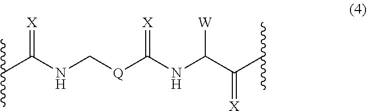
wherein
X is each independently selected from O, N, S or P,
Q is selected from substituted or unsubstituted alkyl, alkylaryl and cycloalkyl, preferably from substituted or unsubstituted C₅-C₁₄ aryl, C₅-C₁₄ alkylaryl or C₅-C₁₄ cycloalkyl, and
W is selected from —(CH₂)_{c}-aryl or —(CH₂)_{c}-heteroaryl, wherein c is an integer selected from 0, 1, 2, 3, 4, 5, 6, 7, 8, 9 or 10.

23. The compound according to claim 22, wherein each X is O.

24. The compound according to claim 22 or 23, wherein Q is selected from substituted or unsubstituted C₅-C₇ cycloalkyl.

25. The compound according to claim 24, wherein Q is cyclohexyl.

26. The compound according to any one of claims 22-25, wherein W is selected from —(CH₂)_{c}-naphthyl, —(CH₂)_{c}-phenyl, —(CH₂)_{c}-biphenyl, —(CH₂)_{c}-indolyl, —(CH₂)_{c}-benzothiazolyl, wherein c is an integer selected from 0, 1, 2, 3, 4, 5, 6, 7, 8, 9 or 10.

27. The compound according to claim 26, wherein W is selected from —(CH₂)-naphthyl, —(CH₂)-phenyl, —(CH₂)-biphenyl, —(CH₂)-indolyl or —(CH₂)-benzothiazolyl.

28. The compound according to claim 26 or 27, wherein W is —(CH₂)-naphthyl.

29. The compound according to any one of claims 22-28, wherein the linker is characterized by the following Structural Formula (4a):

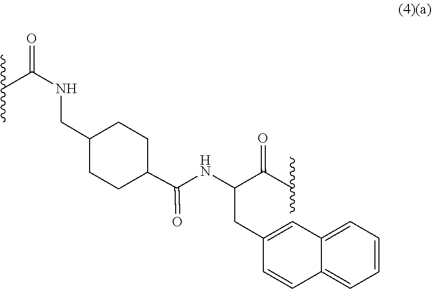 [SMILES: *C(=O)NCC1CCC(C(=O)NC(Cc2ccc3ccccc3c2)C(*)=O)CC1]

30. The compound according to any one of claims 1-29, wherein said compound is characterized by General Formula (1)(b) or (1)(c): 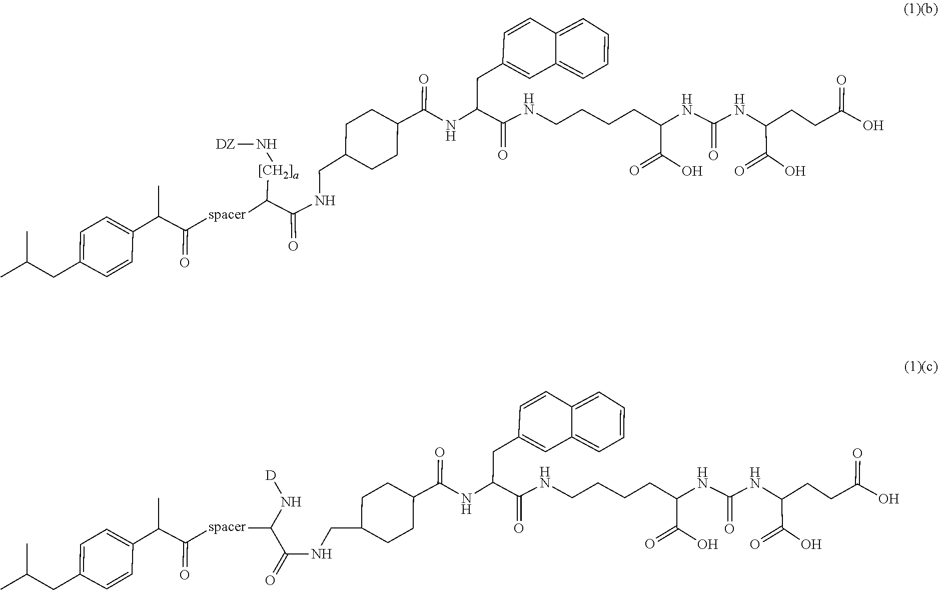
wherein D is a chelator;
spacer is a spacer comprising a C—N bond; and
a is an integer selected from 0, 1, 2, 3, 4, 5, 6, 7, 8, 9, or 10, preferably 0 or 1;
or a pharmaceutically acceptable salt, ester, solvate or radiolabeled complex thereof.

31. The compound according to any one of claims 1-30, wherein the spacer comprises a linear or branched, optionally substituted C₁-C₂₀ hydrocarbyl, more preferably C₁-C₁₂ hydrocarbyl, even more preferably C₂-C₆ hydrocarbyl, even more C₂-C₄ hydrocarbyl, the hydrocarbyl comprising at least one, optionally up to 4 heteroatoms preferably selected from N.

32. The compound according to claim 30 or 31, wherein the spacer comprises —[CHR⁶]ᵤ—NR⁷—, wherein R⁶ and R⁷ are each be independently selected from H and branched, unbranched or cyclic C₁-C₁₂ hydrocarbyl, and u is an integer selected from 1, 2, 3, 4, 5, 6, 7, 8, 9 or 10, wherein u is preferably 2, 3, or 4, more preferably 2 or 4.

33. The compound according to any one of claims 1-32, wherein the spacer is —[CH₂]₂—NH— or —[CH₂]₄—NH—.

34. The compound according to any of claims 1 to 33, wherein the spacer comprises at least one amino acid residue or an amino acid residue side chain, wherein the amino acid is preferably selected from lysine, aspartate, asparagine, diaminobutyric acid, phenylalanine, tyrosine, threonine, serine, proline, leucine, isoleucine, valine, arginine, histidine, glutamate, glutamine, and alanine.

35. The compound according to claim 33 or 34, wherein the spacer comprises or consists of a lysine residue or a lysine residue side chain.

36. The compound according to claim 35, wherein the spacer further comprises a further amino acid residue or a side chain thereof.

37. The compound according to claim 36, wherein the further amino acid residue or the side chain thereof is selected from aspartate, asparagine and diaminobutyric acid.

38. The compound according to any one of claims 1-37, wherein the spacer comprises or consists of Formula (2)(a) or Formula (2)(a)′ or Formula (2)(a)″: 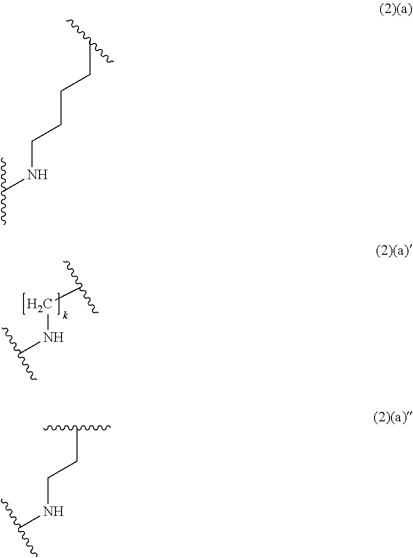
wherein k is an integer selected from 0, 1, 2, 3, 4, 5, 6, 7 and 8, preferably 2, 3 or 4.

39. The compound according to any one of claims 1-38, wherein the spacer comprises or consists of Formula (2)(b): 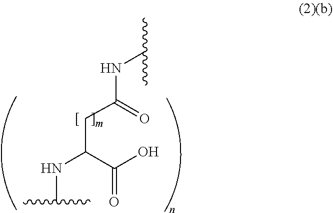
wherein m is an integer selected from 1 or 2, and n is an integer selected from 1, 2, 3, 4 or 5, preferably from 1, 2 or 3.

40. The compound according to any one of claims 1-39, wherein the spacer comprises or consists of Formula (2)(c) or (2)(c)′: 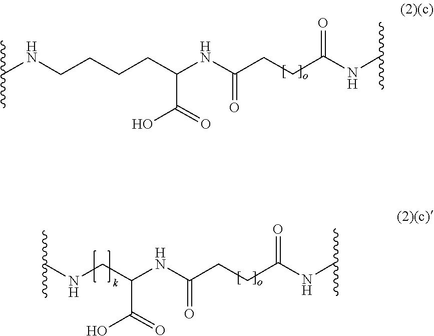
wherein o is an integer selected from 0, 1, 2, 3, 4, 5, 6, 7, 8, 9 or 10 and k is as defined above.

41. The compound according to any one of claims 1-38, wherein the spacer comprises or consists of Formula (2)(d) or (2)(d)′: 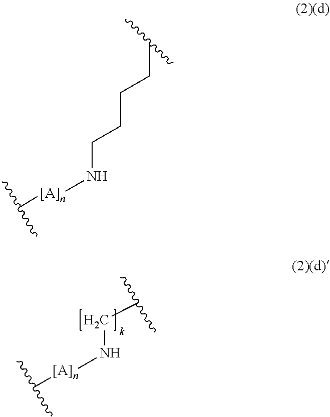
wherein A is an amino acid residue or -[A]ₙ is absent and n is an integer selected from 0, 1, 2, 3, 4, or 5, preferably from 0 or 1, and k is as defined above.

42. The compound according to claim 41, wherein the spacer comprises or consists of Formula (2)(d)(i) or (2)(d)(i)′: 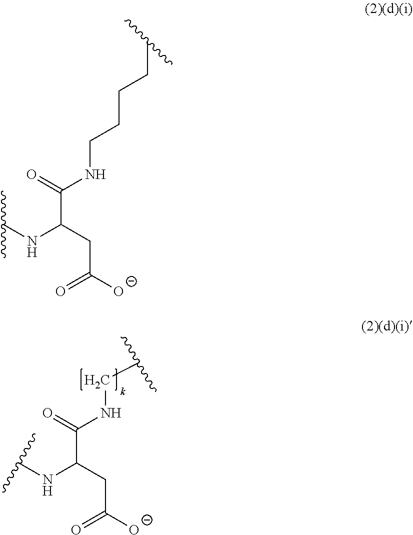
wherein k is as defined above.

43. The compound according to claim 41, wherein the spacer comprises or consists of Formula (2)(d)(ii) or (2)(d)(ii)′: 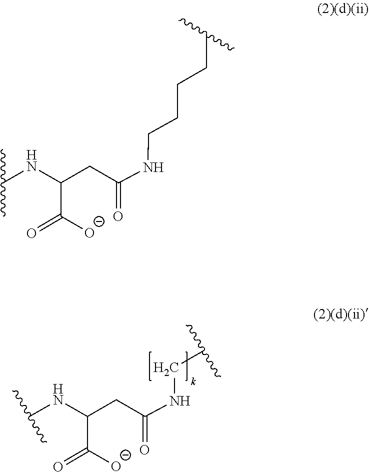
wherein k is as defined above.

44. The compound according to claim 41, wherein the spacer comprises or consists of Formula (2)(d)(iii) or (2)(d)(iii)′: 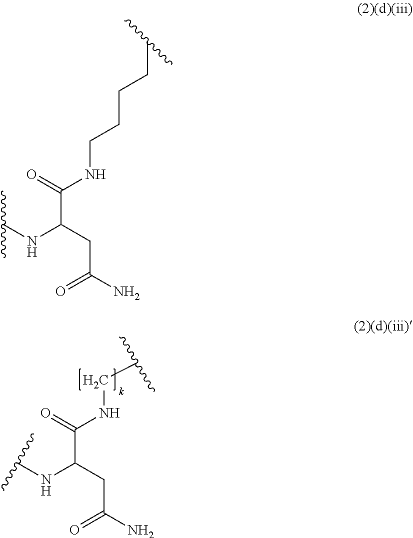
wherein k is as defined above.

45. The compound according to claim 41, wherein the spacer comprises or consists of Formula (2)(d)(iv) or (2)(d)(iv)′: 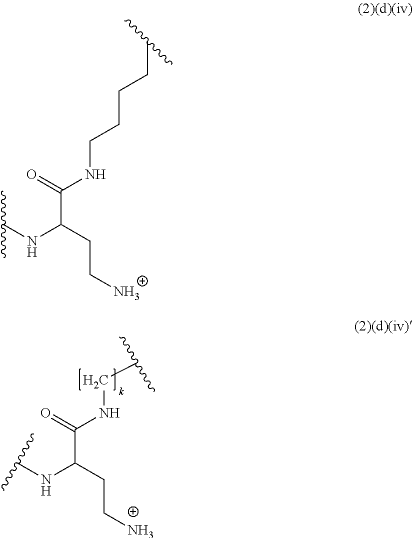
wherein k is as defined above.

46. The compound according to any one of claims 1-45, wherein said compound is characterized by General Formula (1)(n) or (1)(o): 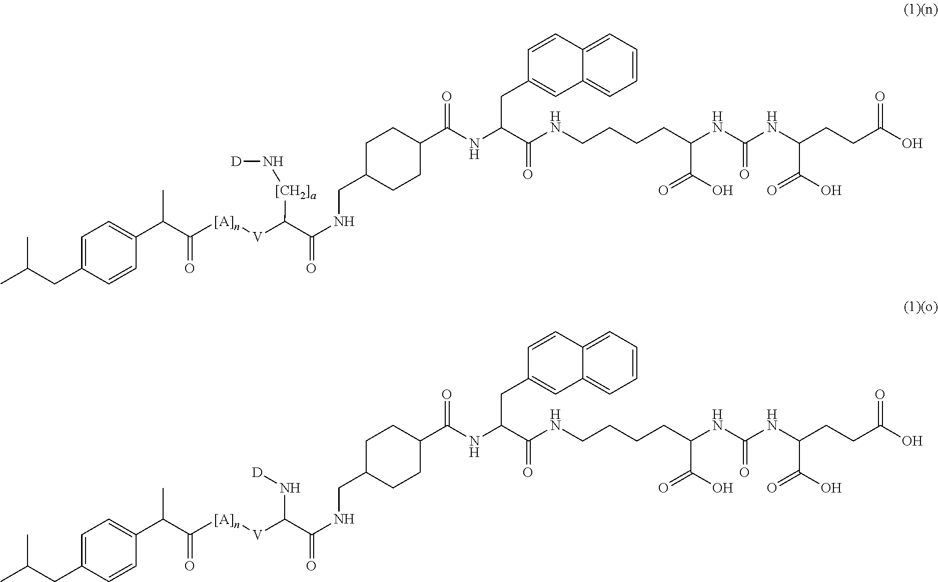
wherein D is a chelator;
A is an amino acid residue, a side chain thereof or —[CHR⁶]ᵤNR⁷—, wherein R⁶ and R⁷ are each be independently selected from H and branched, unbranched or cyclic C₁-C₁₂ hydrocarbyl, and u is an integer selected from 1, 2, 3, 4, 5, 6, 7, 8, 9 or 10, wherein u is preferably 2, 3, or 4, more preferably 2 or 4;
V is absent or selected from a single bond, N or NH, or an optionally substituted C₁-C₁₂ hydrocarbyl comprising up to 3 heteroatoms, wherein said heteroatom is preferably selected from N, wherein V more preferably contains 1 or 2 C—N bond(s);
a is an integer selected from 0, 1, 2, 3, 4, 5, 6, 7, 8, 9, or 10; and
n is an integer selected from 0, 1, 2, 3, 4, or 5, preferably from 0 or 1;
or a pharmaceutically acceptable salt, ester, solvate or radiolabeled complex thereof.

47. The compound according to any one of claims 1-46, wherein said compound is characterized by Formula (7)(a) or (7)(a)′: 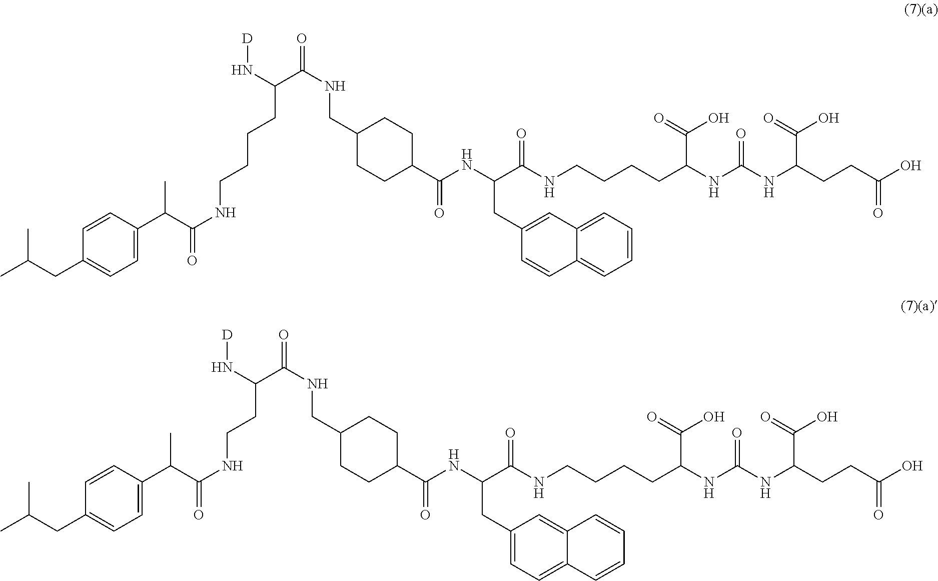
wherein D is a chelator;
or a pharmaceutically acceptable salt, ester, solvate or radiolabeled complex thereof.

48. The compound according to any one of claims 1-46, wherein said compound is characterized by Formula (7)(b) or (7)(b)′: 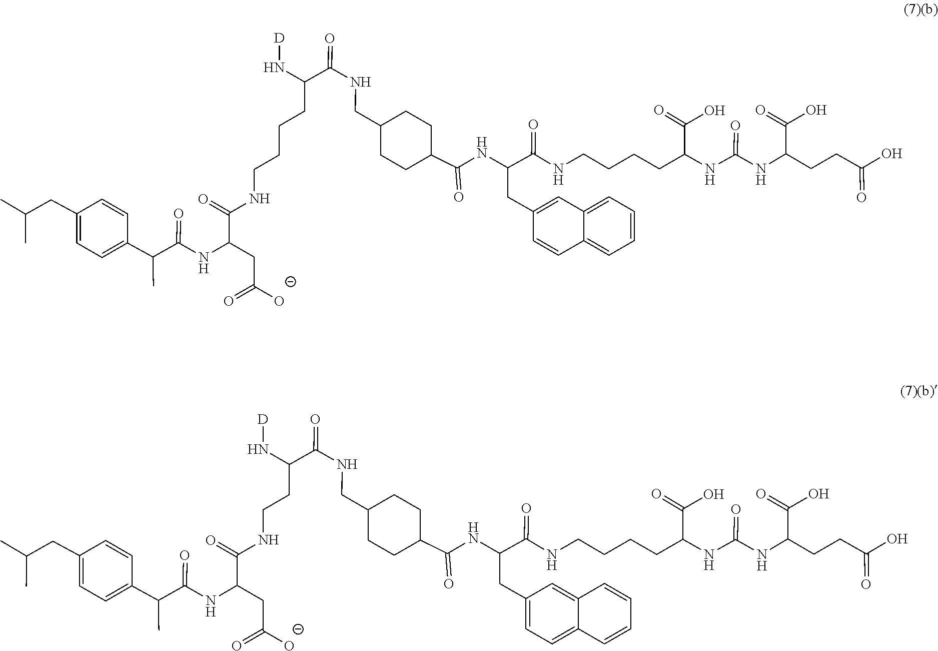
wherein D is a chelator;
or a pharmaceutically acceptable salt, ester, solvate or radiolabeled complex thereof.

49. The compound according to any one of claims 1-46, wherein said compound is characterized by Formula (7)(c) or (7)(c)′: 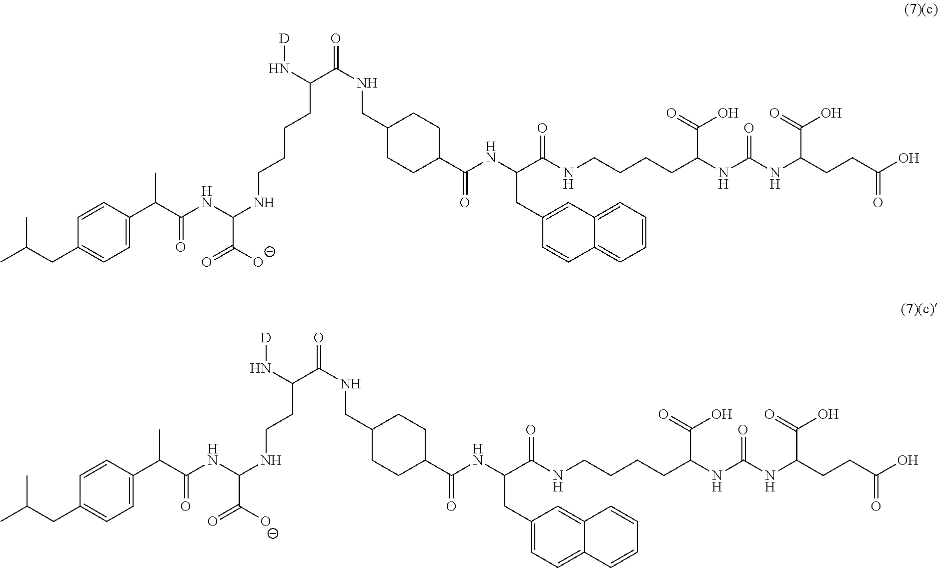
wherein D is a chelator;
or a pharmaceutically acceptable salt, ester, solvate or radiolabeled complex thereof.

50. The compound according to any one of claims 1-46, wherein said compound is characterized by Formula (7)(d) or (7)(d)′: 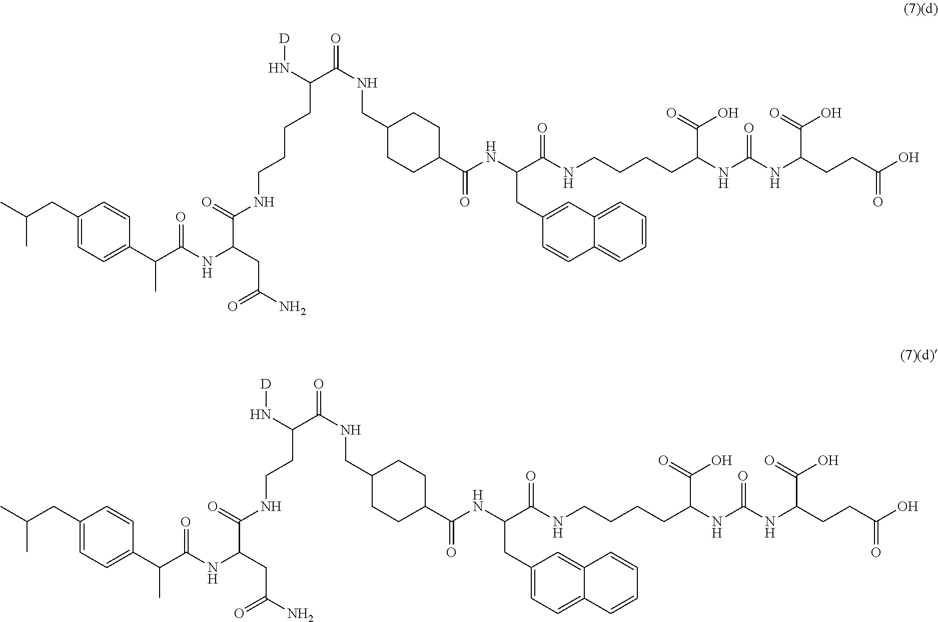
wherein D is a chelator;
or a pharmaceutically acceptable salt, ester, solvate or radiolabeled complex thereof.

51. The compound according to any one of claims 1-46, wherein said compound is characterized by Formula (7)(e) or (7)(e)′: 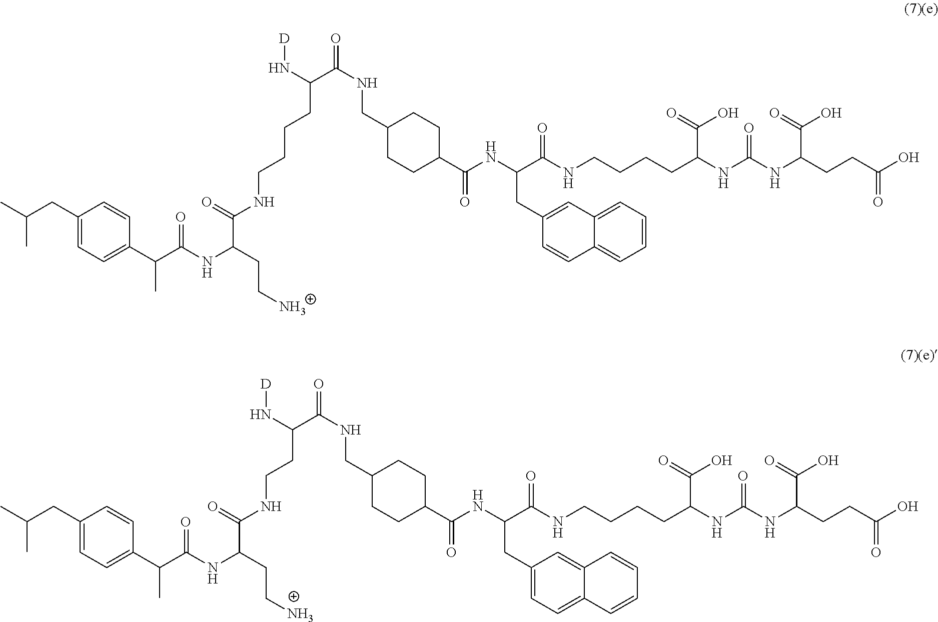
wherein D is a chelator;
or a pharmaceutically acceptable salt, ester, solvate or radiolabeled complex thereof.

52. The compound according to any one of claims 5-51, wherein the chelator (D) is selected from 1,4,7,10-tetraazacyclododecane-1,4,7,10-tetraacetic acid (DOTA), N,N″-bis[2-hydroxy-5-(carboxyethyl)-benzyl]ethylenediamine-N,N″-diacetic acid (HBED-CC), 1,4,7-triazacyclononane-1,4,7-triacetic acid (NOTA), 2-(4,7-bis(carboxymethyl)-1,4,7-triazonan-1-yl)pentanedioic acid (NODAGA), 2-(4,7,10-tris(carboxymethyl)-1,4,7,10-tetraazacyclododecan-1-yl)-pentanedioic acid (DOTAGA), 1,4,7-triazacyclononane phosphinic acid (TRAP), 1,4,7-triazacydononane-1-[methyl(2-carboxyethyl)-phosphinic acid]-4,7-bis[methyl(2-hydroxymethyl)phosphinic acid] (NOPO), 3,6,9, 15-tetraazabicyclo[9,3,1]pentadeca-1(15),11,13-triene-3,6,9-triacetic acid (PCTA), N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide (DFO), and Diethylenetriaminepentaacetic acid (DTPA), or derivatives thereof.

53. The compound according to any one of claims 5-52, wherein the chelator is selected from DOTA, DOTAGA, NODAGA, DO3AP, DO3AP^{PrA} or DO3AP^{ABn}.

54. The compound according to claim 52 or 53, wherein the chelator is DOTA.

55. The compound according to any one of claims 1-54, wherein said compound is characterized by Structural Formula (8)(a) or (8)(a)′: 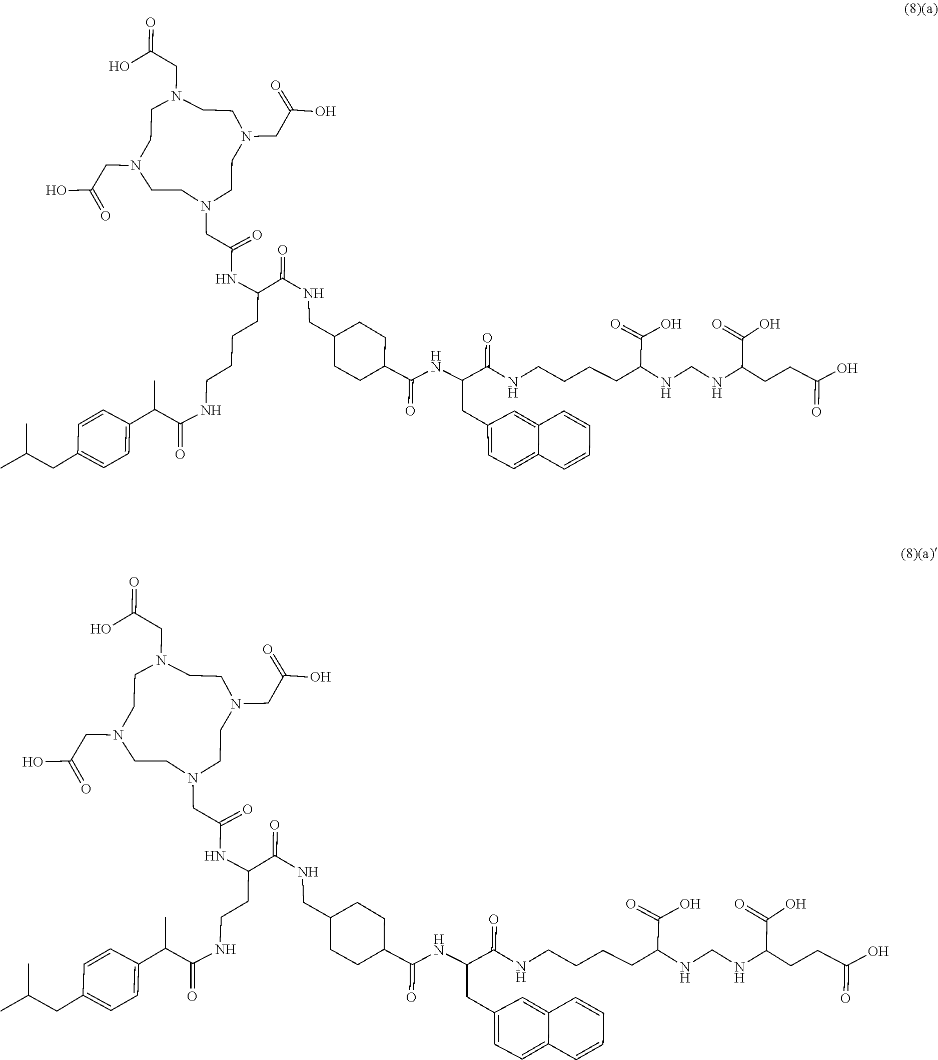 [SMILES: CC(C)Cc1ccc(C(C)C(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NCNC(CCC(=O)O)C(=O)O)C(=O)O)CC2)cc1 ; CC(C)Cc1ccc(C(C)C(=O)NCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NCNC(CCC(=O)O)C(=O)O)C(=O)O)CC2)cc1]
or a pharmaceutically acceptable salt, ester, solvate or radiolabeled complex thereof.

56. The compound according to any one of claims 1-54, wherein said compound is characterized by Structural Formula (8)(b) or (8)(b)′: 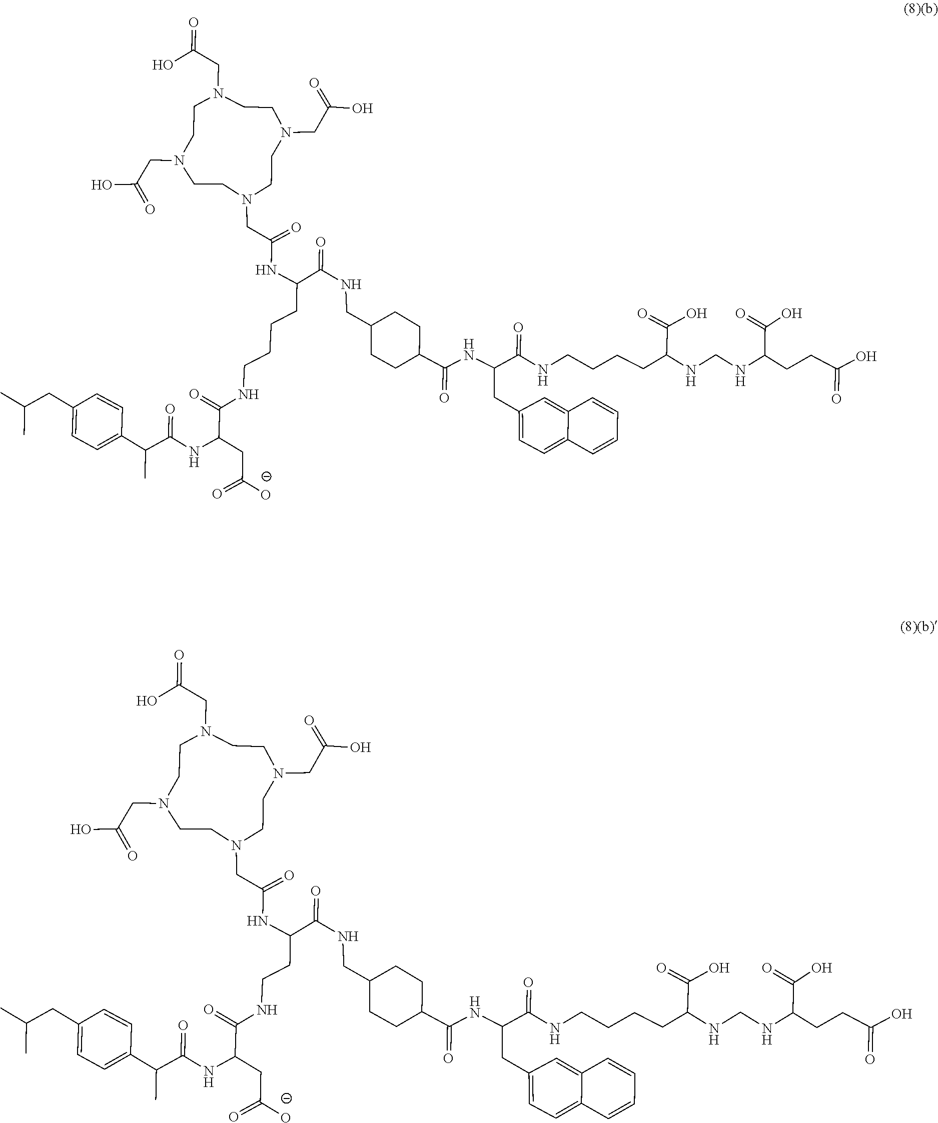 [SMILES: CC(C)Cc1ccc(C(C)C(=O)NC(CC(=O)[O-])C(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NCNC(CCC(=O)O)C(=O)O)C(=O)O)CC2)cc1 ; CC(C)Cc1ccc(C(C)C(=O)NC(CC(=O)[O-])C(=O)NCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NCNC(CCC(=O)O)C(=O)O)C(=O)O)CC2)cc1]
or a pharmaceutically acceptable salt, ester, solvate or radiolabeled complex thereof.

57. The compound according to any one of claims 1-54, wherein said compound is characterized by Structural Formula (8)(c) or (8)(c)′: 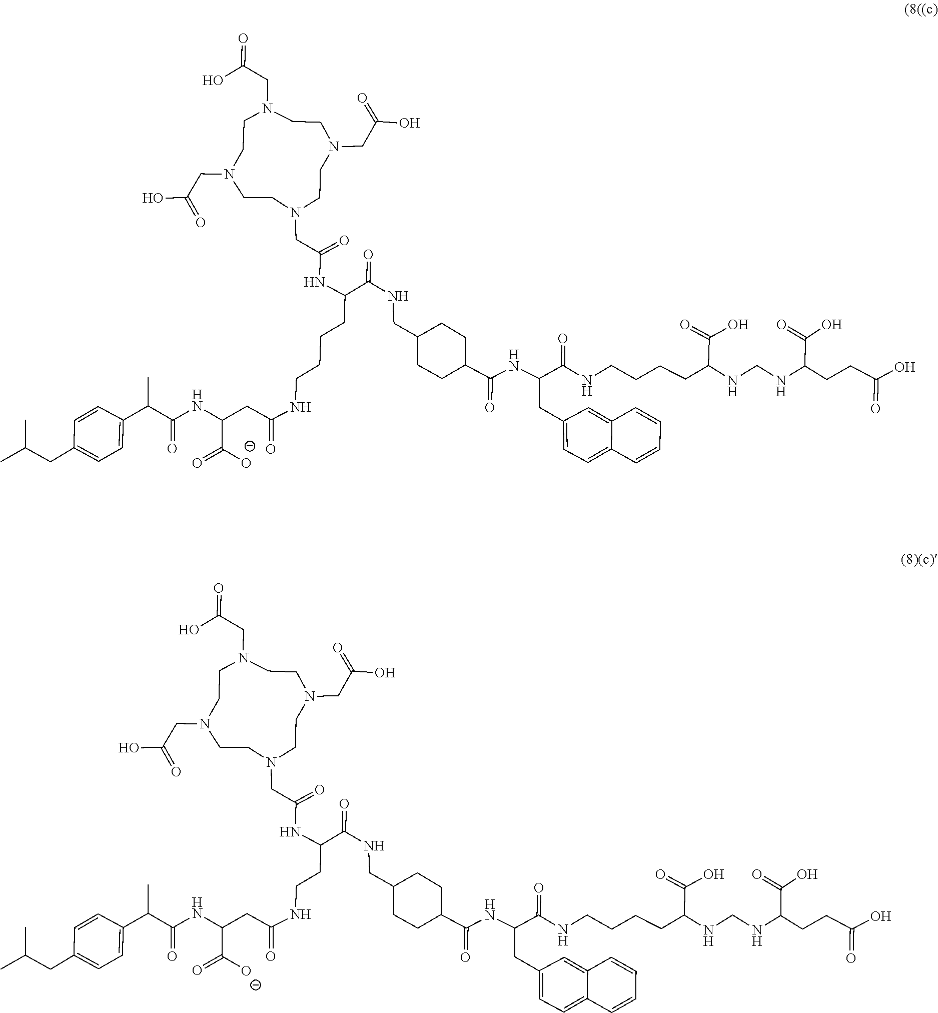 [SMILES: CC(C)Cc1ccc(C(C)C(=O)NC(CC(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NCNC(CCC(=O)O)C(=O)O)C(=O)O)CC2)C(=O)[O-])cc1 ; CC(C)Cc1ccc(C(C)C(=O)NC(CC(=O)NCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NCNC(CCC(=O)O)C(=O)O)C(=O)O)CC2)C(=O)[O-])cc1]
or a pharmaceutically acceptable salt, ester, solvate or radiolabeled complex thereof.

58. The compound according to any one of claims 1-54, wherein said compound is characterized by Structural Formula (8)(d) or (8)(d)′: 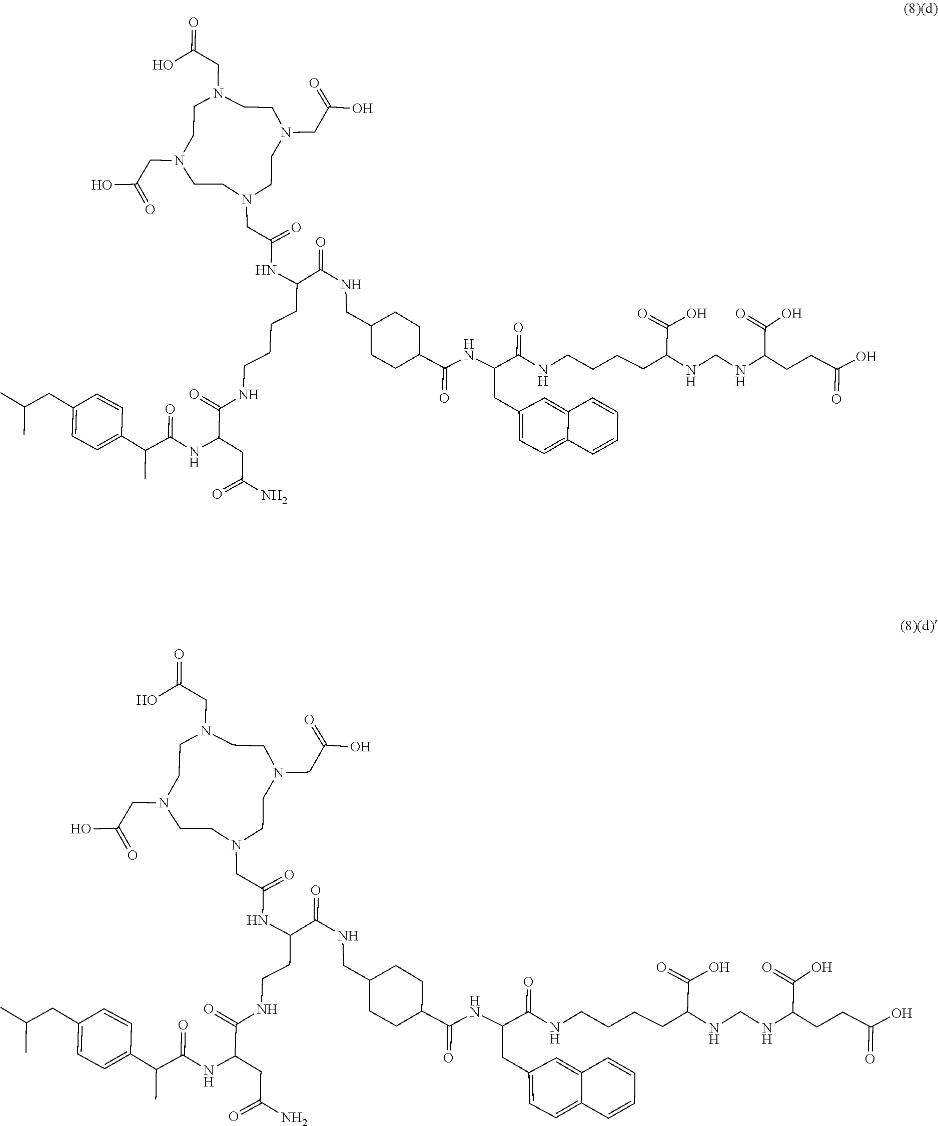 [SMILES: CC(C)Cc1ccc(C(C)C(=O)NC(CC(N)=O)C(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NCNC(CCC(=O)O)C(=O)O)C(=O)O)CC2)cc1 ; CC(C)Cc1ccc(C(C)C(=O)NC(CC(N)=O)C(=O)NCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NCNC(CCC(=O)O)C(=O)O)C(=O)O)CC2)cc1]
or a pharmaceutically acceptable salt, ester, solvate or radiolabeled complex thereof.

59. The compound according to any one of claims 1-54, wherein said compound is characterized by Structural Formula (8)(e) or (8)(e)′: 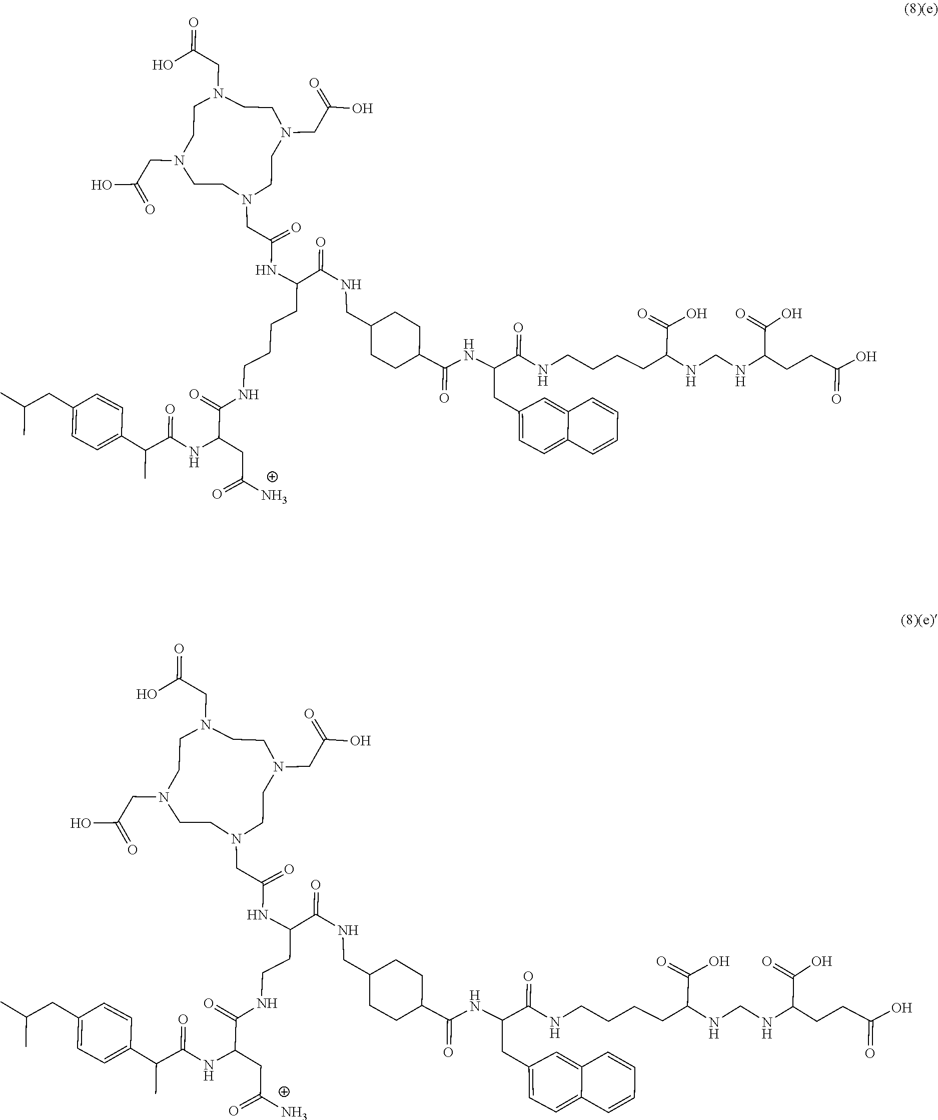 [SMILES: CC(C)Cc1ccc(C(C)C(=O)NC(CC([NH3+])=O)C(=O)NCCCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NCNC(CCC(=O)O)C(=O)O)C(=O)O)CC2)cc1 ; CC(C)Cc1ccc(C(C)C(=O)NC(CC([NH3+])=O)C(=O)NCCC(NC(=O)CN2CCN(CC(=O)O)CCN(CC(=O)O)CCN(CC(=O)O)CC2)C(=O)NCC2CCC(C(=O)NC(Cc3ccc4ccccc4c3)C(=O)NCCCCC(NCNC(CCC(=O)O)C(=O)O)C(=O)O)CC2)cc1]
or a pharmaceutically acceptable salt, ester, solvate or radiolabeled complex thereof.

60. Use of a compound according to any one of claims 1 to 59 for the preparation of a radiolabeled complex.

61. A compound according to any of claims 1 to 59 for use as a medicament or as a precursor of a medicament.

62. A radiolabeled complex comprising a radionuclide and a compound according to any one of the preceding claims.

63. The radiolabeled complex according to claim 62, wherein the radiolabel is selected from the group consisting of ⁹⁴Tc, ^{99m}Tc, ⁹⁰In, ¹¹¹In, ⁶⁷Ga, ⁶⁸Ga, ⁸⁶Y, ⁹⁰Y, ¹⁷⁷Lu, ¹⁵¹Tb, ¹⁸⁶Re, ¹⁸⁸Re, ⁶⁴Cu, ⁶⁷Cu, ⁵⁵Co, ⁵⁷Co, ⁴³Sc, ⁴⁴Sc, ⁴⁷Sc, ²²⁵Ac, ²¹³Bi, ²¹²Bi, ²¹²Pb, ²²⁷Th, ¹⁵³Sm, ¹⁶⁶Ho, ¹⁵²Gd, ¹⁵³Gd, ¹⁵⁷Gd, or ¹⁶⁶Dy.

64. The radiolabeled complex according to claim 62 or 63, wherein the radiolabel is ¹⁷⁷Lu.

65. A pharmaceutical composition comprising the compound according to any one of claims 1 to 60, or a radiolabeled complex according to any one of claims 62-64, and, optionally, a pharmaceutically acceptable carrier, diluent and/or excipient.

66. A kit comprising a compound according to any one of claims 1 to 60 or a pharmaceutically acceptable salt, ester, solvate or radiolabeled complex thereof, a radiolabeled complex according to any one of claims 62-64 or a pharmaceutical composition according to claim 64.

67. The compound according to any one of claims 1 to 60, the radiolabeled complex according to any one of claims 62-64, the pharmaceutical composition according to claim 65 or the kit according to claim 66 for use in medicine and/or diagnostics.

68. The compound according to any one of claims 2 to 60, the radiolabeled complex according to any one of claims 62-64, the pharmaceutical composition according to claim 65 or the kit according to claim 66 for use in a method of detecting the presence of (isolated) cells and/or tissues expressing prostate-specific membrane antigen (PSMA).

69. The compound according to any one of claims 2 to 60, the radiolabeled complex according to any one of claims 62-64, the pharmaceutical composition according to claim 65 or the kit according to claim 66 for use in a method of diagnosing, treating and/or preventing cancer, preferably prostate cancer, pancreatic cancer, renal cancer or bladder cancer.

70. The compound, radiolabeled complex, pharmaceutical composition or kit for use according to any one of claims 67-69, wherein said method or use comprises
(a) administering said compound, radiolabeled complex or pharmaceutical composition to a patient, and
(b) obtaining a radiographic image from said patient.

71. An in vitro method of detecting the presence of cells and/or tissues expressing prostate-specific membrane antigen (PSMA) comprising
(a) contacting said PSMA-expressing cells and/or tissues with a compound, radiolabeled complex, pharmaceutical composition or kit according to any one of the preceding claims;
(b) applying detection means, optionally radiographic imaging, to detect of said cells and/or tissues.

72. The compound, the radiolabeled complex, the pharmaceutical composition or the kit for use according to any one of claims 67-70, or the method according to claim 71, wherein radiographic imaging comprises positron emission tomography (PET) or single-photon emission computed tomography (SPECT).

73. The compound, the radiolabeled complex, the pharmaceutical composition or the kit for use according to any one of claims 67-70 or 72, or the method according to claim 71 or 72, wherein said one or more cells or tissues comprise (optionally cancerous) prostate cells or tissues, (optionally cancerous) spleen cells or tissues, or (optionally cancerous) kidney cells or tissues.

74. The compound, the radiolabeled complex, the pharmaceutical composition or the kit for use according to any one of claims 67-70 or 72-73, or the method according to any one of claims 71-73, wherein the presence of PSMA-expressing cells or tissues is indicative of a prostate tumor (cell), a metastasized prostate tumor (cell), a renal tumor (cell), a pancreatic tumor (cell), a bladder tumor (cell), and combinations thereof.
